SEQUENCE LISTING (288 sequences)

SEQ ID NO: 1 - protein, 118 aa, Homo sapiens
QVQLVQSGAEMKKPGASVKISCKASGYTFTSDYMHWVRQAPGQGLEWMGIVNPSGSGTRYAQKFQGRVTMTRDTPTKTFYIELTRLKSDDTAVYYCASGILTGLFDYWGQGTLVTVSS

SEQ ID NO: 2 - protein, 118 aa, Homo sapiens
QVQLVQSGAEVKKPGASVKVSCQASGNTFTNYYVHWVRQAPGQGLEWMGIISPSGDGTRYAQKFQGRVTMTRDTSTTTVYMELSSLRSEDTAVYYCARGSNWGPWDYWGQGTLVTVSS

SEQ ID NO: 3 - protein, 117 aa, Homo sapiens
QVQLVESGGGLVRPGRSLRLSCTGSGFNFGDYAMSWFRQVPGTGLEWVGFIRNKAYGGTPEHAASLRGRFTISRDDSRDIAYLQMNSLTREDTGVYFCATTHAGVSWGQGTLVTVSS

SEQ ID NO: 4 - protein, 118 aa, Homo sapiens
QVQLVQSGAEVKQPGASVKISCKSSEYAFTTYYVHWVRQAPGQGPEWMGLINPSGGGTSYEQKFRGRVTMTRDTSTGTVYMDLTSLRSEDTAVYYCASGFRGPLFDYWGQGTLVTVSS

SEQ ID NO: 5 - protein, 118 aa, Homo sapiens
QVQLVQSETEVKKPGASVKVSCKASGNTFTSYYFHWVRQAPGQGLEWMGIINPSGDGTSYAPKFQGRITMTRDTSTNTVYMELSSLRSEDTAVYYCASGFRGPLFDYWGQGTLVTISS

SEQ ID NO: 6 - protein, 117 aa, Homo sapiens
QVQLVESGGGLVKPGRSLRLSCTGSGFNFGDYAMSWFRQVPGTGLQWVGFIRNKAYGGTPEHAASLRGRFTISRDDSRDIAYLQMNSLTSEDTGIYFCATTHAGVSWGQGTLVTVSS

SEQ ID NO: 7 - protein, 115 aa, Homo sapiens
QVQLVQSGAEVKKPGASVKVSCKASGYIFSNYYMHWVRQAPGQGLEWMGLINPTGVNTSYAQKFQGRLTMTRDTSTSTLSMELSNLTSEDTAMYYCSRGSSPWDWGQGTLVTISS

SEQ ID NO: 8 - protein, 114 aa, Homo sapiens
QVQLVESGGGLVQPGGSLRLSCAASGFPFSNYWMTWVRQAPGKGLEWVANIKRDGSEKYYVDSVKGRFTISRDNAKNSLDLQMISLRADDTAVYYCATWFGIHWGKGTLVTVSS

SEQ ID NO: 9 - protein, 120 aa, Homo sapiens
EVQLLESGGGMVQPGGSLRLSCAASGFTFSNYAMIWVRQAPGKGLEWVSVISGGGGGTYYADSVKGRFTISRDNSKNTVFLQMNSLRAEDSAVYYCAKFLQPQHLVFDYWGRGTLVTVSS

SEQ ID NO: 10 - protein, 118 aa, Homo sapiens
QVQLEQSGAEVKKPGASVKVSCKASGNDFATYYVHWVRQAPGQGLEWMGIINPSGDGTSYAQKFQGRVTMTRDTSTNRVFMELRSLRSEDTAVYYCASGFTGPLFDYWGQGTLVTISS

SEQ ID NO: 11 - protein, 117 aa, Homo sapiens
EVQLVQSGAEVKKPGASVKVSCKVSGNTFTNYYIHWVRQAPGQGLEWQGTIDPSCGGTRYAQKLQGRVTMTRDTSTKTVYMYVSTLRSEDTAVYYCAMTLYGVFDYWGQGTLVTVSS

SEQ ID NO: 12 - protein, 117 aa, Homo sapiens
QVQLVQSGAEVKKPGASVKVSCKASGNIFTSYYMHWVRQAPGQGLEWLGIINPSGDGTSYAQKFQGRVTMTKDTSTNTVYMYVSSLRSEDTAVYYCAMTLYGVFDYWGQGTLVTVSS

SEQ ID NO: 13 - protein, 120 aa, Homo sapiens
EVQLVESGGGLVHPGGSLRLPCAVSGFTFSNYAMSWVRQAPGKGLQWVSVISGGSGARYYADSVKGRFTISRDNSKNTLYLQMQSLRAEDTAVYYCAKWTHYGDFGVDLWGRGTLVTVSS

SEQ ID NO: 14 - protein, 120 aa, Homo sapiens
QVQLVQSGAEVKKPGASVKVSCKASGYTFTSYYMHWVRQAPGQGLEWMGIITPSGDNTRYAQKFQGRVTMTRDTSTSTLFMELSSLRSDDTAVYYCASGDSSDSSSYHYWGQGTLVTVSS

SEQ ID NO: 15 - protein, 117 aa, Homo sapiens
QVQLVQSGAEVKKPGASVKVSCKASGYNFIDHYMQWVRQAPGQGLQWMGIISPSAGYTAYAQRFQGRVTLTGDTSTSTVYMELRSLRSEDTAIYYCARLRFGVNDHWGQGTLVTVSS

SEQ ID NO: 16 - protein, 123 aa, Homo sapiens
QVQLQESGPGLVKPSQTLSLTCTVSGGSISSGGYYWTWIRQHPGKGLEWIGYISYRGNTFYNPSLKSRAAMSIDTSKNQFSLNLSSVTAADTAVYHCAGTSAGGMGSHAMDVWGQGTTVTVSS

SEQ ID NO: 17 - protein, 117 aa, Homo sapiens
EVQLVQSGAEVKKPGASLKVSCKASGYTFTDYYMHWVRQAPGQGLEWMGIIKPSAGNTRNAQKFQGRVTMTRDTSTSTVYMELSALRFEDTAVYYCARGGVHGLDYWGQGTLVTVSS

SEQ ID NO: 18 - protein, 117 aa, Homo sapiens
QVQLVQSGAEVRKPGASVKVSCKASGDTFASNYMLWVRQAPGQGLEWMGIINPSGDRTSYAQKFQGRVTMTRDTSTSTVYMELRSLRSEDTAVYFCARLRFGVNDYWGQGTLVTVSS

SEQ ID NO: 19 - protein, 119 aa, Homo sapiens
QVQLLESGGGLAQPGGSLRLSCAASGFTFSRYAMSWVRQAPGKGLEWVSVTSGGGGSSYYADSVKGRFTISRDNSKNTLYLQMISLRAEDTAVYYCAKVGTTMVYFDYWGQGTLVTVSS

SEQ ID NO: 20 - protein, 118 aa, Homo sapiens
EVQLLESGGGLVQPGESLRLSCAVSGFTFSSYAMAWVRQAPGKGPEWVSVISGASGSSYYTDSVKGRFTISRDNSKNTLYLQMNSLRAEDTAVYYCAKVKQVLYFDYWGQGTLVTVSS

SEQ ID NO: 21 - protein, 117 aa, Homo sapiens
EVQLVQSGAEVKKPGASVKVSCQASGFTFSNYYMNWVRQAPGRGLEWMGIINPSGQSTSYPQKFQGRVTMTRDTSTSTVNMELSSLKSEDTAVYYCVRVARGGFDIWGQGTVVTVSS

SEQ ID NO: 22 - protein, 119 aa, Homo sapiens
EVQLVESGGGLVQPGGSLRLSCAVSGLTFSRHAMSWVRQTPGKGLGWVSVISGGGGGTYYADSVKGRFTISRDNSKNTLYLLMNSLRVEDTAIYYCAKVAGGTVFFDYWGQGALVTVSS

SEQ ID NO: 23 - protein, 118 aa, Homo sapiens
QVQLVQSGAEVKTPGASVKVSCKASGDTFTNYYVHWVRQAPGQGLEWMGIINPSGYGTTYAQKFQGRVTMTRDTSTGTVYMELSSLKSEDTAVYYCARGSNWGPWDYWGQGTLVTVSS

SEQ ID NO: 24 - protein, 116 aa, Homo sapiens
QVQLVQSGAEVKKPGASVKVSCKASGTTFTSDYMHWVRQAPGQGLEWMGIIDPSGGGTSYARKFQGRVTMTRDTSTSTVYMELGTLRSEDTAVYFCARGSSGWYFWGQGTLVTVSS

SEQ ID NO: 25 - protein, 119 aa, Homo sapiens
QVQLVQSGAEVTKPGASVTVSCKASGYTFTNYYMHWVRQAPGQGLEWMGIIKPSGGNTIYAQKFQGRVTMTRDTSTSTVYLELSSLRSEDTAVYYCANTMIRGIIITHWGQGTLVTVSS

SEQ ID NO: 26 - protein, 120 aa, Homo sapiens
EVQLVQSGAEVKKPGASVKLSCKASGYTFTTYYMHWVRQAPGQGLEWIGIINPSGAGTSYAQQFQGRITMTRDTSTSTLYMELSSLRSEDTAVYYCASPPRGSSSALGVWGQGTLVTVSS

SEQ ID NO: 27 - protein, 118 aa, Homo sapiens
QVQLLQSGPEVKRPGASVKVSCKGSADTLSGYYMHWVRQAPGQGLEWMGLITPNGAGTRYPQNFQGRVTMTRDTSTSAIYVELRSLTFEDTAVYYCARGEDNGSFFLWGRGTLVTVSS

SEQ ID NO: 28 - protein, 118 aa, Homo sapiens
QVQLVQSGDEVKRPGASVKVSCKASGSTLSGYYMHWVRQAPGQGLEWVGLINPSGTGTAYAQKLQGRVTMTRDTSTSTVYMHLSRLRSEDTALYYCARMEDNGSYFLWGQGTLVTVSS

SEQ ID NO: 29 - protein, 118 aa, Homo sapiens
QVQLLDSGGTLVQPGGSLRLSCVASGFTFSNYAMSWVRQTPGKGLEWVSVISGRGGATYYADSVKGRFSISRDNSKNTLFLQMNSLRAEDTAVYYCAKLLMVMVFDHWGQGTVVTVSS

SEQ ID NO: 30 - protein, 133 aa, Homo sapiens
QVQLHESGPGQVKPSQTLSLTCTVSGASINSGRYYWSWLRQRPGKGLEWIGYIHYTGSTYYNPSLRSRVVFSIDTSSSQFSLKLTAVTAADTAMYYCASLWRESCTTAGCYPKDTSLYYFDYWGQGILVTVSS

SEQ ID NO: 31 - protein, 117 aa, Homo sapiens
EVQLVQSGTEVRQPGASVRVSCKASGYTFTDSYIHWVRQAPGQGLEWMGIIKPSGGNTRYAQRFQGRVTMTRDTSTSTVYMELSSLRSEDTAVYYCARDSRGPGIFWGQGTLVTVSS

SEQ ID NO: 32 - protein, 117 aa, Homo sapiens
EVQLVQSGAEVKKPGASVKISCKASGYTFSSSYMYWVRQAPGQGLEWMGIIKPSGGNTRYAQKFQGRVTMTWDTSTSTVYMELSSLTSDDTAVYYCAGDLRGVGGSWGQGTLVTVSS

SEQ ID NO: 33 - protein, 117 aa, Homo sapiens
EVQLVQSGAEVKKPGASVKVSCEASGYTLTDYYLHWVRQAPGQGLEWMGIIKPSGGNTIYAQTLRGRVTMTRDTSASKVYMELRSLRHDDTAVYFCARGGRHAHDIWGQGTMVTVSS

SEQ ID NO: 34 - protein, 115 aa, Homo sapiens
EVQLVESGGGLVKPGGSLRLSCAASGLPFSGAWMTWIRQAPGKAPEWVGRIKSKSDGGTIDYAAPVKGRFTISRDDSRNTVFLHMDSLKVEDTAVYYCNWNLDYWGQGTLVTVSS

SEQ ID NO: 35 - protein, 117 aa, Homo sapiens
QVQLVQSGAEVKKPGASVKVSCKASGFTFSDYYMNWVRQAPGQGPEWMGIINPSGTGTVYAQKFQGRVTMTRDTSTSTVYMELSSLTSEDTAVYYCARVVRGASSLWGQGTLVTVSS

SEQ ID NO: 36 - protein, 130 aa, Homo sapiens
QVQLQESGPGLVKPSQTLSLTCTVSGSGGSISSGGYYWNWIRQHPGKGLEWIGYIHYSGSTYYHPSLKSRITISVDTSKNQFSLKLSSVTAADTAVYYCARGFRENYDNSGYSSYYFDYWGQGTLVTVSS

SEQ ID NO: 37 - protein, 119 aa, Homo sapiens
EVQLVQSGAEVRKPGASVKLSCKTSGYTFTNFYIHWVRQAPGQGLEWMGIISTSAGSTRYTQKFQGRVTMTRDTSTSTVYMELSSLRYDDTALYFCARDLYGSRNFHYWGQGTLVTVSS

SEQ ID NO: 38 - protein, 116 aa, Homo sapiens
QVQLVQSGAEVKKPGASVKVSCKASGTTFTSDYMHWVRQAPGQGLEWMGIIDPSGGGTSYAQKFQGRVTMTRDTSTSTVYLELSTLRSEDTAVYFCARGSSGWYFWGQGTLVSVSS

SEQ ID NO: 39 - protein, 118 aa, Homo sapiens
EVQLVQSGAEMKKPGASVKISCKASGYTFTSDYMHWVRQAPGQGLEWMGIINPSGSGTRYAQKFQGRVTMTRDTPTTTFYMELTRLRSEDTAVYYCASGILTGLFDYWGQGTLVTVSS

SEQ ID NO: 40 - protein, 118 aa, Homo sapiens
EVQLVQSGAEMKKPGASVKISCKASEYTFTNDYMHWVRQAPGQGLEWMGIINPSGSGTRYAQKFQGRVTMTRDTPTNTFYIELTRLRSEDTAVYYCASGILTGLFDYWGQGTLVTVSS

SEQ ID NO: 41 - protein, 110 aa, Homo sapiens
DVVMTQSPGTLSLSAGERATLSCRASQTMTKNYVAWYQQKPGQAPRLLIYGASTRATGIPDRFSGSGSGTDFTLTISRLAPEDFAVYYCLQYGSSPPIFTFGPGTKVEIK

SEQ ID NO: 42 - protein, 110 aa, Homo sapiens
EIVLTQSPGTLSLSPGERATLSCRASQSVRRNYLAWYQQKPGQAPRLLIFGASSRATGIPDRFSGSGSGTDFTLTISRLEPEDFAVYYCQQYDSSPPIFTFGPGTKVDIK

SEQ ID NO: 43 - protein, 113 aa, Homo sapiens
EIVLTQSPDSLAVSLGETASINCKSSLTVLNPSNNKFHLSWYQQKPGQPPKLLIYWASTRESGVPDRFSGSGSGTDFTLTITSLQAEDVAVYYCQQYYSLPITFGQGTRLEIK

SEQ ID NO: 44 - protein, 110 aa, Homo sapiens
DVVMTQSPGTLSLSPGERATLSCRASQSVTSNYLAWYQQKPGQAPRLLIYGASTRATGIPDRFTGSGSGTDFTLTIRRLEPEDFAVYYCQQYSSSPPRLTFGPGTKVEIK

SEQ ID NO: 45 - protein, 110 aa, Homo sapiens
DVVMTQSPGTLSLSPGERATLSCRASQSVRKNYLAWFQQKPGQAPRLLIYGASSRATGIPDRFSGSGSGTDFTLIISRLEPEDFAVYYCQQYDSSPPRLTFGPGTKVEIK

SEQ ID NO: 46 - protein, 113 aa, Homo sapiens; identical to SEQ ID NO: 43

SEQ ID NO: 47 - protein, 110 aa, Homo sapiens
QPVLTQEPSLTVSPGETVTLTCASSTGAVTSGFYANWFQQKPGQPPRSLIYSSYNKHSWTPARFSGSLLGGKAALTLSGVQPEDEAEYYCLLYFGAAQLLFGGGTKLTVL

SEQ ID NO: 48 - protein, 113 aa, Homo sapiens
DIVMTQSPDSLAVSLGERATINCKSSQSLLYSSSNKNYLAWFQQKPGQPPKLLIYYASTRESGVPDRFSGSGSGTDFTLTISSLQAEDVAVYYCQQHYSPPHTFGPGTKVEIK

SEQ ID NO: 49 - protein, 108 aa, Homo sapiens
DIVMTQSPGTLSLSPGERATLSCRASQRVSNSYLAWYQHKPGQGPRLLMYGASTRATGIPDRFSGSGSGTDFTLTISRLEPEDFAVYYCQQYGRSPFTFGPGTKVEIK

SEQ ID NO: 50 - protein, 110 aa, Homo sapiens
DIVMTQSPGTLSLSPGERATLSCRASQRVINRYLAWYQQKPGQAPRLLIYGASSRATGIPDRFTGSGSETDFTLTIRRLEPEDFAVYYCQHYASSPPRLTFGPGTKLEIK

SEQ ID NO: 51 - protein, 110 aa, Homo sapiens
DVVMTQSPGTLSLSPGESATLSCRASQSVRNKYLAWYQQKPGQAPRLLIYGASTRATGIPDRFRGSGSETDFTLTISRLEPDDFAVYYCQQYDSSPPSFTFGPGTKLEIK

SEQ ID NO: 52 - protein, 110 aa, Homo sapiens
EIVMTQSPGTLSLSPGESATLSCRASQSVRNNYLAWYQQKPGQAPRLLIYGASTRATGIPDKFRGSGSGTDFTLTISRLEPDDFAVYYCQQYDSSPPSFTFGPGTKLEIK

SEQ ID NO: 53 - protein, 108 aa, Homo sapiens
EIVLTQSPGTLSLSPGERATLSCRASHGVSSSSVAWYQQKPGQAPRLLIYDASSRATGIPDRISGSGSGTDFTLTISRLEAEDFAVYYCQQYGSPPYTFGQGTKLEIK

SEQ ID NO: 54 - protein, 109 aa, Homo sapiens
DIVMTQSPSSVSASVGDRVTITCRASQSISRWLAWYQQKPGKAPKLLIYAVSTLQSGVPSRFSGSRSGTDFTLTISSLQPEDFATYYCQQANSSPPSFTFGPGTKLEIK

SEQ ID NO: 55 - protein, 110 aa, Homo sapiens
DVVMTQSPGTLSLSPGERATLSCRASQSVRSNYLAWYQQKPGQAPRLLIYGVSSRATGIPDRFSGSGSGTDFTLTISRLEPEDFAVYYCQQYDSSPPIFTFGPGTKLEIK

SEQ ID NO: 56 - protein, 109 aa, Homo sapiens
QSVLTQPPSVSAAPGQKVTISCSGSSSNIGINFVSWYQQLPGTAPKLLIYDNNKRPSGIPDRFSGSKSGTSATLDITGLQTGDDADYYCGAWDSSLGWVFGGGTKLTVL

SEQ ID NO: 57 - protein, 110 aa, Homo sapiens
QSVLTQPPSVSAPPGQKVTISCSGSSSNIGNNYVSWYQQLPGTAPKLLIHENNQRPSGIPDRFSGSKSGTSATLGITGLQTGDEADYYCGTWDTNLGAFVFGAATRVTVL

SEQ ID NO: 58 - protein, 110 aa, Homo sapiens
EIVMTQSPGTLSLSPGDRATLSCRASQSVTSNYLAWYQQKPGQAPRLLIYGASTRATGIPDRFSGSGSGTDFTLTISRLEPEDFAVYYCEQYGSSPPIFTFGPGTKVDIK

SEQ ID NO: 59 - protein, 108 aa, Homo sapiens
EIVLTQSPGTLSLSPGERATLSCRASQSVSSSHLAWYQQKPGQAPRLLIHGASSRATGIPDRFSGSGSGTDFTLTISRLEPEDFAVYHCQQYGDSPPTFGQGTKVDIK

SEQ ID NO: 60 - protein, 107 aa, Homo sapiens
EIVLTQSPGTLSLSPGERATLSCRARQSVSSSLAWYQQRPGQAPRLLIYDASTRATGFPDRFSGSGSGTDFTLTISRLEPEDFAVYYCQQYGSSPPTFGQGTKLEIK

SEQ ID NO: 61 - protein, 109 aa, Homo sapiens
DVVMTQSPTSLSASVGDRVTITCRASQSLSTYLNWYQQKPGKAPKLLIYEASTLQTGVPSRFSGSRSGTDFTLTISSLQPEDFATYYCQQSYSSPPMFIFGPGTKLEIK

SEQ ID NO: 62 - protein, 108 aa, Homo sapiens
EIVMTQSPGTLSLSPGERATLSCRASQSVNSNYLAWYQQRPGQAPRLLISGASSRATDIPDRFSGSGSGTDFALIISRLETEDFAVYYCQQYGGSPPTFGGGTKVDIK

SEQ ID NO: 63 - protein, 110 aa, Homo sapiens
DIVMTQSPGTLSLSPGERATLSCRASQSVRRNYLAWYQQKPGQAPRLLIFGASSRATGIPDRFSGSGSGTDFTLTISRLEPEDFAVYYCQQYDSSPPIFTFGPGTKVEIK

SEQ ID NO: 64 - protein, 110 aa, Homo sapiens
EIVMTQSPGTLSLSPGERATLSCRASQNVRRNYLAWYQQKPGQAPRLLIYGASSRATGIPDRFSGSGSGTDFTLTISRLEPEDFVVYYCQQYDSSPPIFTFGPGTKVEIK

SEQ ID NO: 65 - protein, 111 aa, Homo sapiens
QSALTQPPSASGTPGQRVTISCSGSTSNIGRNSVNWYQQLPGTAPKLLMYSNNQRPSGVPDRFSGSKSGTSASLAISGLQSEDEADYYCAAWDDSLNGSVVFGGGTKLTVL

SEQ ID NO: 66 - protein, 109 aa, Homo sapiens
DIQMTQSPSSVSASVGDRVIITCRASQGISSWLAWYQQKPGKAPKLLIYAASILQSGVPSRFSGSGSGTDFTLTISSLQPEDFATYYCQQANSFPPIFTFGPGTKLEIK

SEQ ID NO: 67 - protein, 110 aa, Homo sapiens
EIVMTQSPGSLSLSPGESVTLSCRASQSVNSRFFAWYQQKPGQAPRLLMFGPSRRAAGIPDRFSGSGSGTDFTLTISRLEPEDFAVYYCQQYAASPPMYTFGQGTKVEIK

SEQ ID NO: 68 - protein, 110 aa, Homo sapiens
EIVLTQSPGSLSLSPGESVTLSCRASQSVNSRFFAWYQQKPGQAPRLLMFGPSSRAAGIPDRFSGSGSGTDFTLTISRLEPEDFAVYYCQQYAASPPMYTFGQGTKLEIK

SEQ ID NO: 69 - protein, 108 aa, Homo sapiens
EIVLTQSPGTLSLSPGERATLSCRASQTISDNSLAWYQQKPGQAPRLLIYGASNRVTGIPDRFSGFGSGTDFTLTISRLEPEDFAVYYCQQYGSSPPTFGQGTKLEIK

SEQ ID NO: 70 - protein, 102 aa, Homo sapiens
EIVMTQSPPTLSLSPGERATLSCRASQSVSTNLAWYQQKPGQAPRLLIYGASTRATGIPARFSGGGSGTDFTLTISSLQSEDFAVYYCGAVTFGQGTKLEIK

SEQ ID NO: 71 - protein, 110 aa, Homo sapiens
QSVLTQPPSVSAAPERKVTISCSGSSSNIGTNFVSWYQQLPGTAPKLLIYENNKRPSGIPDRFSGSKSGTSATLGITGLQTGDEADYYCGAWDSTPGTWVFGGGTRLTVL

SEQ ID NO: 72 - protein, 110 aa, Homo sapiens
QSVLTQPPSVSAAPGQRVTISCSGSSSNIGNNFVSWYQQFPGTAPKLLIYENNKRPSQIPDRFSGSKSGTSATLGITGLQTGDEADYYCGTWDTSVGAWVFGGGTKLTVL

SEQ ID NO: 73 - protein, 110 aa, Homo sapiens
QSVLTQPPSASAAPGQRVAISCSGSSSNIGSNFVSWYQHLPGTAPKLLISENDQRPSGVPDRFSGSRSGTSATLGIAGLQAGDEADYYCGTWDTSLGEWVFGGGTKLTVL

SEQ ID NO: 74 - protein, 112 aa, Homo sapiens
DIVMTQSPDSLAVSLGERATINCKSSQSILHSSNKKNYLAWYQQKPGQPPKLIIYWASTRESGVPDRFSGSGSGTDFTLTINSLQAEDVAVYYCHQYYSSRTFGQGTKLEIK

SEQ ID NO: 75 - protein, 109 aa, Homo sapiens
DIQMTQSPSSVSASVGDRVIITCRASQVISNWLAWYQQKPGKAPNLLIYAVSNLQSGVPSRFSGSGSGTNFTLTISSLQPEDFATYFCQQASGFPPLFTFGPGTKVDIK

SEQ ID NO: 76 - protein, 103 aa, Homo sapiens
DIQMTQSPSFLSASVGDRVTITCRASQGISSYLAWYQQKPGKTPKLLIYAASTLQSGVPSRFSGSGSGTEFTLTISSLQPEDFATYFCQQLVAFGQGTKLEIK

SEQ ID NO: 77 - protein, 110 aa, Homo sapiens
QSVLTQPPSVSAAPGQKVTISCSGSSSNIGENYVSWFLQLPGTAPKVVIYENNNRPSEIPDRFSGSKSGTSATLGITGLQPGDEADYYCGSWDSSLSVWVFGGGTKLTVL

SEQ ID NO: 78 - protein, 110 aa, Homo sapiens
DVVMTQSPGTLSLSPGERATLSCRASQSVRRNYLAWYQQKPGQAPRLLIYGASSRATGIPDRFSGSGSGTDFTLTISRLEPEDFAVYYCQQYDSSPPIFTFGPGTKVEIK

SEQ ID NO: 79 - protein, 110 aa, Homo sapiens
DVVMTQSPGTLSLSAGERATLSCRASQSITNNYLAWFQQKPGQAPRLLIYGASRRATGIPDRFSGSGSGTDFTLTISRLAPEDFAVYYCLQYGSSPPIFTFGPGTKVEIK

SEQ ID NO: 80 - protein, 110 aa, Homo sapiens
DIVMTQSPGTLSLSPGERATLSCRASQTITNNYLAWYQQKPGQAPRLLIYGASRRATGIPDRFSGSGSGTDFTLTISRLAPEDFAVYYCLQYGSSPPIFTFGPGTKVEIK

SEQ ID NO: 81 - protein, 8 aa, Homo sapiens
GYTFTSDY

SEQ ID NO: 82 - protein, 8 aa, Homo sapiens
VNPSGSGT

SEQ ID NO: 83 - protein, 11 aa, Homo sapiens
ASGILTGLFDY

SEQ ID NO: 84 - protein, 7 aa, Homo sapiens
QTMTKNY

SEQ ID NO: 85 - protein, 11 aa, Homo sapiens
LQYGSSPPIFT

SEQ ID NO: 86 - protein, 8 aa, Homo sapiens
GNTFTNYY

SEQ ID NO: 87 - protein, 8 aa, Homo sapiens
ISPSGDGT

SEQ ID NO: 88 - protein, 11 aa, Homo sapiens
ARGSNWGPWDY

SEQ ID NO: 89 - protein, 7 aa, Homo sapiens
QSVRRNY

SEQ ID NO: 90 - protein, 11 aa, Homo sapiens
QQYDSSPPIFT

SEQ ID NO: 91 - protein, 8 aa, Homo sapiens
GFNFGDYA

SEQ ID NO: 92 - protein, 10 aa, Homo sapiens
IRNKAYGGTP

SEQ ID NO: 93 - protein, 8 aa, Homo sapiens
ATTHAGVS

SEQ ID NO: 94 - protein, 12 aa, Homo sapiens
LTVLNPSNNKFH

SEQ ID NO: 95 - protein, 9 aa, Homo sapiens
QQYYSLPIT

SEQ ID NO: 96 - protein, 8 aa, Homo sapiens
EYAFTTYY

SEQ ID NO: 97 - protein, 8 aa, Homo sapiens
INPSGGGT

SEQ ID NO: 98 - protein, 11 aa, Homo sapiens
ASGFRGPLFDY

SEQ ID NO: 99 - protein, 7 aa, Homo sapiens
QSVTSNY

SEQ ID NO: 100 - protein, 11 aa, Homo sapiens
QQYSSSPPRLT

SEQ ID NO: 101 - protein, 8 aa, Homo sapiens
GNTFTSYY

SEQ ID NO: 102 - protein, 8 aa, Homo sapiens
INPSGDGT

SEQ ID NO: 103 - protein, 11 aa, Homo sapiens
ASGFRGPLFDY

SEQ ID NO: 104 - protein, 7 aa, Homo sapiens
QSVRKNY

SEQ ID NO: 105 - protein, 11 aa, Homo sapiens
QQYDSSPPRLT

SEQ ID NO: 106 - protein, 8 aa, Homo sapiens
GFNFGDYA

SEQ ID NO: 107 - protein, 10 aa, Homo sapiens
IRNKAYGGTP

SEQ ID NO: 108 - protein, 8 aa, Homo sapiens
ATTHAGVS

SEQ ID NO: 109 - protein, 12 aa, Homo sapiens
LTVLNPSNNKFH

SEQ ID NO: 110 - protein, 9 aa, Homo sapiens
QQYYSLPIT

SEQ ID NO: 111 - protein, 8 aa, Homo sapiens
GYIFSNYY

SEQ ID NO: 112 - protein, 8 aa, Homo sapiens
INPTGVNT

SEQ ID NO: 113 - protein, 8 aa, Homo sapiens
SRGSSPWD

SEQ ID NO: 114 - protein, 9 aa, Homo sapiens
TGAVTSGFY

SEQ ID NO: 115 - protein, 10 aa, Homo sapiens
LLYFGAAQLL

SEQ ID NO: 116 - protein, 8 aa, Homo sapiens
GFPFSNYW

SEQ ID NO: 117 - protein, 8 aa, Homo sapiens
IKRDGSEK

SEQ ID NO: 118 - protein, 7 aa, Homo sapiens
ATWFGIH

SEQ ID NO: 119 - protein, 12 aa, Homo sapiens
QSLLYSSSNKNY

SEQ ID NO: 120 - protein, 9 aa, Homo sapiens
QQHYSPPHT

SEQ ID NO: 121 - protein, 8 aa, Homo sapiens
GFTFSNYA

SEQ ID NO: 122 - protein, 8 aa, Homo sapiens
ISGGGGGT

SEQ ID NO: 123 - protein, 13 aa, Homo sapiens
AKFLQPQHLVFDY

SEQ ID NO: 124 - protein, 7 aa, Homo sapiens
QRVSNSY

SEQ ID NO: 125 - protein, 9 aa, Homo sapiens
QQYGRSPFT

SEQ ID NO: 126 - protein, 8 aa, Homo sapiens
GNDFATYY

SEQ ID NO: 127 - protein, 8 aa, Homo sapiens
INPSGDGT

SEQ ID NO: 128 - protein, 11 aa, Homo sapiens
ASGFTGPLFDY

SEQ ID NO: 129 - protein, 7 aa, Homo sapiens
QRVINRY

SEQ ID NO: 130 - protein, 11 aa, Homo sapiens
QHYASSPPRLT

SEQ ID NO: 131 - protein, 8 aa, Homo sapiens
GNTFTNYY

SEQ ID NO: 132 - protein, 8 aa, Homo sapiens
IDPSCGGT

SEQ ID NO: 133 - protein, 10 aa, Homo sapiens
AMTLYGVFDY

SEQ ID NO: 134 - protein, 7 aa, Homo sapiens
QSVRNKY

SEQ ID NO: 135 - protein, 11 aa, Homo sapiens
QQYDSSPPSFT

SEQ ID NO: 136 - protein, 8 aa, Homo sapiens
GNIFTSYY

SEQ ID NO: 137 - protein, 8 aa, Homo sapiens
INPSGDGT

SEQ ID NO: 138 - protein, 10 aa, Homo sapiens
AMTLYGVFDY

SEQ ID NO: 139 - protein, 7 aa, Homo sapiens
QSVRNNY

SEQ ID NO: 140 - protein, 11 aa, Homo sapiens
QQYDSSPPSFT

SEQ ID NO: 141 - protein, 8 aa, Homo sapiens
GFTFSNYA

SEQ ID NO: 142 - protein, 8 aa, Homo sapiens
ISGGSGAR

SEQ ID NO: 143 - protein, 13 aa, Homo sapiens
AKWTHYGDFGVDL

SEQ ID NO: 144 - protein, 7 aa, Homo sapiens
HGVSSSS

SEQ ID NO: 145 - protein, 9 aa, Homo sapiens
QQYGSPPYT

SEQ ID NO: 146 - protein, 8 aa, Homo sapiens
GYTFTSYY

SEQ ID NO: 147 - protein, 8 aa, Homo sapiens
ITPSGDNT

SEQ ID NO: 148 - protein, 13 aa, Homo sapiens
ASGDSSDSSSYHY

SEQ ID NO: 149 - protein, 6 aa, Homo sapiens
QSISRW

SEQ ID NO: 150 - protein, 11 aa, Homo sapiens
QQANSSPPSFT

SEQ ID NO: 151 - protein, 8 aa, Homo sapiens
GYNFIDHY

SEQ ID NO: 152 - protein, 8 aa, Homo sapiens
ISPSAGYT

SEQ ID NO: 153 - protein, 10 aa, Homo sapiens
ARLRFGVNDH

SEQ ID NO: 154 - protein, 7 aa, Homo sapiens
QSVRSNY

SEQ ID NO: 155 - protein, 11 aa, Homo sapiens
QQYDSSPPIFT

SEQ ID NO: 156 - protein, 10 aa, Homo sapiens
GGSISSGGYY

SEQ ID NO: 157 - protein, 7 aa, Homo sapiens
ISYRGNT

SEQ ID NO: 158 - protein, 15 aa, Homo sapiens
AGTSAGGMGSHAMDV

SEQ ID NO: 159 - protein, 8 aa, Homo sapiens
SSNIGINF

SEQ ID NO: 160 - protein, 10 aa, Homo sapiens
GAWDSSLGWV

SEQ ID NO: 161 - protein, 8 aa, Homo sapiens
GYTFTDYY

SEQ ID NO: 162 - protein, 8 aa, Homo sapiens
IKPSAGNT

SEQ ID NO: 163 - protein, 10 aa, Homo sapiens
ARGGVHGLDY

SEQ ID NO: 164 - protein, 8 aa, Homo sapiens
SSNIGNNY

SEQ ID NO: 165 - protein, 11 aa, Homo sapiens
GTWDTNLGAFV

SEQ ID NO: 166 - protein, 8 aa, Homo sapiens
GDTFASNY

SEQ ID NO: 167 - protein, 8 aa, Homo sapiens
INPSGDRT

SEQ ID NO: 168 - protein, 10 aa, Homo sapiens
ARLRFGVNDY

SEQ ID NO: 169 - protein, 7 aa, Homo sapiens
QSVTSNY

SEQ ID NO: 170 - protein, 11 aa, Homo sapiens
EQYGSSPPIFT

SEQ ID NO: 171 - protein, 8 aa, Homo sapiens
GFTFSRYA

SEQ ID NO: 172 - protein, 8 aa, Homo sapiens
TSGGGGSS

SEQ ID NO: 173 - protein, 12 aa, Homo sapiens
AKVGTTMVYFDY

SEQ ID NO: 174 - protein, 7 aa, Homo sapiens
QSVSSSH

SEQ ID NO: 175 - protein, 9 aa, Homo sapiens
QQYGDSPPT

SEQ ID NO: 176 - protein, 8 aa, Homo sapiens
GFTFSSYA

SEQ ID NO: 177 - protein, 8 aa, Homo sapiens
ISGASGSS

SEQ ID NO: 178 - protein, 11 aa, Homo sapiens
AKVKQVLYFDY

SEQ ID NO: 179 - protein, 6 aa, Homo sapiens
QSVSSS

SEQ ID NO: 180 - protein, 9 aa, Homo sapiens
QQYGSSPPT

SEQ ID NO: 181 - protein, 8 aa, Homo sapiens
GFTFSNYY

SEQ ID NO: 182 - protein, 8 aa, Homo sapiens
INPSGQST

SEQ ID NO: 183 - protein, 10 aa, Homo sapiens
VRVARGGFDI

SEQ ID NO: 184 - protein, 6 aa, Homo sapiens
QSLSTY

SEQ ID NO: 185 - protein, 11 aa, Homo sapiens
QQSYSSPPMFI

SEQ ID NO: 186 - protein, 8 aa, Homo sapiens
GLTFSRHA

SEQ ID NO: 187 - protein, 8 aa, Homo sapiens
ISGGGGGT

SEQ ID NO: 188 - protein, 12 aa, Homo sapiens
AKVAGGTVFFDY

SEQ ID NO: 189 - protein, 7 aa, Homo sapiens
QSVNSNY

SEQ ID NO: 190 - protein, 9 aa, Homo sapiens
QQYGGSPPT

SEQ ID NO: 191 - protein, 8 aa, Homo sapiens
GDTFTNYY

SEQ ID NO: 192 - protein, 8 aa, Homo sapiens
INPSGYGT

SEQ ID NO: 193 - protein, 11 aa, Homo sapiens
ARGSNWGPWDY

SEQ ID NO: 194 - protein, 7 aa, Homo sapiens
QSVRRNY

SEQ ID NO: 195 - protein, 11 aa, Homo sapiens
QQYDSSPPIFT

SEQ ID NO: 196 - protein, 8 aa, Homo sapiens
GTTFTSDY

SEQ ID NO: 197 - protein, 8 aa, Homo sapiens
IDPSGGGT

SEQ ID NO: 198 - protein, 9 aa, Homo sapiens
ARGSSGWYF

SEQ ID NO: 199 - protein, 7 aa, Homo sapiens
QNVRRNY

SEQ ID NO: 200 - protein, 11 aa, Homo sapiens
QQYDSSPPIFT

SEQ ID NO: 201 - protein, 8 aa, Homo sapiens
GYTFTNYY

SEQ ID NO: 202 - protein, 8 aa, Homo sapiens
IKPSGGNT

SEQ ID NO: 203 - protein, 12 aa, Homo sapiens
ANTMIRGIIITH

SEQ ID NO: 204 - protein, 8 aa, Homo sapiens
TSNIGRNS

SEQ ID NO: 205 - protein, 12 aa, Homo sapiens
AAWDDSLNGSVV

SEQ ID NO: 206 - protein, 8 aa, Homo sapiens
GYTFTTYY

SEQ ID NO: 207 - protein, 8 aa, Homo sapiens
INPSGAGT

SEQ ID NO: 208 - protein, 6 aa, Homo sapiens
QGISSW

SEQ ID NO: 209 - protein, 11 aa, Homo sapiens
QQANSFPPIFT

SEQ ID NO: 210 - protein, 13 aa, Homo sapiens
ASPPRGSSSALGV

SEQ ID NO: 211 - protein, 8 aa, Homo sapiens
ADTLSGYY

SEQ ID NO: 212 - protein, 8 aa, Homo sapiens
ITPNGAGT

SEQ ID NO: 213 - protein, 11 aa, Homo sapiens
ARGEDNGSFFL

SEQ ID NO: 214 - protein, 7 aa, Homo sapiens
QSVNSRF

SEQ ID NO: 215 - protein, 11 aa, Homo sapiens
QQYAASPPMYT

SEQ ID NO: 216 - protein, 8 aa, Homo sapiens
GSTLSGYY

SEQ ID NO: 217 - protein, 8 aa, Homo sapiens
INPSGTGT

SEQ ID NO: 218 - protein, 11 aa, Homo sapiens
ARMEDNGSYFL

SEQ ID NO: 219 - protein, 7 aa, Homo sapiens
QSVNSRF

SEQ ID NO: 220 - protein, 11 aa, Homo sapiens
QQYAASPPMYT

SEQ ID NO: 221 - protein, 8 aa, Homo sapiens
GFTFSNYA

SEQ ID NO: 222 - protein, 8 aa, Homo sapiens
ISGRGGAT

SEQ ID NO: 223 - protein, 11 aa, Homo sapiens
AKLLMVMVFDH

SEQ ID NO: 224 - protein, 7 aa, Homo sapiens
QTISDNS

SEQ ID NO: 225 - protein, 9 aa, Homo sapiens
QQYGSSPPT

SEQ ID NO: 226 - protein, 10 aa, Homo sapiens
GASINSGRYY

SEQ ID NO: 227 - protein, 7 aa, Homo sapiens
IHYTGST

SEQ ID NO: 228 - protein, 25 aa, Homo sapiens
ASLWRESCTTAGCYPKDTSLYYFDY

SEQ ID NO: 229 - protein, 6 aa, Homo sapiens
QSVSTN

SEQ ID NO: 230 - protein, 4 aa, Homo sapiens
GAVT

SEQ ID NO: 231 - protein, 8 aa, Homo sapiens
GYTFTDSY

SEQ ID NO: 232 - protein, 8 aa, Homo sapiens
IKPSGGNT

SEQ ID NO: 233 - protein, 10 aa, Homo sapiens
ARDSRGPGIF

SEQ ID NO: 234 - protein, 8 aa, Homo sapiens
SSNIGTNF

SEQ ID NO: 235 - protein, 11 aa, Homo sapiens
GAWDSTPGTWV

SEQ ID NO: 236 - protein, 8 aa, Homo sapiens
GYTFSSSY

SEQ ID NO: 237 - protein, 8 aa, Homo sapiens
IKPSGGNT

SEQ ID NO: 238 - protein, 10 aa, Homo sapiens
AGDLRGVGGS

SEQ ID NO: 239 - protein, 8 aa, Homo sapiens
SSNIGNNF

SEQ ID NO: 240 - protein, 11 aa, Homo sapiens
GTWDTSVGAWV

SEQ ID NO: 241 - protein, 8 aa, Homo sapiens
GYTLTDYY

SEQ ID NO: 242 - protein, 8 aa, Homo sapiens
IKPSGGNT

SEQ ID NO: 243 - protein, 10 aa, Homo sapiens
ARGGRHAHDI

SEQ ID NO: 244 - protein, 8 aa, Homo sapiens
SSNIGSNF

SEQ ID NO: 245 - protein, 11 aa, Homo sapiens
GTWDTSLGEWV

SEQ ID NO: 246 - protein, 8 aa, Homo sapiens
GLPFSGAW

SEQ ID NO: 247 - protein, 10 aa, Homo sapiens
IKSKSDGGTI

SEQ ID NO: 248 - protein, 6 aa, Homo sapiens
NWNLDY

SEQ ID NO: 249 - protein, 12 aa, Homo sapiens
QSILHSSNKKNY

SEQ ID NO: 250 - protein, 8 aa, Homo sapiens
HQYYSSRT

SEQ ID NO: 251 - protein, 8 aa, Homo sapiens
GFTFSDYY

SEQ ID NO: 252 - protein, 8 aa, Homo sapiens
INPSGTGT

SEQ ID NO: 253 - protein, 10 aa, Homo sapiens
ARVVRGASSL

SEQ ID NO: 254 - protein, 6 aa, Homo sapiens
QVISNW

SEQ ID NO: 255 - protein, 11 aa, Homo sapiens
QQASGFPPLFT

SEQ ID NO: 256 - protein, 10 aa, Homo sapiens
GGSISSGGYY

SEQ ID NO: 257 - protein, 7 aa, Homo sapiens
IHYSGST

SEQ ID NO: 258 - protein, 20 aa, Homo sapiens
ARGFRENYDNSGYSSYYFDY

SEQ ID NO: 259 - protein, 6 aa, Homo sapiens
QGISSY

SEQ ID NO: 260 - protein, 5 aa, Homo sapiens
QQLVA

SEQ ID NO: 261 - protein, 8 aa, Homo sapiens
GYTFTNFY

SEQ ID NO: 262 - protein, 8 aa, Homo sapiens
ISTSAGST

SEQ ID NO: 263 - protein, 12 aa, Homo sapiens
ARDLYGSRNFHY

SEQ ID NO: 264 - protein, 8 aa, Homo sapiens
SSNIGENY

SEQ ID NO: 265 - protein, 11 aa, Homo sapiens
GSWDSSLSVWV

SEQ ID NO: 266 - protein, 8 aa, Homo sapiens
GTTFTSDY

SEQ ID NO: 267 - protein, 8 aa, Homo sapiens
IDPSGGGT

SEQ ID NO: 268 - protein, 9 aa, Homo sapiens
ARGSSGWYF

SEQ ID NO: 269 - protein, 7 aa, Homo sapiens
QSVRRNY

SEQ ID NO: 270 - protein, 11 aa, Homo sapiens
QQYDSSPPIFT

SEQ ID NO: 271 - protein, 8 aa, Homo sapiens
GYTFTSDY

SEQ ID NO: 272 - protein, 8 aa, Homo sapiens
INPSGSGT

SEQ ID NO: 273 - protein, 11 aa, Homo sapiens
ASGILTGLFDY

SEQ ID NO: 274 - protein, 7 aa, Homo sapiens
QSITNNY

SEQ ID NO: 275 - protein, 11 aa, Homo sapiens
LQYGSSPPIFT

SEQ ID NO: 276 - protein, 8 aa, Homo sapiens
EYTFTNDY

SEQ ID NO: 277 - protein, 8 aa, Homo sapiens
INPSGSGT

SEQ ID NO: 278 - protein, 11 aa, Homo sapiens
ASGILTGLFDY

SEQ ID NO: 279 - protein, 7 aa, Homo sapiens
QTITNNY

SEQ ID NO: 280 - protein, 11 aa, Homo sapiens
LQYGSSPPIFT

SEQ ID NO: 281 - protein, 25 aa, synthetic construct
PLQPELDSFKEELDKYFKNHTSPDV

SEQ ID NO: 282 - protein, 25 aa, synthetic construct
PLQPELDSFKEELDKYFKNHTSPDV

SEQ ID NO: 283 - protein, 25 aa, synthetic construct
PLQPELESFKQELDKYFKNHTSPDV

SEQ ID NO: 284 - protein, 25 aa, synthetic construct
PLQPELDSFKEELDKYFKNHTSPDV

SEQ ID NO: 285 - protein, 27 aa, synthetic construct
PLLGNSTGLDFQDELDEFFKNVSTSIP

SEQ ID NO: 286 - protein, 26 aa, synthetic construct
HSVPKLSDFFESELSHWFKNQTSIAP

SEQ ID NO: 287 - protein, 25 aa, synthetic construct
TSIPNLDDFKEELDQWFKNQTSVAP

SEQ ID NO: 288 - protein, 25 aa, synthetic construct
NSIPNLPDFKEELDKWFKNQTSIAP

(19) United States
(12) Patent Application Publication
(10) Pub. No.: US 2026/0035438 A1
(43) Pub. Date: Feb. 5, 2026

(54) HUMAN BROADLY NEUTRALIZING ANTIBODIES AGAINST BETACORONAVIRUSES

(71) Applicant: The Scripps Research Institute, La Jolla, CA (US)

(72) Inventors: Panpan Zhou, San Diego, CA (US); Wanting He, San Diego, CA (US); Ge Song, San Diego,, CA (US); Dennis R. Burton, La Jolla, CA (US); Raiees Andrabi, San Diego, CA (US)

(21) Appl. No.: 18/842,872

(22) PCT Filed: Feb. 27, 2023
(86) PCT No.: PCT/US23/63308

§ 371 Date: Aug. 30, 2024

Related U.S. Application Data
(60) Provisional application No. 63/316,565, filed on Mar. 4, 2022.

(51) Int. Cl. C07K 16/10; A61K 39/00; A61K 47/68; A61P 31/14; G01N 33/569

(52) U.S. Cl. CPC C07K 16/1003; A61K 47/6841; A61P 31/14; G01N 33/56983; A61K 2039/505; C07K 2317/52; C07K 2317/565; C07K 2317/567; C07K 2317/76; G01N 2333/165

(57) ABSTRACT

The invention provides novel broadly neutralizing antibodies and related antibody compositions against betacoronaviruses, e.g., SARS-CoV-2. Also provided in the invention are polynucleotides and vectors encoding such antibodies, as well as pharmaceutical compositions containing the antibodies or polynucleotides. Therapeutic uses of the antibodies or pharmaceutical compositions in preventing or treating betacoronaviral infections (e.g., SARS-CoV-2 infection) are also encompassed by the invention.

### CROSS-REFERENCE TO RELATED APPLICATIONS

The subject patent application is a § 371 U.S. national phase filing of International Patent Application No. PCT/US23/63308 (filed Feb. 27, 2023; now expired), which claims the benefit of priority to U.S. Provisional Patent Application No. 63/316,565 (filed Mar. 4, 2022; now expired). The full disclosure of the priority application is incorporated herein by reference in its entirety and for all purposes.

### STATEMENT OF GOVERNMENT SUPPORT

This invention was made with government support under grant numbers AI144462 and AI161818 awarded by the National Institutes of Health. The government has certain rights in the invention.

### SEQUENCE LISTING

The instant application contains a Sequence Listing, which is submitted in .xml format and is hereby incorporated by reference in its entirety. Said .xml file is named “2137_1PC_SeqListing”, was created on Jun. 20, 2023, and is 252 KB bytes in size.

### BACKGROUND OF THE INVENTION

The initial successes of SARS-CoV-2 vaccines were due in part to the relative ease of inducing protective neutralizing antibodies (nAbs) to immunodominant epitopes on the receptor binding domain (RBD) of the S1 subunit of the spike protein (1-5). The most potent nAbs target epitopes overlapping the ACE2 receptor binding site (RBS) (RBS-A/class 1; RBS-B,C,D/class 2 antibodies) and require little affinity maturation to neutralize at very low (single digit ng/ml) concentrations (3, 6-12). However, mutations, particularly in and around the RBS, readily generate viral variants that are resistant to neutralization by commonly induced classes of antibodies (13, 14) without significantly negatively impacting viral fitness, and have led to several Variants of Concern (VOCs) (14-25). For example, the K417N/T and E484K mutations in the Beta and Gamma VOCs lead to neutralization escape from the vast majority of RBS-A/class 1 and RBS-B, C, D/class 2 nAbs (12-14). Other sarbecoviruses using ACE2 as receptor, such as SARS-CoV-1, and betacoronaviruses using receptors other than ACE2, such as MERS-CoV, show even more sequence divergence in the RBS region (26-28). The emergence of SARS-CoV-2 VOCs, together with a desire to have the capability to respond to novel coronaviruses with pandemic potential, has focused effort on vaccines and antibodies that target the most conserved regions of the spike protein (29-31). The “lower” more conserved faces of the RBD have been investigated and are targeted by many nAbs with greater breadth of neutralization against SARS-CoV-2 variants and diverse sarbecoviruses than for example RBS-A/class 1 or RBS-B,C,D/class 2 nAbs (32-41). However, the Omicron variant has demonstrated escape also from some nAbs targeting these more conserved regions of the RBD (19, 25).

There is a strong need for nAbs targeting other epitopes of the coronavirus spike, as well as related SARS-CoV-2 vaccines against VOCs and, more ambitiously, pan-betacoronavirus vaccines (29-31). The present invention is directed to addressing such unmet needs in the art.

### SUMMARY OF THE INVENTION

In one aspect, the invention provides pharmaceutical compositions that contain a therapeutically effective amount of an antibody or antigen-binding fragment thereof that specifically binds to the S2 stem helix of a betacoronavirus, and a pharmaceutically acceptable carrier. In various embodiments, the S2 stem helix targeting antibody or antigen-binding fragment has heavy chain CDR (HCDR) and/or light chain CDR (LCDR) sequences as that of the bnAbs exemplified herein (e.g., Table 1). The HCDR1-3 sequences of the exemplified antibodies are set forth respectively in (1) SEQ ID NOs:81-83, (2) SEQ ID NOs:86-88, (3) SEQ ID NOs:91-93, (4) SEQ ID NOs:96-98, (5) SEQ ID NOs:101-103, (6) SEQ ID NOs:106-108, (7) SEQ ID NOs:111-113, (8) SEQ ID NOs:116-118, (9) SEQ ID NOs:121-123, (10) SEQ ID NOs:126-128, (11) SEQ ID NOs:131-133, (12) SEQ ID NOs:136-138, (13) SEQ ID NOs:141-143, (14) SEQ ID NOs:146-148, (15) SEQ ID NOs:151-153, (16) SEQ ID NOs:156-158, (17) SEQ ID NOs:161-163, (18) SEQ ID NOs:166-168, (19) SEQ ID NOs:171-173, (20) SEQ ID NOs:176-178, (21) SEQ ID NOs:181-183, (22) SEQ ID NOs:186-188, (23) SEQ ID NOs:191-193, (24) SEQ ID NOs:196-198, (25) SEQ ID NOs:201-203, (26) SEQ ID NOs:206-208, (27) SEQ ID NOs:211-213, (28) SEQ ID NOs:216-218, (29) SEQ ID NOs:221-223, (30) SEQ ID NOs:226-228, (31) SEQ ID NOs:231-233, (32) SEQ ID NOs:236-238, (33) SEQ ID NOs:241-243, (34) SEQ ID NOs:246-248, (35) SEQ ID NOs:251-253, (36) SEQ ID NOs:256-258, (37) SEQ ID NOs:261-263, (38) SEQ ID NOs:266-268, (39) SEQ ID NOs:271-273, or (40) SEQ ID NOs:276-278. The LCDR1-3 sequences of the exemplified antibodies are set forth respectively in (1) SEQ ID NO:84, GAS, and SEQ ID NO:85, (2) SEQ ID NO:89, GAS, and SEQ ID NO:90, (3) SEQ ID NO:94, WAS, and SEQ ID NO: 95, (4) SEQ ID NO:99, GAS, and SEQ ID NO:100, (5) SEQ ID NO:104, GAS, and SEQ ID NO:105, (6) SEQ ID NO:109, WAS, and SEQ ID NO:110, (7) SEQ ID NO:114, SSY, and SEQ ID NO:115, (8) SEQ ID NO:119, YAS, and SEQ ID NO:120, (9) SEQ ID NO:124, GAS, and SEQ ID NO:125, (10) SEQ ID NO:129, GAS, and SEQ ID NO:130, (11) SEQ ID NO:134, GAS, and SEQ ID NO:135, (12) SEQ ID NO:139, GAS, and SEQ ID NO:140, (13) SEQ ID NO:144, DAS, and SEQ ID NO:145, (14) SEQ ID NO:149, AVS, and SEQ ID NO:150, (15) SEQ ID NO:154, GVS, and SEQ ID NO:155, (16) SEQ ID NO:159, DNN, and SEQ ID NO:160, (17) SEQ ID NO:164, ENN, and SEQ ID NO:165, (18) SEQ ID NO:169, GAS, and SEQ ID NO:170, (19) SEQ ID NO:174, GAS, and SEQ ID NO:175, (20) SEQ ID NO:179, DAS, and SEQ ID NO:180, (21) SEQ ID NO:184, EAS, and SEQ ID NO:185, (22) SEQ ID NO:189, GAS, and SEQ ID NO:190, (23) SEQ ID NO:194, GAS, and SEQ ID NO:195, (24) SEQ ID NO:199, GAS, and SEQ ID NO:200, (25) SEQ ID NO:204, SNN, and SEQ ID NO:205, (26) SEQ ID NO:209, AAS, and SEQ ID NO:210, (27) SEQ ID NO:214, GPS, and SEQ ID NO:215, (28) SEQ ID NO:219, GPS, and SEQ ID NO:220, (29) SEQ ID NO:224, GAS, and SEQ ID NO:225, (30) SEQ ID NO:229, GAS, and SEQ ID NO:230, (31) SEQ ID NO:234, ENN, and SEQ ID NO:235, (32) SEQ ID NO:239, ENN, and SEQ ID NO:240, (33) SEQ ID NO:244, END, and SEQ ID NO:245, (34) SEQ ID NO:249, WAS, and SEQ ID NO:250, (35) SEQ ID NO:254, AVS, and SEQ ID NO:255, (36) SEQ ID NO:259, AAS, and SEQ ID NO:260, (37) SEQ ID NO:264, ENN, and SEQ ID NO:265, (38) SEQ ID NO:269, GAS, and SEQ ID NO:270, (39) SEQ ID NO:274, GAS, and SEQ ID NO:275, or (40) SEQ ID NO:279, GAS, and SEQ ID NO:280.

In some embodiments, the S2 stem helix targeting antibody or antigen-binding fragment has heavy chain LHCDR and LCDR sequences set forth respectively in (1) SEQ ID NOs:81-84, GAS, and SEQ ID NO:85; (2) SEQ ID NOs:86-89, GAS, and SEQ ID NO:90; (3) SEQ ID NOs:91-94, WAS, and SEQ ID NO:95; (4) SEQ ID NOs:96-99, GAS, and SEQ ID NO:100; (5) SEQ ID NOs:101-104, GAS, and SEQ ID NO:105; (6) SEQ ID NOs:106-109, WAS, and SEQ ID NO:110; (7) SEQ ID NOs:111-114, SSY, and SEQ ID NO:115; (8) SEQ ID NOs:116-119, YAS, and SEQ ID NO:120; (9) SEQ ID NOs:121-124, GAS, and SEQ ID NO:125; (10) SEQ ID NOs:126-129, GAS, and SEQ ID NO: 130; (11) SEQ ID NOs:131-134, GAS, and SEQ ID NO:135; (12) SEQ ID NOs:136-139, GAS, and SEQ ID NO:140; (13) SEQ ID NOs:141-144, DAS, and SEQ ID NO:145; (14) SEQ ID NOs:146-149, AVS, and SEQ ID NO:150; (15) SEQ ID NOs:151-154, GVS, and SEQ ID NO:155; (16) SEQ ID NOs:156-159, DNN, and SEQ ID NO:160; (17) SEQ ID NOs:161-164, ENN, and SEQ ID NO:165; (18) SEQ ID NOs:166-169, GAS, and SEQ ID NO:170; (19) SEQ ID NOs:171-174, GAS, and SEQ ID NO:175; (20) SEQ ID NOs:176-179, DAS, and SEQ ID NO:180; (21) SEQ ID NOs:181-184, EAS, and SEQ ID NO:185; (22) SEQ ID NOs:186-189, GAS, and SEQ ID NO:190; (23) SEQ ID NOs:191-194, GAS, and SEQ ID NO:195; (24) SEQ ID NOs:196-199, GAS, and SEQ ID NO:200; (25) SEQ ID NOs:201-204, SNN, and SEQ ID NO:205; (26) SEQ ID NOs:206-209, AAS, and SEQ ID NO:210; (27) SEQ ID NOs:211-214, GPS, and SEQ ID NO:215; (28) SEQ ID NOs:216-219, GPS, and SEQ ID NO:220; (29) SEQ ID NOs:221-224, GAS, and SEQ ID NO:225; (30) SEQ ID NOs:226-229, GAS, and SEQ ID NO:230; (31) SEQ ID NOs:231-234, ENN, and SEQ ID NO:235; (32) SEQ ID NOs:236-239, ENN, and SEQ ID NO:240; (33) SEQ ID NOs:241-244, END, and SEQ ID NO:245; (34) SEQ ID NOs:246-249, WAS, and SEQ ID NO:250; (35) SEQ ID NOs:251-254, AVS, and SEQ ID NO:255; (36) SEQ ID NOs:256-259, AAS, and SEQ ID NO:260; (37) SEQ ID NOs:261-264, ENN, and SEQ ID NO:265; (38) SEQ ID NOs:266-269, GAS, and SEQ ID NO:270; (39) SEQ ID NOs:271-274, GAS, and SEQ ID NO:275; or (40) SEQ ID NOs:276-279, GAS, and SEQ ID NO:280.

In some embodiments of the invention, the S2 stem helix targeting antibody or antigen-binding fragment has heavy chain variable region and light chain variable region sequences that are at least 95% identical, respectively, to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

In some embodiments, the S2 stem helix targeting antibody or antigen-binding fragment has heavy chain variable region and light chain variable region sequences that are respectively identical to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

In another aspect, the invention provides antibodies or antigen-binding fragments thereof that specifically bind to the S2 stem helix of a betacoronavirus. These antibodies contain heavy chain and light chain variable region sequences that, except for substitutions of one or more amino acid residues in the heavy chain framework region and/or the light chain framework region, are respectively identical to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

In another related aspect, the invention provides antibodies or antigen-binding fragments thereof that specifically bind to the S2 stem helix of a betacoronavirus. These antibodies contain (a) one or more non-natural amino acid residues in the Fc domain, and (b) heavy chain and light chain variable regions containing the HCDR and LCDR sequences (HCDR1-3) that are identical to one of the specific antibodies exemplified herein (e.g., Table 1). Related pharmaceutical compositions or kits that contain such a modified S2 stem helix targeting antibody are also provided in the invention.

In another aspect, the invention provides fusion compounds or fusion molecules that contain (i) an antibody or antigen-binding fragment thereof described herein that specifically binds to the S2 stem helix of a betacoronavirus, and (ii) a second moiety that is fused or conjugated to the antibody or antigen-binding fragment thereof. In some of these embodiments, the second moiety is a polypeptide. In some other embodiments, the second moiety is a small organic molecule. In some embodiments, the second moiety is a drug.

In still another aspect, the invention provides polynucleotide molecules that encode a S2 stem helix targeting antibody chain or antigen-binding fragment. In various embodiments, the polynucleotide encodes an antibody heavy chain variable region that contains HCDR1-3 that are respectively identical to (1) SEQ ID NOs:81-83, (2) SEQ ID NOs:86-88, (3) SEQ ID NOs:91-93, (4) SEQ ID NOs:96-98, (5) SEQ ID NOs:101-103, (6) SEQ ID NOs:106-108, (7) SEQ ID NOs:111-113, (8) SEQ ID NOs:116-118, (9) SEQ ID NOs:121-123, (10) SEQ ID NOs:126-128, (11) SEQ ID NOs:131-133, (12) SEQ ID NOs:136-138, (13) SEQ ID NOs:141-143, (14) SEQ ID NOs:146-148, (15) SEQ ID NOs:151-153, (16) SEQ ID NOs:156-158, (17) SEQ ID NOs:161-163, (18) SEQ ID NOs:166-168, (19) SEQ ID NOs:171-173, (20) SEQ ID NOs:176-178, (21) SEQ ID NOs:181-183, (22) SEQ ID NOs:186-188, (23) SEQ ID NOs:191-193, (24) SEQ ID NOs:196-198, (25) SEQ ID NOs:201-203, (26) SEQ ID NOs:206-208, (27) SEQ ID NOs:211-213, (28) SEQ ID NOs:216-218, (29) SEQ ID NOs:221-223, (30) SEQ ID NOs:226-228, (31) SEQ ID NOs:231-233, (32) SEQ ID NOs:236-238, (33) SEQ ID NOs:241-243, (34) SEQ ID NOs:246-248, (35) SEQ ID NOs:251-253, (36) SEQ ID NOs:256-258, (37) SEQ ID NOs:261-263, (38) SEQ ID NOs:266-268, (39) SEQ ID NOs:271-273, or (40) SEQ ID NOs:276-278. Additionally or alternatively, the polynucleotide encodes an antibody light chain variable region that contains LCDR1-3 that are respectively identical to (1) SEQ ID NO:84, GAS, and SEQ ID NO:85, (2) SEQ ID NO:89, GAS, and SEQ ID NO:90, (3) SEQ ID NO:94, WAS, and SEQ ID NO:95, (4) SEQ ID NO:99, GAS, and SEQ ID NO:100, (5) SEQ ID NO:104, GAS, and SEQ ID NO:105, (6) SEQ ID NO:109, WAS, and SEQ ID NO:110, (7) SEQ ID NO:114, SSY, and SEQ ID NO:115, (8) SEQ ID NO:119, YAS, and SEQ ID NO:120, (9) SEQ ID NO:124, GAS, and SEQ ID NO:125, (10) SEQ ID NO:129, GAS, and SEQ ID NO:130, (11) SEQ ID NO:134, GAS, and SEQ ID NO:135, (12) SEQ ID NO:139, GAS, and SEQ ID NO:140, (13) SEQ ID NO:144, DAS, and SEQ ID NO:145, (14) SEQ ID NO:149, AVS, and SEQ ID NO:150, (15) SEQ ID NO:154, GVS, and SEQ ID NO:155, (16) SEQ ID NO:159, DNN, and SEQ ID NO:160, (17) SEQ ID NO:164, ENN, and SEQ ID NO:165, (18) SEQ ID NO:169, GAS, and SEQ ID NO:170, (19) SEQ ID NO:174, GAS, and SEQ ID NO:175, (20) SEQ ID NO:179, DAS, and SEQ ID NO:180, (21) SEQ ID NO:184, EAS, and SEQ ID NO:185, (22) SEQ ID NO:189, GAS, and SEQ ID NO:190, (23) SEQ ID NO:194, GAS, and SEQ ID NO:195, (24) SEQ ID NO:199, GAS, and SEQ ID NO:200, (25) SEQ ID NO:204, SNN, and SEQ ID NO:205, (26) SEQ ID NO:209, AAS, and SEQ ID NO:210, (27) SEQ ID NO:214, GPS, and SEQ ID NO:215, (28) SEQ ID NO:219, GPS, and SEQ ID NO:220, (29) SEQ ID NO:224, GAS, and SEQ ID NO:225, (30) SEQ ID NO:229, GAS, and SEQ ID NO:230, (31) SEQ ID NO:234, ENN, and SEQ ID NO:235, (32) SEQ ID NO:239, ENN, and SEQ ID NO:240, (33) SEQ ID NO:244, END, and SEQ ID NO:245, (34) SEQ ID NO:249, WAS, and SEQ ID NO:250, (35) SEQ ID NO:254, AVS, and SEQ ID NO:255, (36) SEQ ID NO:259, AAS, and SEQ ID NO:260, (37) SEQ ID NO:264, ENN, and SEQ ID NO:265, (38) SEQ ID NO:269, GAS, and SEQ ID NO:270, (39) SEQ ID NO:274, GAS, and SEQ ID NO:275, or (40) SEQ ID NO:279, GAS, and SEQ ID NO:280.

Some polynucleotides of the invention encode a heavy chain variable region and/or a light chain variable region sequences that are at least 95% identical, respectively, to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80. In some embodiments, the polynucleotide encodes a heavy chain variable region and/or a light chain variable region sequences that are respectively identical to one of these sequence pairs. Some of the polynucleotides of the invention are cDNA molecule. In some other related embodiments, the invention provides vectors that contain one of the polynucleotides described herein, as well as host cells that harbor one or mor of these vectors. In another aspect, the invention provides methods of producing an antibody or antigen-binding fragment thereof that specifically binds to the S2 stem helix of a betacoronavirus. Typically, these methods entail recombinant expression of one or more of the vectors in a suitable host cell.

In still another aspect, the invention provides methods of treating or ameliorating symptoms associated with betacoronavirus infections in a subject. These methods involve administering to the subject, who is afflicted with infection by one or more betacoronaviruses, a pharmaceutical composition that contains a S2 stem helix binding antibody or antigen-binding fragment described herein. In some embodiments, the infection to be treated in the subject is SARS-CoV-2 infection.

In still another aspect, the invention provides methods of diagnosing a betacoronavirus infection in a subject. These methods entail (a) obtaining a biological sample from the subject, and (b) contacting the sample with a S2 stem helix binding antibody or antigen-binding fragment described herein. A detection of a specific binding between an antigen in the sample and the antibody or antigen-binding fragment indicates a positive diagnosis of a betacoronavirus infection in the subject. In some methods, the biological sample obtained from the subject for the diagnosis is a blood sample or a saliva sample.

A further understanding of the nature and advantages of the present invention may be realized by reference to the remaining portions of the specification and claims.

### DESCRIPTION OF THE DRAWINGS

FIG. 1. Binding and neutralization properties of S2 stem-helix mAbs. a. Dot plots showing ELISA binding (OD₄₀₅) reactivity of immune sera from COVID-19 convalescent donors (n=15), spike mRNA-vaccinated donors (n=10) and SARS-CoV-2 recovered-vaccinated donors (n=15) to 25-mer peptides corresponding to spike S2 stem-helix regions of human β-(sarbecoviruses: SARS-CoV-1 or 2; merbecovirus: MERS-CoV; embecoviruses: HCoV-HKU1, HCoV-OC43) and α-(HCoV-NL63 and HCoV-229E) coronaviruses. 12 out of 15 (80%) SARS-CoV-2 recovered-vaccinated donor sera show cross-reactive binding to β-CoV spike stem-helix peptides. b. Correlation between binding of infected-vaccinated sera to SARS-CoV-2 stem-helix peptide and the other β-CoV (MERS-CoV, HCoV-HKU1 and HCoV-OC43) stem-helix peptides. Responses for binding to two stem-helix peptides were compared by nonparametric Spearman correlation two-tailed test with 95% confidence interval and the Spearman correlation coefficient (r) and the p-value are indicated. c. A total of 40 S2 stem-helix mAbs were isolated from 9 SARS-CoV-2 recovered-vaccinated donors (CC9, CC24, CC25, CC67, CC68, CC84, CC92, CC95 and CC99). MAbs were isolated by single B cell sorting using SARS-CoV-2 and MERS-CoV S-proteins as baits. Heatmap showing IGVH germline gene usage (VH1-46, VH3-23 and other V-genes), lineage information (unique and expanded lineages) and V-gene nucleotide somatic hypermutations (SHMs). EC₅₀ ELISA binding titers of mAbs with β- and α-HCoV spike S2 stem-helix region peptides. MAbs showed binding to β- but not α-HCoV derived stem-helix peptides. IC₅₀ neutralization of mAbs against pseudoviruses of clade1a (SARS-CoV-2 and Pang17), clade 1b (SARS-CoV-1, WIV1, SHC014) sarbecoviruses and MERS-CoV. Spike S2 stem-helix bnAbs, CC40.8, S2P6 and CV3-25 were used as controls for binding and neutralization assays. d. 32 of 40 stem-helix bnAbs were unique clones that neutralized all ACE2-utilizing sarbecoviruses and 23 out of 32 unique mAb neutralized MERS-CoV, in addition to sarbecoviruses.

FIG. 2. Neutralization of SARS-CoV-2 VOCs, and immunogenetic properties of S2 β-CoV spike stem-helix bnAbs. a. Neutralization of 10 select S2 stem-helix bnAbs against SARS-CoV-2 (WT) and five major SARS-CoV-2 variants of concern [B.1.1.7 (Alpha), B.1.351 (Beta), P.1 (Gamma), B.1.617.2 (Delta), and B.1.1.529 (Omicron)]. b-c. Pie plots showing IGHV and IGKV/IGLV gene usage distribution of isolated stem-helix mAbs. Shown are enriched heavy chain (IGHV1-46 and IGHV3-23) (b) and light chain (IGKV3-20 and IGLV1-51) (c) gene families. Dot plots showing % nucleotide mutations (SHMs) in the heavy (VH) or light (VL) chains of isolated stem-helix mAbs. The mAbs are grouped by neutralization against sarbecoviruses or sarbecoviruses+MERS-CoV. d-e. CDRH3 (d) or CDRL3 (e) length distributions of isolated mAbs across sarbecovirus broadly neutralizing and sarbecovirus+MERS-CoV broadly neutralizing mAb groups compared to human baseline germline reference. MAbs with 10- and 11-amino acid-CDRH3 s or mAbs with 9- and 11-amino CDRL3 s, enriched in S2 stem-helix bnAbs compared to baseline germline reference, are indicated by arrows. f. Sequence conservation logos of 9 (n=8) and 11 (n=18) amino acid long CDRL3-bearing stem-helix bnAbs show enrichment of certain J-gene encoded residues.

FIG. 3. Binding kinetics and fine epitope specificities of S2 β-CoV spike stem-helix bnAbs. a. BLI binding kinetics of S2 stem-helix mature bnAbs and their inferred germline (iGL) versions to monomeric SARS-CoV-2 and MERS-CoV stem-helix peptides. Binding kinetics were obtained using a 1:1 binding kinetics fitting model on ForteBio Data Analysis software and maximum binding responses, dissociations constants (K_{D}) and on-rate (kₒₙ) and off-rate constants (k_{off}) for each antibody-protein interaction are compared. K_{D}, kₒₙ and k_{off} values were calculated only for antibody-antigen interactions where a maximum binding response of 0.2 nm was obtained. The S2 stem-helix bnAb iGL Abs showed substantially reduced binding to stem-helix peptides compared to their corresponding mature versions. Statistical comparisons between two groups were performed using a Mann-Whitney two-tailed test, (***p<0.001, ****p<0.0001; ns-p>0.05). b. Association of S2 stem-helix peptide binding by S2 bnAbs and their iGLs with CDRH3 and CDRL3 motifs. The top line shows characteristics of mAbs with and without RG motifs in CDRH3 s. The bottom line shows characteristics of mAbs with and without WD motifs in CDRL3 s. The left column shows the numbers of mAbs with and without CDR3 motifs with respect to two most common V genes: IGHV1-46 and IGHV3-23 for RG motifs in CDRH3, IGKV3-20 and IGLV1-51 for WD motifs in CDRL3 s. The middle and right columns show the responses of iGL and mature mAbs to stem peptides of SARS-CoV-2 and MERS-CoV, respectively. P-values of associations between RG/WD motifs and the responses are shown on tops of the plots and denoted as follows: ns≥0.05, *<0.05, **<0.005. P-values are computed using linear regression. c. ELISA-based epitope mapping of S2 stem-helix bnAbs with SARS-CoV-2 stem alanine scan peptides (25mer). Heatmap shows fold-changes in EC₅₀ binding titers of mAb binding to SARS-CoV-2 stem-helix peptide alanine mutants compared with the WT peptide. SARS-CoV-2 stem-helix residue positions targeted (2-fold or higher decrease in EC₅₀ binding titer compared to WT stem peptide) is indicated. Three hydrophobic residues, F¹¹⁴⁸, L¹¹⁵² and F¹¹⁵⁶, were commonly targeted by stem-helix bnAbs and that form the core of the bnAb epitope. Association of dependence on the stem bnAb core epitope residues with heavy (IGHV1-46 and IGHV3-23) and light (IGKV3-20 and IGLV1-51) chain genes usage and CDRL3 lengths is shown. d-g. A SARS-CoV-2 spike protein cartoon depicts the S2-stem epitope region at the base of the prefusion spike ectodomain (d). Sequence conservation of stem-helix hydrophobic core epitope residues (F¹¹⁴⁸, L¹¹⁵² and F¹¹⁵⁶) across β-coronavirus spikes (SEQ ID NOs:281-288, respectively) (PDB: 6XR8) (e). D¹¹⁴⁶ stem-helix residue is also indicated. Side (f) and top (g) views of spike stem-helix region highlight the core epitope residues. h.i. Interactions between SARS-CoV-2 S2 stem helix with (h) S2P6 and (i) CC40.8 highlighting the contribution of antibody germline-encoded residues in recognition of hydrophobic stem-helix core epitope. Structures with PDB codes 7RNJ and 7SJS are used for S2P6 and CC40.8, respectively.

FIG. 4. Prophylactic treatment of aged mice with S2 stem-helix bnAbs protects against challenge with diverse betacoronaviruses. a. Two S2 stem-helix bnAbs (CC68.109 and CC99.103) individually, or a DEN3 control antibody were administered intra-peritoneally (i.p.) at 300 μg per animal into 9 groups of aged mice (10 animals per group). Each group of animals was challenged intra-nasally (i.n.) 12 h after antibody infusion with one of 3 mouse-adapted (MA) betacoronaviruses, (MA10-SARS-2=SARS-CoV-2; 1×10³ plaque forming units (PFU), MA15-SARS1=SARS-CoV-1; 1×10⁵ PFU or M35c4-MERS=MERS-CoV; 1×10³ PFU). As a control, groups of mice were exposed only to PBS in the absence of virus. b., e., h. Percent weight change in S2 stem-helix bnAbs or DEN3 control antibody-treated animals after challenge with mouse-adapted betacoronaviruses. Percent weight change was calculated from day 0 starting weight for all animals. c., f., i. Day 2 post-infection Hemorrhage (Gross Pathology score) scored at tissue harvest in mice prophylactically treated with S2 stem-helix bnAbs or DEN3 control mAb. d., g., j. Day 2 post-infection pulmonary function (shown as Penh score) was measured by whole body plethysmography in mice prophylactically treated with S2 stem-helix bnAbs or DEN3 control mAb. Statistical comparisons between groups were performed using a Kruskal-Wallis non-parametric test and significance was calculated with Dunnett's multiple comparisons test between each experimental group and the DEN3 control Ab group. (p<0.05, **p<0.01, ***p<0.001; ****p<0.0001; ns-p>0.05).

FIG. 5. Flow cytometry B cell profiling, sorting strategy and SARS-CoV-2 and MERS-CoV S-protein specific B cells in infected-vaccinated donors. a. Gating strategy for analysis of IgG⁺ B cell populations that bind MERS-CoV S-protein only (CD3⁻CD4⁻CD8⁻CD14⁻CD19⁺CD20⁺IgM⁻IgG⁺CoV2⁻⁻MERS-CoV⁺), SARS-CoV-2 S-protein only (CD3⁻CD4⁻CD8⁻CD14⁻CD19⁺CD20⁺IgM⁻IgG⁺CoV2⁺⁺MERS-CoV⁻), or both MERS-CoV and SARS-CoV-2 S-proteins (CD3⁻CD4⁻CD8⁻CD14⁻CD19⁺CD20⁺IgM⁻IgG⁺CoV2⁺⁺MERS-CoV⁺). b. The frequencies of SARS-CoV-2 S-protein-specific IgG⁺ B cells (left), SARS-CoV-2 and MERS-CoV double positive S-protein-specific IgG⁺ cross-reactive B cells (middle) or MERS-CoV S-protein-specific IgG⁺ B cells (right) in PBMCs of 10 infected vaccined-vaccinated donors. c. Summary of the number of SARS-CoV-2 and MERS-CoV double positive S-protein specific cross-reactive B cells recovered from each of the donor, number and efficiency of heavy and light chain paired recovered, number of stem-helix mAb in each donor and their frequency out of the S-protein specific cross-reactive IgG B cells.

FIG. 6. Binding and immunogenetic properties of the isolated S2 stem-helix mAbs. A total of 40 S2 stem-helix mAbs from 9 SARS-CoV-2 infected-vaccinated donors (CC9 (n=10), CC24 (n=2), CC25 (n=7), CC67 (n=3), CC68 (n=2), CC84 (n=2), CC92 (n=2), CC95 (n=9) and CC99 (n=3) were isolated by single B cell sorting using SARS-CoV-2 and MERS-CoV S-proteins as baits. 32 out of 40 mAb were encoded by unique gene families. Heavy (V, D, J) and light (V, J) germline gene usage, CDR3 lengths and somatic hypermutation (SHM) levels are shown. MAbs were expressed and tested for binding to soluble (ELISA) and cell surface expressed (Cell-ELISA) spikes derived from human β-(SARS-CoV-1 or 2, MERS-CoV, HCoV-HKU1 and HCoV-OC43) and α-(HCoV-NL63 and HCoV-229E) coronaviruses and EC₅₀ and MFI (mean fluorescent intensity) binding values are shown. S2 stem-helix bnAbs show binding to β- but not α-HCoV spikes. Binding to cell surface expressed spikes was relatively better compared to soluble S-proteins. Polyreactive binding analysis of S2 stem-helix bnAbs to HEp2 cells and by ELISA for binding against polyspecific reagents (PSR) including Chinese hamster ovary cells solubilized membrane protein (CHO-SMP), insulin and single-strand DNA (ssDNA). More details are included in FIG. 8. S2 stem helix bnAbs, CC40.8, S2P6 and CV3-25 were used as control for binding assays.

FIG. 7. Immunoglobulin heavy and light chain gene usage and enrichment in isolated mAbs compared to a reference human germline database. Baseline germline frequencies of heavy chain genes (IGHV, IGHD and IGHJ genes) (a., b., c) and light chain genes (IGKV, IGLV, IGKJ and IGLJ genes) (d., e) are shown in grey, and S2 stem helix sarbecovirus bnAbs (SARS: orange) and sarbecovirus+MERS-CoV bnAb (SARS+MERS) are shown. Arrows indicate gene enrichments compared to human baseline germline frequencies.

FIG. 8. Evaluation of stem-helix bnAbs for polyreactivity and autoreactivity. a-b. Antibodies were tested for binding to immobilized HEp2 cells (a) and by ELISA for binding against polyspecific reagents (PSR) including Chinese hamster ovary cells solubilized membrane protein (CHO-SMP), insulin and single-strand DNA (ssDNA) (b). For HEp2 assay, immunofluorescence showed binding of antibodies to immobilized HEp2 cells was detected by FITC-labelled secondary antibody. Fluorescent intensity from strong to weak were labeled as “++”, “+” and “+/−” accordingly. “−” indicated little or no signal could be observed. Positive and negative controls for the HEp2 assay are provided by the manufacturer. In PSR ELISA, Bococizumab which is a humanized mAb targeting the LDL receptor-binding domain of PCSK9 and studied in phase I-III clinical studies (88), was used as a positive control. The curves indicate antibodies that can react with PSR, while curves are the antibodies with little or no binding to PSR. DEN3 mAb was used as a negative control.

FIG. 9. BLI Binding of S2 stem bnAbs and their iGLs with SARS-CoV-2 and MERS-CoV stem-helix peptides and S-proteins and association with SHMs and Neutralization. a. BioLayer Interferometry (BLI) binding kinetics of 16 S2 stem-helix bnAbs and their inferred germline (iGL) Ab versions with SARS-CoV-2 and MERS-CoV stem-helix peptides and S-proteins. Binding kinetics were obtained using the 1:1 binding kinetics fitting model on ForteBio Data Analysis software and maximum binding responses, dissociations constants (K_{D}) and on-rate (kₒₙ) and off-rate constants (k_{off}) for each antibody peptide interaction are shown. K_{D}, kₒₙ and k_{off} values were calculated only for antibody-antigen interactions where a maximum binding response of 0.2 nm was obtained. MAbs were also tested with SARS-CoV-2 and MERS-CoV S-proteins and the responses, apparent binding constants (K_{D}^{App}) and kₒₙ and k_{off} constants for each antibody-antigen interaction are indicated. The iGL Ab versions of stem-helix bnAbs showed reduced binding compared their mature versions. b. Correlations of stem-helix mAb binding (K_{D} (M) values) to SARS-CoV-2 and MERS-CoV peptides and virus neutralization with heavy (VH) chain and light (VL) chain SHM levels. c. Correlations of stem-helix mAb binding (K_{D} (M) values) to SARS-CoV-2 and MERS-CoV peptides with neutralization against their corresponding viruses. Correlations were determined by nonparametric Spearman correlation two-tailed test with 95% confidence interval. The Spearman correlation coefficient (r) and p-value are indicated. d. neutralization of S2 stem-helix bnAb iGLs with SARS-CoV-2 and MERS-CoV.

FIG. 10. Immunogenetics analysis of heavy and light chain sequences of 32 unique S2 stem-helix mAbs. The phylogenetic tree represents concatenated heavy and light chain amino acid sequences of 32 S2 stem-helix mAbs. mAbs IDs are shown on the right. Four columns on the right show the following characteristics of mAbs (from left to right): (1) the germline V gene of each heavy chain (IGHV1-46, IGHV3-23, others), (2) the presence of RG motif in the amino acid sequence of each CDRH3 (motif is present, or motif is missing), (3) the germline V gene of each light chain (IGKV3-20, IGLV1-51, others), (4) the presence of WD motif in the amino acid sequence of each CDRL3 (motif is present, or motif is missing).

FIG. 11. Epitope mapping of S2 stem-helix bnAbs with SARS-CoV-2 stem-helix peptide alanine scan mutants. Heatmap showing EC₅₀ ELISA binding titers of S2-stem helix bnAbs to 25mer SARS-CoV-2 stem-helix peptide and its alanine scan mutants. Three hydrophobic residues, F¹¹⁴⁸, L¹¹⁵² and F¹¹⁵⁶ were commonly targeted by stem-helix bnAbs. S2 stem-helix bnAbs are grouped based on their heavy chain gene usage (IGHV1-46, IGHV3-23 and others). The light chain germline genes (IGKV3-20, IGLV1-51 and other) and CDRL3 lengths are shown. S2P6, CC40.8 and CV3-25 S2 stem-helix mAbs were used as controls.

FIG. 12. Neutralization of replication competent betacoronaviruses by select S2-stem helix bnAbs. a. Neutralization of replication competent viruses encoding SARS-CoV-2 (SARS2-nLuc), and MERS-CoV (MERS-nLuc) by 2 select S2 stem-helix bnAbs, CC68.109, and CC99.103. MERS-GD27 antibody (89) was a positive control for the MERS-CoV neutralization assay. b-c. Comparison of IC₅₀ neutralization titers of S2 stem-helix bnAbs with replication-competent (b) and pseudoviruses (c) of SARS-CoV-2 and MERS-CoV.

### DETAILED DESCRIPTION

### I. Overview

Pan-betacoronavirus neutralizing antibodies may hold the key to developing broadly protective vaccines against coronaviruses that cause severe disease, for anticipating novel pandemic-causing viruses, and to respond more effectively to SARS-CoV-2 variants. The emergence of the Omicron variant of SARS-CoV-2 has illustrated the limitations of solely targeting the receptor binding domain (RBD) of the envelope Spike (S)-protein. The present invention is derived in part from studies undertaken by the inventors to isolate a large panel of broadly neutralizing antibodies (bnAbs) from SARS-CoV-2 recovered-vaccinated donors that target a conserved S2 region in the fusion machinery on betacoronavirus spikes. It was found that some of the bnAbs show broad in vivo protection against all three pathogenic betacoronaviruses, SARS-CoV-1, SARS-CoV-2 and MERS-CoV, that have spilled over into humans in the past 20 years to cause severe disease. These bnAbs provide new opportunities for antibody-based interventions and key insights for developing pan-betacoronavirus vaccines.

In accordance with these studies, the invention provides broadly neutralizing antibodies against betacoronaviruses. These antibodies target the conserved S2 stem-helix region in the fusion machinery on betacoronavirus spikes. The bnAb binding site is highly conserved in SARS-like viruses and potentially emerging betacoronaviruses with human pandemic potential. Thus, the S2-binding bnAbs of the invention are promising for diagnostics, antibody-based interventions, and for prophylactic vaccine strategies. In particular, they provide a choice of optimal reagents for antibody-based prophylaxis and therapy to respond to the viral threat. As the S2-binding bnAbs target sites conserved across coronavirus spike proteins, they are highly effective against the SARS-CoV-2 variants of concern (VOCs) including the omicron variant. They will especially facilitate vaccine design and antibody-based intervention strategies against pan-betacoronaviruses.

The invention can employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al, ed. (1989) Molecular Cloning A Laboratory Manual (2nd ed.; Cold Spring Harbor Laboratory Press); Sambrook et al, ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al, eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al, eds. (1995) Protein Engineering, A Practical Approach (IRL Press at Oxford Univ. Press, Oxford, Eng.). General principles of antibodies and antibody—hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York; Stites et al, eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al, eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al, (Elsevier, Amsterdam); Goldsby et al, eds. (2000) Kuby Immunology (4th ed.; W.H. Freeman & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall, 2003); Harlow and Lane (1988) Antibodies: A Laboratory Manual (Cold Spring Harbor Press); Dieffenbach and Dveksler (2003) PCR Primer (Cold Spring Harbor Press).

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “antibody” also synonymously called “immunoglobulin” (Ig), or “antigen-binding fragment” refers to polypeptide chain(s) which exhibit a strong monovalent, bivalent or polyvalent binding to a given antigen, epitope or epitopes. Unless otherwise noted, antibodies or antigen-binding fragments used in the invention can have sequences derived from any vertebrate species. They can be generated using any suitable technology, e.g., single B cell cloning, hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments and other designer antibodies that are described below or well known in the art (see, e.g., Serafini, J Nucl. Med. 34:533-6, 1993).

An “intact antibody” (or “full length antibody”) typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4}. Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

An antibody-based binding protein, as used herein, may represent any protein that contains at least one antibody-derived V_{H}, V_{L}, or C_{H} immunoglobulin domain in the context of other non-immunoglobulin, or non-antibody derived components. The antibody-based binding proteins of the invention include, but are not limited to (i) Fc-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

“Binding affinity” is generally expressed in terms of equilibrium association or dissociation constants (K_{A} or K_{D}, respectively), which are in turn reciprocal ratios of dissociation and association rate constants (k_{off} and kₒₙ, respectively). Thus, equivalent affinities may correspond to different rate constants, so long as the ratio of the rate constants remains the same. The binding affinity of an antibody is usually be expressed as the K_{D} of a monovalent fragment (e.g. a F_{ab} fragment) of the antibody, with K_{D} values in the single-digit nanomolar range or below (subnanomolar or picomolar) being considered as very high and of therapeutic and diagnostic relevance.

As used herein, the term “binding specificity” refers to the selective affinity of one molecule for another such as the binding of antibodies to antigens (or an epitope or antigenic determinant thereof), receptors to ligands, and enzymes to substrates. Thus, all monoclonal antibodies that bind to a particular antigenic determinant of an entity (e.g., a specific epitope of SARS-CoV-2 spike) are deemed to have the same binding specificity for that entity.

Betacoronaviruses (β-CoVs or Beta-CoVs) refer to one of four genera (Alpha-, Beta-, Gamma-, and Delta-) of coronaviruses. Member viruses are enveloped, positive-strand RNA viruses that infect mammals including humans. The natural reservoir for betacoronaviruses are bats and rodents. Rodents are the reservoir for the subgenus Embecovirus, while bats are the reservoir for the other subgenera. The betacoronaviruses of the greatest clinical importance concerning humans are OC43 and HKU1 (which can cause the common cold) of lineage A, SARS-CoV and SARS-CoV-2 (which causes the disease COVID-19) of lineage B, and MERS-CoV of lineage C. MERS-CoV is the first betacoronavirus belonging to lineage C that is known to infect humans.

A “conservative substitution” with respect to proteins or polypeptides refers to replacement of one amino acid with another amino acid having a side chain with similar chemical properties. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Methods of identifying nucleotide and amino acid conservative substitutions which do not eliminate protein activity are well-known in the art (see, e.g., Brummell et ah, Biochem. 32: 1180-1 187 (1993); Kobayashi et ah, Protein Eng. 12(10):879-884 (1999); and Burks et al, Proc. Natl. Acad. Sci. USA 94:412-417 (1997)).

The term “conservatively modified variant” applies to both amino acid and nucleic acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or chemical compounds), combining agents and cells or biological samples, or combining two populations of different cells. Contacting can occur in vitro, e.g., mixing an antibody and a biological sample, or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

A “humanized antibody” is an antibody or antibody fragment, antigen-binding fragment, or antibody-based binding protein comprising antibody V_{H} or V_{L} domains with a homology to human V_{H} or V_{L} antibody framework sequences having a T20 score of greater than 80, as defined by defined by Gao et al. (2013) BMC Biotechnol. 13, pp. 55.

There are seven coronaviruses that infect humans, namely 229E, OC43, NL63, HKU1, severe acute respiratory syndrome coronavirus (SARS-CoV), Middle East respiratory syndrome coronavirus (MERS-CoV) and the novel coronavirus SARS-CoV-2 (aka “2019-nCoV”). Unlike the highly pathogenic SARS-CoV, MERS-CoV, and SARS-CoV-2, the four so-called endemic (or “common”) coronaviruses generally cause mild upper-respiratory tract illness and contribute to 15%-30% of cases of common colds in human adults, although severe and life-threatening lower respiratory tract infections can sometimes occur in infants, elderly people, or immunocompromised patients.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, Madison, WI); or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

“Pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

As used herein, S2 stem helix refers to the epitope formed by residues encompassing 1140 to 1164 region in the S2 subunit of SARS-CoV-2, or the corresponding region or conserved structural motif in the spikes of other coronaviruses. In SARS-CoV-2, this peptide motif folds as an amphipathic a helix. In many prefusion cryo-EM structures of SARS-CoV-2 spike and other coronavirus spikes, the S2 stem helix forms a three-helix bundle. Detailed structure and sequence information of the S2 stem helix in various coronaviruses are described in, e.g., Pinto et al., Science 373, 1109-1116, 2021; and Zhou et al., Sci. Transl. Med. 10.1126/scitranslmed.abi9215, 2022.

The term “subject” refers to human and non-human animals (especially non-human mammals). The term “subject” is used herein, for example, in connection with therapeutic and diagnostic methods, to refer to human or animal subjects. Animal subjects include, but are not limited to, animal models, such as, mammalian models of conditions or disorders associated with coronavirus infections. Other specific examples of non-human subjects include, e.g., cows, horses, sheep, pigs, cats, dogs, mice, rats, rabbits, guinea pigs, monkeys.

The terms “treat,” “treating,” “treatment,” and “therapeutically effective” used herein do not necessarily imply 100% or complete treatment. Rather, there are varying degrees of treatment recognized by one of ordinary skill in the art as having a potential benefit or therapeutic effect. In this respect, the inventive method can provide any amount of any level of treatment. Furthermore, the treatment provided by the inventive method can include the treatment of one or more conditions or symptoms of the disease being treated.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

### III. Compositions Derived from nAbs Binding to Betacoronavirus S2 Stem Helix

The invention provides novel broadly neutralizing antibodies against betacoronaviruses (e.g., SARS-CoV-2), modified antibodies derived from the antibodies exemplified herein, as well as related fusion or conjugate molecules (e.g., antibody-drug conjugates), pharmaceutical compositions, other related compositions, and methods for producing the antibody compositions. As detailed herein, the antibody compositions are capable of neutralizing a betacoronavirus by specifically binding to a conserved epitope on the S2 subunit of the viral spike protein. In some embodiments, the antibodies or antigen-binding fragments thereof of the invention are capable of neutralizing one or more betacoronaviruses. In some embodiments, the antibodies or antigen-binding fragments thereof specifically recognizes the S2 stem helix epitope in the S2 subunit of betacoronaviruses. In some embodiments, the antibodies or antigen-binding fragments of the invention are derived from one of the exemplified antibodies described in the Examples below (e.g., Table 1). Typically, they have identical or substantially identical heavy chain and light chain CDR sequences as that of one of the exemplified antibodies. Defined alternatively, they have the same binding specificity as that of one of the exemplified antibodies. In some embodiments, the antibodies or antigen-binding fragments have heavy chain and light CDR sequences that are respectively identical to the heavy chain and light chain CDR sequences listed in Table 1. In some embodiments, the antibodies or antigen-binding fragments thereof contain one or more amino acid substitutions relative to the heavy chain sequence and the light chain sequence of the exemplified antibody. In some embodiments, the substitutions can be located either in the framework region, in the Fc domain, or in the CDRs of the antibodies exemplified herein.

In various embodiments, the antibody or antigen-binding fragment of the invention can contain heavy chain CDR sequences (HCDR1-3) as set forth respectively in (1) SEQ ID NOs:81-83, (2) SEQ ID NOs:86-88, (3) SEQ ID NOs:91-93, (4) SEQ ID NOs:96-98, (5) SEQ ID NOs:101-103, (6) SEQ ID NOs:106-108, (7) SEQ ID NOs:111-113, (8) SEQ ID NOs:116-118, (9) SEQ ID NOs:121-123, (10) SEQ ID NOs:126-128, (11) SEQ ID NOs:131-133, (12) SEQ ID NOs:136-138, (13) SEQ ID NOs:141-143, (14) SEQ ID NOs:146-148, (15) SEQ ID NOs:151-153, (16) SEQ ID NOs:156-158, (17) SEQ ID NOs:161-163, (18) SEQ ID NOs:166-168, (19) SEQ ID NOs:171-173, (20) SEQ ID NOs:176-178, (21) SEQ ID NOs:181-183, (22) SEQ ID NOs:186-188, (23) SEQ ID NOs:191-193, (24) SEQ ID NOs:196-198, (25) SEQ ID NOs:201-203, (26) SEQ ID NOs:206-208, (27) SEQ ID NOs:211-213, (28) SEQ ID NOs:216-218, (29) SEQ ID NOs:221-223, (30) SEQ ID NOs:226-228, (31) SEQ ID NOs:231-233, (32) SEQ ID NOs:236-238, (33) SEQ ID NOs:241-243, (34) SEQ ID NOs:246-248, (35) SEQ ID NOs:251-253, (36) SEQ ID NOs:256-258, (37) SEQ ID NOs:261-263, (38) SEQ ID NOs:266-268, (39) SEQ ID NOs:271-273, or SEQ ID NOs:276-278. Additionally or alternatively, these molecules can contain light chain CDR sequences (LCDR1-3) as set forth respectively in (1) SEQ ID NO:84, GAS, and SEQ ID NO:85, (2) SEQ ID NO:89, GAS, and SEQ ID NO:90, (3) SEQ ID NO:94, WAS, and SEQ ID NO:95, (4) SEQ ID NO:99, GAS, and SEQ ID NO:100, (5) SEQ ID NO:104, GAS, and SEQ ID NO:105, (6) SEQ ID NO:109, WAS, and SEQ ID NO:110, (7) SEQ ID NO:114, SSY, and SEQ ID NO:115, (8) SEQ ID NO:119, YAS, and SEQ ID NO:120, (9) SEQ ID NO:124, GAS, and SEQ ID NO:125, (10) SEQ ID NO:129, GAS, and SEQ ID NO:130, (11) SEQ ID NO:134, GAS, and SEQ ID NO:135, (12) SEQ ID NO:139, GAS, and SEQ ID NO:140, (13) SEQ ID NO:144, DAS, and SEQ ID NO:145, (14) SEQ ID NO:149, AVS, and SEQ ID NO:150, (15) SEQ ID NO:154, GVS, and SEQ ID NO:155, (16) SEQ ID NO:159, DNN, and SEQ ID NO:160, (17) SEQ ID NO:164, ENN, and SEQ ID NO:165, (18) SEQ ID NO:169, GAS, and SEQ ID NO:170, (19) SEQ ID NO:174, GAS, and SEQ ID NO:175, (20) SEQ ID NO:179, DAS, and SEQ ID NO:180, (21) SEQ ID NO:184, EAS, and SEQ ID NO:185, (22) SEQ ID NO:189, GAS, and SEQ ID NO:190, (23) SEQ ID NO:194, GAS, and SEQ ID NO:195, (24) SEQ ID NO:199, GAS, and SEQ ID NO:200, (25) SEQ ID NO:204, SNN, and SEQ ID NO:205, (26) SEQ ID NO:209, AAS, and SEQ ID NO:210, (27) SEQ ID NO:214, GPS, and SEQ ID NO:215, (28) SEQ ID NO:219, GPS, and SEQ ID NO:220, (29) SEQ ID NO:224, GAS, and SEQ ID NO:225, (30) SEQ ID NO:229, GAS, and SEQ ID NO:230, (31) SEQ ID NO:234, ENN, and SEQ ID NO:235, (32) SEQ ID NO:239, ENN, and SEQ ID NO:240, (33) SEQ ID NO:244, END, and SEQ ID NO:245, (34) SEQ ID NO:249, WAS, and SEQ ID NO:250, (35) SEQ ID NO:254, AVS, and SEQ ID NO:255, (36) SEQ ID NO:259, AAS, and SEQ ID NO:260, (37) SEQ ID NO:264, ENN, and SEQ ID NO:265, (38) SEQ ID NO:269, GAS, and SEQ ID NO:270, (39) SEQ ID NO:274, GAS, and SEQ ID NO:275, or (40) SEQ ID NO:279, GAS, and SEQ ID NO:280.

In some embodiments, the antibody or antigen-binding fragment of the invention contains heavy chain CDRs (HCDR1-3) and light chain CDRs (LCDR1-3) sequences set forth respectively in (1) SEQ ID NOs:81-84, GAS, and SEQ ID NO:85; (2) SEQ ID NOs:86-89, GAS, and SEQ ID NO:90; (3) SEQ ID NOs:91-94, WAS, and SEQ ID NO:95; (4) SEQ ID NOs:96-99, GAS, and SEQ ID NO:100; (5) SEQ ID NOs:101-104, GAS, and SEQ ID NO:105; (6) SEQ ID NOs:106-109, WAS, and SEQ ID NO:110; (7) SEQ ID NOs:111-114, SSY, and SEQ ID NO:115; (8) SEQ ID NOs:116-119, YAS, and SEQ ID NO:120; (9) SEQ ID NOs:121-124, GAS, and SEQ ID NO:125; (10) SEQ ID NOs:126-129, GAS, and SEQ ID NO:130; (11) SEQ ID NOs:131-134, GAS, and SEQ ID NO:135; (12) SEQ ID NOs:136-139, GAS, and SEQ ID NO:140; (13) SEQ ID NOs:141-144, DAS, and SEQ ID NO:145; (14) SEQ ID NOs:146-149, AVS, and SEQ ID NO:150; (15) SEQ ID NOs:151-154, GVS, and SEQ ID NO:155; (16) SEQ ID NOs:156-159, DNN, and SEQ ID NO:160; (17) SEQ ID NOs:161-164, ENN, and SEQ ID NO:165; (18) SEQ ID NOs:166-169, GAS, and SEQ ID NO:170; (19) SEQ ID NOs:171-174, GAS, and SEQ ID NO:175; (20) SEQ ID NOs:176-179, DAS, and SEQ ID NO:180; (21) SEQ ID NOs:181-184, EAS, and SEQ ID NO:185; (22) SEQ ID NOs:186-189, GAS, and SEQ ID NO:190; (23) SEQ ID NOs:191-194, GAS, and SEQ ID NO:195; (24) SEQ ID NOs:196-199, GAS, and SEQ ID NO:200; (25) SEQ ID NOs:201-204, SNN, and SEQ ID NO:205; (26) SEQ ID NOs:206-209, AAS, and SEQ ID NO:210; (27) SEQ ID NOs:211-214, GPS, and SEQ ID NO:215; (28) SEQ ID NOs:216-219, GPS, and SEQ ID NO:220; (29) SEQ ID NOs:221-224, GAS, and SEQ ID NO:225; (30) SEQ ID NOs:226-229, GAS, and SEQ ID NO:230; (31) SEQ ID NOs:231-234, ENN, and SEQ ID NO:235; (32) SEQ ID NOs:236-239, ENN, and SEQ ID NO:240; (33) SEQ ID NOs:241-244, END, and SEQ ID NO:245; (34) SEQ ID NOs:246-249, WAS, and SEQ ID NO:250; (35) SEQ ID NOs:251-254, AVS, and SEQ ID NO:255; (36) SEQ ID NOs:256-259, AAS, and SEQ ID NO:260; (37) SEQ ID NOs:261-264, ENN, and SEQ ID NO:265; (38) SEQ ID NOs:266-269, GAS, and SEQ ID NO:270; (39) SEQ ID NOs:271-274, GAS, and SEQ ID NO:275; or (40) SEQ ID NOs:276-279, GAS, and SEQ ID NO:280.

In some embodiments, in addition to the CDR sequences noted above, the antibody or antigen-binding fragment of the invention also contains a heavy chain variable region and/or a light chain variable region sequences that are substantially identical (e.g., at least 90%, at least 95%, at least 99%, or 100% identical), respectively, to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

Antibodies of the invention include intact antibodies (e.g., IgG1 antibodies exemplified herein), antibody fragments or antigen-binding fragments, antibody-based binding proteins, which contain the antigen-binding portions of an intact antibody that retain capacity to bind to S2 stem helix of betacoronaviruses (e.g., SARS-CoV-2). Examples of such antibody fragments include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} or V_{L} domain (see, e.g., Ward et al., Nature 341:544-546, 1989); and (vii) an isolated complementarity determining region (CDR) as a linear or cyclic peptide. Examples of antibody-based binding proteins are polypeptides in which the binding domains of the antibodies are combined with other polypeptides or polypeptide domains, e.g. alternative molecular scaffolds, Fc regions, other functional or binding domains of other polypeptides or antibodies resulting in molecules with additional binding properties, e.g. bi- or multispecific proteins or antibodies. Such polypeptides can create an arrangement of binding or functional domains normally not found in naturally occurring antibodies or antibody fragments.

Antibodies of the invention also encompass “antibody fragments” (also termed “antigen-binding fragments” herein) that contain portions of an intact IgG antibody (e.g., the variant regions) responsible for target antigen recognition and binding. One example of such antibody fragments is single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules. In some embodiments, the invention provides modified antibodies or antigen-binding fragments that are derived from one of the S2 stem helix binding antibodies exemplified herein. In some of these embodiments, the modified antibodies contain heavy chain CDRs and/or light chain CDRs that are identical to that of the exemplified antibody, and substitution of one or more amino acid residues in the framework regions (e.g., conservative substitutions). In some of these embodiments, the modified antibodies contain heavy chain CDRs and/or light chain CDRs that are identical to that of the exemplified antibody, and one or more substitutions with non-natural amino acid residues (e.g., substitutions in the Fc domain). In some other embodiments, the modified antibodies contain heavy chain CDRs and/or light chain CDRs that are embedded in a heterologous antibody scaffold, e.g., a modified antibody scaffold described herein or other heterologous antibody scaffold known in the art.

Antibodies of the present invention also encompass single domain antigen-binding units, which have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

In general, the antibodies or antigen-binding fragments of the invention can be generated in accordance with routinely practiced immunology methods. Some of such methods are exemplified herein in the Examples. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, New York, 1998; Kohler & Milstein, Nature 256:495-497, 1975; Kozbor et al., Immunology Today 4:72, 1983; and Cole et al., pp. 77-96 in Monoclonal Antibodies and Cancer Therapy, 1985. In some embodiments, antigen-binding fragments of the S2 stem helix binding antibodies of the invention can be produced by enzymatic or chemical modification of the intact antibodies, or synthesized de novo using recombinant DNA methodologies, or identified using phage display libraries. Methods for generating these antibody fragments are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nat. Struct. Biol. 11:500-515, 2002; Ghahroudi et al., FEBS Letters 414:521-526, 1997; and Bond et al., J. Mol. Biol. 332:643-55, 2003. Other types of antigen-binding fragments (e.g., Fab, F(ab′)₂ or Fd fragments) can also be readily produced with routinely practiced immunology methods.

In some embodiments, an antibody or antigen-binding fragment of the invention can be further conjugated to a second moiety, which includes, e.g., a polypeptide and a small organic molecule. In some embodiments, the second moiety is a synthetic molecule such as a marker or detectable moiety (or label). In some embodiments, the second moiety is a drug moiety. In some of these embodiments, the conjugated drug moiety is a compound known to effective or useful for countering betacoronaviral infections. Examples include Remdesivir, Hydroxychloroquine, Favipiravir and Pirfenidone for treating SARS-CoV-2 infections. Conjugation of a second moiety to the antibodies of the invention can be readily carried our via routinely practiced methods that are well known in the art. For example, recombinant engineering and incorporated selenocysteine (e.g., as described in U.S. Pat. No. 8,916,159) can be used to conjugate a synthetic molecule. Other methods of conjugation can include covalent coupling to native or engineered lysine side-chain amines or cysteine side-chain thiols. See, e.g., Wu et al., Nat. Biotechnol, 23: 1 137-1 146 (2005).

### IV. Polynucleotides, Vectors and Host Cells for Producing S2 Stem Helix Binding bnAbs

The invention provides substantially purified polynucleotides (DNA or RNA) that are identical or complementary to sequences encoding polypeptides containing the heavy chain and/or light chain sequences of antibodies or antigen-binding fragments described herein, including segments or domains of the antibodies. In some embodiments, the polynucleotides of the invention encode the heavy chain or light chain sequences of broadly neutralizing antibodies that are derived from one of the exemplified antibodies, e.g., SEQ ID NOs:1-80. In some embodiments, the polynucleotides of the invention are cDNAs. When expressed from appropriate expression vectors, polypeptides encoded by these polynucleotides are capable of exhibiting betacoronavirus broadly neutralizing capacity. Also provided in the invention are polynucleotides which encode at least one CDR region and usually all three CDR regions from the heavy or light chain of the antibodies described herein (e.g., CDRs shown in Table 1). Some other polynucleotides encode all or substantially all of the variable region sequence of the heavy chain and/or the light chain of the exemplified antibodies (e.g., SEQ ID NOs:1-80).

The polynucleotides of the invention can encode only the variable region sequences of the exemplified antibodies. They can also encode both a variable region and a constant region of the antibody. Some of polynucleotide sequences of the invention nucleic acids encode a mature heavy chain variable region sequence that is substantially identical (e.g., at least 80%, 90%, 95% or 99%) to the mature heavy chain variable region sequence shown in any one of SEQ ID NOs:1-40. Some other polynucleotide sequences encode a mature light chain variable region sequence that is substantially identical (e.g., at least 80%, 90%, 95% or 99%) to the mature light chain variable region sequence shown in any one of SEQ ID NOs:41-80. Some of the polynucleotide sequences encode a polypeptide that comprises variable regions of the heavy chain or the light chain of one of the exemplified antibodies. Some other polynucleotides encode two polypeptide segments that respectively are substantially identical to the variable regions of the heavy chain or the light chain of one of the exemplified antibodies.

The polynucleotide sequences can be produced by de novo solid-phase DNA synthesis or by PCR mutagenesis of an existing sequence (e.g., sequences as described in the Examples below) encoding an exemplified functional antibody. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

Also provided in the invention are expression vectors and host cells for producing the functional antibodies described herein. Specific examples of plasmid and transposon based vectors for expressing the antibodies are described in the Examples below. Various other expression vectors can also be employed to express the polynucleotides encoding the functional antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the antibody polynucleotides and polypeptides in mammalian (e.g., human) cells include pCEP4, pREP4, pThioHis A, B & C, pcDNA3.1/His, pEBVHis A, B & C (Invitrogen, San Diego, CA), MPSV vectors, and numerous other vectors known in the art for expressing other proteins. Other useful nonviral vectors include vectors that comprise expression cassettes that can be mobilized with Sleeping Beauty, PiggyBack and other transposon systems. Useful viral vectors include vectors based on lentiviruses or other retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a functional antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of a functional antibody chain or fragment. These elements typically include an ATG initiation codon and adjacent ribosome binding site (Kozak consensus sequence) or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted functional antibody sequences. More often, the inserted functional antibody sequences are linked to a signal sequences before inclusion in the vector. Vectors to be used to receive sequences encoding the functional antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human, and preferably of human IgG1 antibodies.

The host cells for harboring and expressing the functional antibody chains can be either prokaryotic or eukaryotic. In some preferred embodiments, mammalian host cells are used to express and to produce the antibody polypeptides of the present invention. For example, they can be either a hybridoma cell line expressing endogenous immunoglobulin genes or a mammalian cell line harboring an exogenous expression vector. These include any normal mortal or normal or abnormal immortal animal or human cell. In addition to the cell lines exemplified herein, a number of other suitable host cell lines capable of secreting intact immunoglobulins are also known in the art. These include, e.g., the CHO cell lines, various HEK 293 cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer, and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, EF1α and human UbC promoters exemplified herein, the metallothionein promoter, the constitutive adenovirus major late promoter, the dexamethasone-inducible MMTV promoter, the SV40 promoter, the MRP pol III promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transformation is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate for the cell type.

The invention further provides eukaryotic or non-eukaryotic cells (e.g., T lymphocytes) that have been recombinantly engineered to produce the antibodies, antibody-based binding proteins or antibody fragments thereof of the invention. The eukaryotic or non-eukaryotic cells can be used as an expression system to produce the antibody of the invention. In some embodiments, the invention provides coronavirus spike targeting immune cells that are engineered to recombinantly express a broadly neutralizing antibody of the invention. For example, the invention provides a T cell engineered to express an antibody of the invention (e.g., an scFv, scFv-Fc, or (scFv)2), which is linked to a synthetic molecule containing one or more of the following domains: a spacer or hinge region (e.g., a CD28 sequence or a IgG4 hinge-Fc sequence), a transmembrane region (e.g., a transmembrane canonical domain), and an intracellular T-cell receptor (TCR) signaling domain, thereby forming a chimeric antigen receptor (CAR) or T-body. Intracellular TCR signaling domains that can be included in a CAR (or T-body) include, but are not limited to, CD3ζ, FcR-γ, and Syk-PT signaling domains as well as the CD28, 4-1BB, and CD134 co-signaling domains. Methods for constructing T-cells expressing a CAR (or T-body) are known in the art. See, e.g., Marcu-Malina et al., Expert Opinion on Biological Therapy, Vol. 9, No. 5 (posted online on Apr. 16, 2009).

### V. Therapeutic and Diagnostic Applications

The broadly neutralizing antibodies or antigen-binding fragments thereof disclosed herein can be used in various therapeutic and diagnostic applications. For example, they can be used alone or in a combination therapy in the prophylactic or therapeutic treatment of coronavirus infections (e.g., SARS-CoV-2 infection). In some embodiments, the invention provides methods of using the broadly neutralizing antibodies or fragments thereof to treat patients having infection by one or more coronaviruses (e.g., SARS-CoV-2 and SARS-CoV) or patients having other diseases or conditions associated with coronavirus infections. In some embodiments, the antibodies or antigen-binding fragments of the invention can be used to prevent infections by one or more coronaviruses, or to reduce or manage coronavirus-induced symptoms in a subject infected with one or more coronaviruses. In some other embodiments, the invention provides diagnostic methods for detecting coronavirus related infections or the presence of coronavirus in biological samples obtained from human subjects.

Pharmaceutical compositions containing one or more of the broadly neutralizing antibodies or antigen-binding fragments described herein are encompassed by the invention. In some embodiments, the pharmaceutical compositions are employed in therapeutic methods for treating coronavirus infections. Typically, the subject or patient suitable for treatment is one who has been or is suspected of having been exposed to one or more betacoronaviruses (e.g., SARS-CoV-2 or SARS-CoV), is infected or suspected of being infected with one or more coronavirus, has a betacoronavirus related disease, has a symptom of a betacoronavirus related disease, or has a predisposition toward contracting a betacoronavirus related disease. For example, the subject to be treated can be one who has been diagnosed of SARS-CoV-2 infection and/or possess symptoms associated with infections by one or more betacoronaviruses. The broadly neutralizing antibody or antigen-binding fragment thereof for use in the methods of the invention can a human or humanized antibody containing the same CDR sequences as that of one of the S2 stem helix binding antibodies exemplified herein. In some embodiments, the broadly neutralizing antibody or antigen-binding fragment thereof contains a binding domain that binds to the same epitope as, or competitively inhibits binding of, one or more of the antibodies exemplified herein.

In addition to the antibodies, pharmaceutical compositions of the invention typically also contain a pharmaceutically acceptable carrier, which is a molecule or substance that is normally not co-present naturally in a subject (e.g., a human patient) with a betacoronaviral S2 stem helix binding antibody described herein. Pharmaceutically carriers enhance or stabilize the composition, or to facilitate preparation of the composition. They should also be both pharmaceutically and physiologically acceptable in the sense of being compatible with the other ingredients and not injurious to the subject. Pharmaceutically acceptable carriers include solvents, dispersion media, coatings, antibacterial and antifungal agents, isotonic and absorption delaying agents, and the like that are physiologically compatible. The pharmaceutically acceptable carrier employed should be suitable for various routes of administration. Additional guidance for selecting appropriate pharmaceutically acceptable carriers is provided in the art, e.g., Remington: The Science and Practice of Pharmacy, Mack Publishing Co., 20^{th} ed., 2000. Some of the pharmaceutical compositions of the invention are vaccines. For vaccine compositions, appropriate adjuvants can be additionally included. Examples of suitable adjuvants include, e.g., aluminum hydroxide, lecithin, Freund's adjuvant, MPL™ and IL-12.

Therapeutic methods of the invention typically involve administering to a subject in need of treatment a pharmaceutical composition that contains a therapeutically amount of a broadly neutralizing antibody or antigen-binding fragment described herein (e.g., an antibody shown in Table 1). A therapeutically effective amount refers to an amount sufficient to achieve a therapeutic benefit, e.g., to ameliorate symptoms associated with betacoronavirus infections. Suitable amount to be administered can be readily determined by one of ordinary skill in the art without undue experimentation given the invention. Factors influencing the mode of administration and the respective amount of a betacoronavirus neutralizing antibody or antigen-binding fragment thereof include, but are not limited to, the severity of the disease, the history of the disease, and the age, height, weight, health, and physical condition of the individual undergoing therapy. Similarly, the amount of a broadly neutralizing betacoronavirus immunotherapeutic to be administered will be dependent upon the mode of administration and whether the subject will undergo a single dose or multiple doses of this agent. In some embodiments, the therapeutic methods of the invention can be employed in combination with other regimen for treating or controlling betacoronavirus infections. These include, e.g., remdesivir, Bamlanivimab, Casirivimab and Imdevimab cocktail, hydroxychloroquine and chloroquine, interferon β-1a, Azithromycin, Tocilizumab and other IL-6 inhibitors, Interferon-γ, or intravenous fluids and balancing electrolytes.

Methods of preparing and administering a broadly neutralizing antibody or antigen-binding fragment thereof provided herein, to a subject in need thereof are well known to or can be readily determined by those skilled in the art. The route of administration of a broadly neutralizing betacoronavirus antibody or antigen-binding fragment thereof can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as suitable forms, another example of a form for administration would be a solution for injection, in particular for intravenous or intraarterial injection or drip. In some cases a suitable pharmaceutical composition can comprise a buffer (e.g. acetate, phosphate or citrate buffer), a surfactant (e.g. polysorbate), optionally a stabilizer agent (e.g. human albumin), etc. In other methods compatible with the teachings herein, a broadly neutralizing antibody or antigen-binding fragment thereof as provided herein can be delivered directly to a site where the binding molecule can be effective in virus neutralization, e.g., the endosomal region of a coronavirus-infected cell. Preparation of pharmaceutical compositions of the invention and their various routes of administration can be carried out in accordance with methods well known in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978.

The invention also provided methods for using the broadly neutralizing antibodies or related antigen-binding fragments described herein in diagnostic methods for detecting betacoronavirus infections or the presence of betacoronaviruses. Various assays routinely practiced in the art can be employed for performing the diagnostic methods. These include, e.g., competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety). Methods and reagents suitable for determination of binding characteristics of a broadly neutralizing betacoronavirus antibody or antigen-binding fragment thereof are known in the art and/or are commercially available. Equipment and software designed for such kinetic analyses are commercially available (e.g., BIAcore®, BIAevaluation® software, GE Healthcare; KINEXA® Software, Sapidyne Instruments).

The diagnostic methods of the invention typically involve obtaining a biological sample from a subject that has or is suspected of having been infected with a coronavirus spike. Preferably, the subject is a human. In various embodiments, the biological sample suitable for the assays can be blood or any fraction thereof (e.g., serum, plasma, or whole blood), urine, feces, saliva, vomitus, or any combination thereof. Utilizing the novel antibodies disclosed herein, presence of a coronavirus spike or spike derived antigen in the biological sample can be readily determined with any of the various immunoassays described herein, e.g., ELISA.

The invention further provides kits that contain a broadly neutralizing betacoronavirus immunotherapeutic of the invention for performing the therapeutic or diagnostic applications described herein. Typically, the kits contain two or more components required for performing the therapeutic or diagnostic methods of the invention. Kit components include, but are not limited to, one or more the disclosed antibodies or antibody fragments thereof, appropriate reagents, and/or equipment. In some embodiments, the kits can contain an antibody or antibody fragment thereof of the invention and an immunoassay buffer suitable for detecting betacoronavirus spike proteins (e.g. by ELISA, flow cytometry, magnetic sorting, or FACS). The kit may also contain one or more microtiter plates, standards, assay diluents, wash buffers, adhesive plate covers, magnetic beads, magnets, and/or instructions for carrying out a method of the invention using the kit. The kits can include an antibody or antigen-binding fragment thereof of the invention bound to a substrate (e.g., a multi-well plate or a chip), which is suitably packaged and useful to detect betacoronavirus spike antigens. In some embodiments, the kits include an antibody or antibody fragment thereof of the invention that is conjugated to a label, such as, a fluorescent label, a biologically active enzyme label, a luminescent label, or a chromophore label. The kits can further include reagents for visualizing the conjugated antibody or antibody fragment thereof, e.g., a substrate for the enzyme. In some embodiments, the kits include an antibody or antibody fragment thereof of the invention that is conjugated to a contrast agent and, optionally, one or more reagents or pieces of equipment useful for imaging the antibody in a subject.

Generally, the broadly neutralizing antibodies or antibody fragments thereof of the invention in a kit are suitably packaged, e.g., in a vial, pouch, ampoule, and/or any container appropriate for a therapeutic or detection method. Kit components can be provided as concentrates (including lyophilized compositions), which may be further diluted prior to use, or they can be provided at the concentration of use. For use of the antibody of the invention in vivo, single dosages may be provided in sterilized containers having the desired amount and concentration of components.

In various applications, the broadly neutralizing antibodies of the invention can be employed to produce antibody derivatives such as immunoconjugates. In some embodiments, the antibodies of the invention can be linked to a therapeutic moiety, such as a cytotoxin, a drug or a radioisotope. When conjugated to a cytotoxin, these antibody conjugates are referred to as “immunotoxins.” A cytotoxin or cytotoxic agent includes any agent that is detrimental to (e.g., kills) cells. Techniques for conjugating such therapeutic moiety to antibodies are well known in the art. In some embodiments, antibodies of the invention can be conjugated to an appropriate detectable agent to form immunoconjugates for use in diagnostic applications and in vivo imaging. The detectable agents can be any chemical moieties that contain a detectable label, e.g., radioisotopes, enzymes, fluorescent labels and various other antibody tags. In some other embodiments, the broadly neutralizing antibodies of the invention can be further modified to contain additional non-proteinaceous moieties that are known in the art and readily available. The moieties suitable for derivatization of the antibody include but are not limited to water soluble polymers, e.g., polyethylene glycol (PEG).

### EXAMPLES

The following examples are provided to further illustrate the invention but not to limit its scope. Other variants of the invention will be readily apparent to one of ordinary skill in the art and are encompassed by the appended claims.

### Example 1. Donors for Isolation of β-CoV Spike Stem-Helix bnAbs

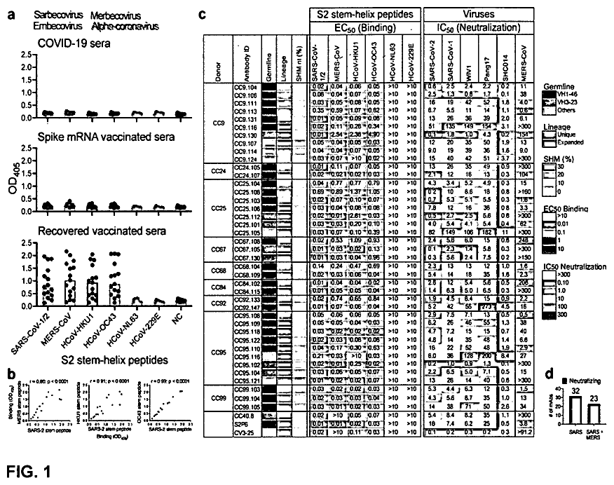

To identify suitable donors for the isolation of a panel of β-CoV spike stem-helix bnAbs, we screened immune sera from human donors for cross-reactive binding to 25-mer spike stem-helix region peptides, which we previously identified as a target for bnAbs (43, 44). We tested sera from three different groups of donors: i) COVID-19 recovered donors (n=15); ii) spike mRNA-vaccinated (2×) donors (n=10) and iii) COVID-19-recovered then spike-vaccinated (1×) donors (n=15) (FIG. 1-a). Whereas weak or no binding was observed for COVID-19 recovered or vaccinee sera to human β-CoV spike stem-helix peptides, sera from 80% (12/15) of recovered-vaccinated donors exhibited strong cross-reactive binding to the peptides (FIG. 1-a). We noted a strong correlation between binding of recovered-vaccinated sera to SARS-CoV-2 stem-helix peptide with binding to other human β-CoV stem-helix peptides suggesting targeting of common cross-reactive epitopes (FIG. 1-b). Accordingly, we sought to isolate β-CoV stem-helix directed bnAbs from 10 SARS-CoV-2 recovered-vaccinated donors that exhibited cross-reactive binding to this spike region.

### Example 2. Isolation of a Large Panel of β-CoV Spike Stem-Helix mAbs

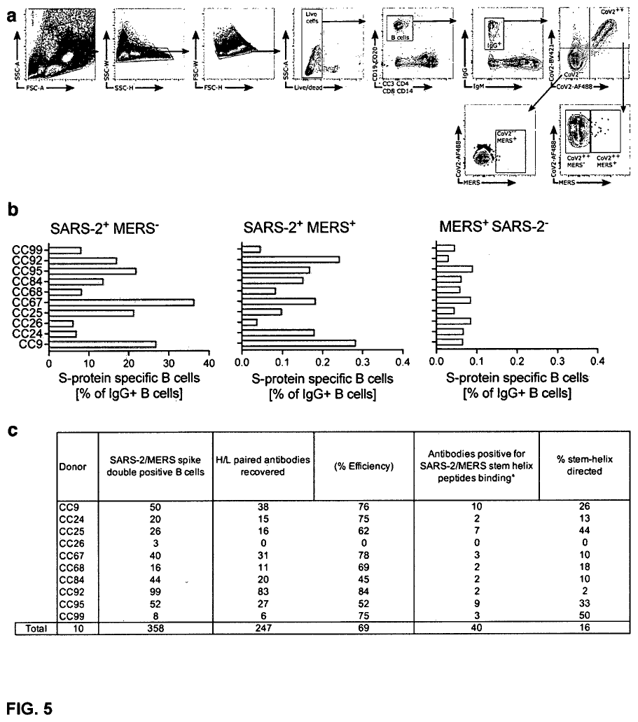

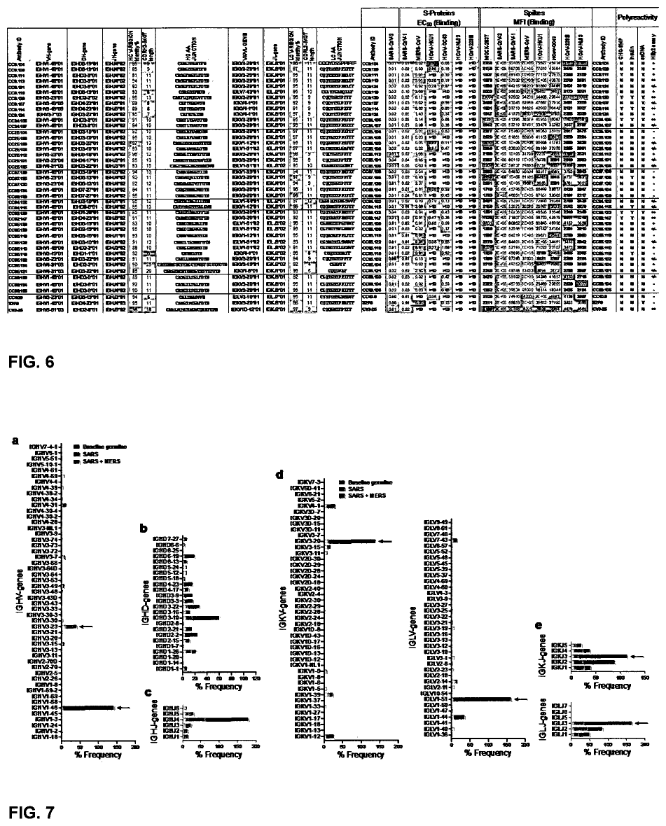

Using SARS-CoV-2 and MERS-CoV S-proteins as baits, we sorted antigen-specific single B cells to isolate 40 stem-helix mAbs from 10 COVID-19 convalescent donors who had been recently vaccinated with the Pfizer/BioNTech BNT162b2 (n=4: CC9, CC92, CC95 and CC99), Johnson & Johnson Ad26.CoV2.S (n=1: CC67), or Moderna mRNA-1273 (n=5: CC24, CC25, CC26, CC67, CC84) vaccines (FIG. 1-c and d, and FIG. 5) (2, 58, 59). Briefly, using SARS-CoV-2 and MERS-CoV S-proteins, we sorted CD19⁺CD20⁺IgG⁺IgM⁻ B cells positive for both probes from the peripheral blood mononuclear cells (PBMCs) of these donors. Flow cytometry profiling revealed up to 36% (range=6-36%, median=15%) SARS-CoV-2 S-protein-specific B cells, of which a sizable fraction was cross-reactive with the MERS-CoV S-protein (range=0.04-0.28%, median=0.16% total selected B cells) (FIG. 5-b). A total of 358 SARS-CoV-2: MERS-CoV S-protein-specific double positive single B cells were recovered from the 10 donors, of which the heavy (HC)-light (LC) chain pairs were recovered from 247 single B cells (69%) from 9 donors and expressed as IgGs (FIG. 5-c). Expi293F cell-expressed IgG supernatants of 247 mAbs were screened for dual binding to SARS-CoV-2 and MERS-CoV stem-helix peptides and 16% (40/247) exhibited cross-reactive binding (FIG. 5-c). Dual binding was confirmed for the corresponding purified IgGs. Except for two mAbs that failed to bind HCoV-HKU1 stem-helix peptide, all mAbs exhibited cross-reactive binding to stem-helix peptides of endemic β-HCoV (HCoV-HKU1 and HCoV-OC43) but not α-HCoV (HCoV-NL63 and HCoV-229E) (FIG. 1-c). We also tested binding of mAbs to soluble HCoV S-proteins and cell surface expressed spikes and observed consistent binding to SARS-CoV-2/1 and MERS-CoV spikes but reduced binding to endemic β-HCoV spikes (HCoV-HKU1 and HCoV-OC43), especially in the soluble S-protein format (FIG. 6). Overall, we isolated 40 stem-helix mAbs, of which 32 were encoded by unique immunoglobulin germline gene combinations and 7 were expanded lineages with 2 or more clonal members (FIG. 1-c, and FIG. 6).

### Example 3. Spike Stem-Helix mAbs Exhibit Broad Neutralization Against β-CoVs

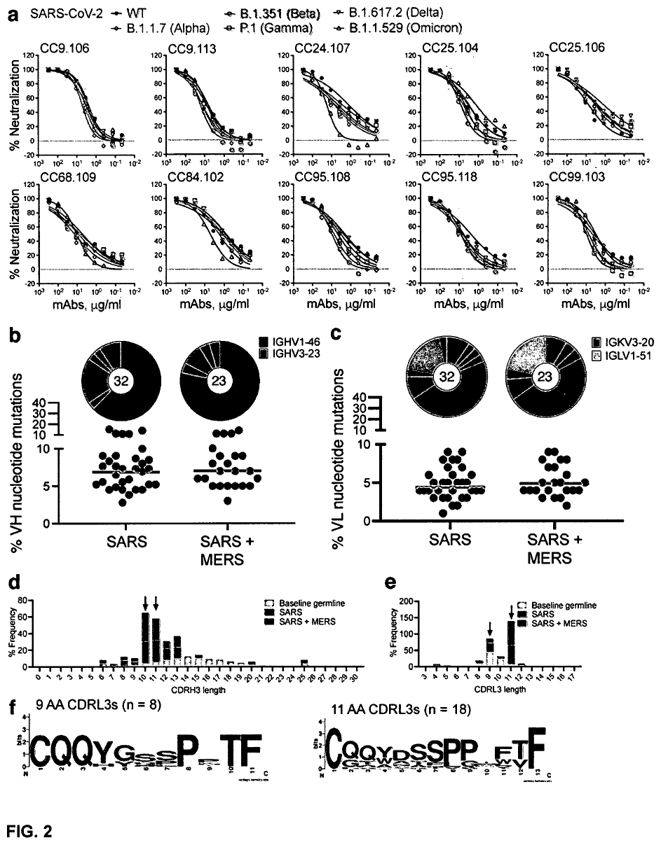

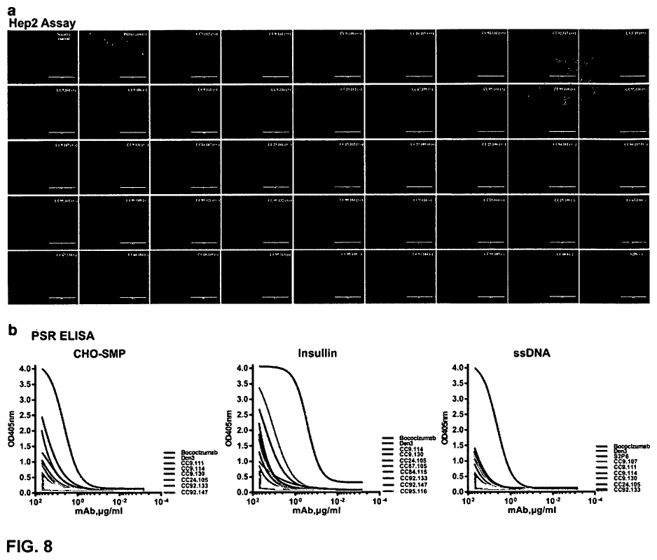

We next examined neutralization of stem-helix mAbs against clade 1a (SARS-CoV-1, WIV1 and SHC014) and clade 1b (SARS-CoV-2 and Pang17) ACE2-utilizing sarbecoviruses (26, 27) and MERS-CoV (28). Consistent with conservation of the stem-helix bnAb epitope region across sarbecoviruses, all the 32 mAb lineages neutralized all the 5 sarbecoviruses tested with widely varying degrees of neutralization potency (FIG. 1-c and d). The bnAbs neutralized clade 1a SHC014 and clade 1b SARS-CoV-2 relatively more potently compared to the other sarbecoviruses, but some bnAbs neutralized all viruses in the lower μg/mL neutralization titer range (0.1 to 3 μg/ml). Of 32 unique stem-helix bnAb lineages, 23 (72%) bnAbs neutralized MERS-CoV (FIG. 1-c and d). Neutralization potency against MERS-CoV was lower compared to the sarbecoviruses but many bnAb members were consistently effective. We tested neutralization of SARS-CoV-2 VOCs (B.1.1.7 (Alpha), B.1.351 (Beta), P.1 (Gamma), B.1.617.2 (Delta) and B.1.1.529 (Omicron) by select bnAbs (FIG. 2-a). Consistent with the conservation of the stem-helix region in SARS-CoV-2 VOCs, these bnAbs were consistently effective against the VOCs tested (FIG. 2-a). Of note, a fraction of stem-helix bnAbs showed some degree of polyreactivity or autoreactivity in HEp2 cell or polyspecificity reagent (PSR) assays (7) but the majority were negative (FIGS. 6 and 8). Overall, we have identified multiple stem-helix bnAbs that exhibit broad neutralizing activity against phylogenetically diverse β-HCoVs.

### Example 4. Immunogenetics of Stem-Helix bnAbs and Vaccine Targeting

Immunogenetic analysis of stem-helix antibody sequences showed strong enrichment of IGHV1-46 (63%) and IGHV3-23 (22%) germline gene families as compared to human baseline germline frequencies (FIG. 1-c, FIG. 2-b, and FIG. 7) (60, 61). Of note, previously isolated stem-helix human bnAbs, S2P6 and CC40.8, are IGHV1-46 and IGHV3-23 germline encoded, respectively (44, 45). The IGHV1-46 germline gene was slightly more enriched (78%) in stem-helix bnAbs that exhibited MERS-CoV neutralization in addition to sarbecoviruses, suggesting a potential role for this VH-germline gene for broader reactivity against diverse β-HCoV spikes. Interestingly, at least one IGHV1-46-encoded stem-helix bnAb was isolated from each of the 9 donors and may represent a public clonotype for this bnAb site. For light chain gene usage, we noted a strong enrichment of IGKV3-20 (47%) and to some degree IGLV1-51 (16%) germline gene families as compared to human baseline germline frequencies (FIG. 1-c, FIG. 2-c, and FIG. 7) (62). The mAbs possessed modest levels of V-gene nucleotide somatic hypermutation (SHM): for V_{H}, median=7.3% and for VL, median=4.5% (FIG. 6).

We examined the CDRH3 loop lengths in the isolated stem-helix bnAbs and observed a strong enrichment for 10- and 11-residue long CDRH3 s compared to the human baseline reference database (FIG. 2-d, and FIG. 7) (60, 61). No apparent enrichment in germline D-genes was observed but IGHJ4, the most common germline J-gene utilized in humans, was slightly enriched (72%) in stem-helix bnAbs compared to a reference germline database (FIG. 7) (60, 61). We also examined the CDRL3 loop lengths in the stem-helix bnAbs and observed strong enrichment for 9- and 11-residue CDRL3 s (FIG. 2-e, and FIGS. 6 and 7). These CDRL3 loops possess germline JL-gene-encoded motifs (FIG. 2-f, and FIG. 6), which may be important for epitope recognition. Overall, we observed a strong enrichment of IGHV and IGLV germline gene features in β-HCoV spike stem-helix bnAbs. Therefore, rational vaccine strategies may exploit these germline gene features to generate a protective B cell response (53, 54, 63).

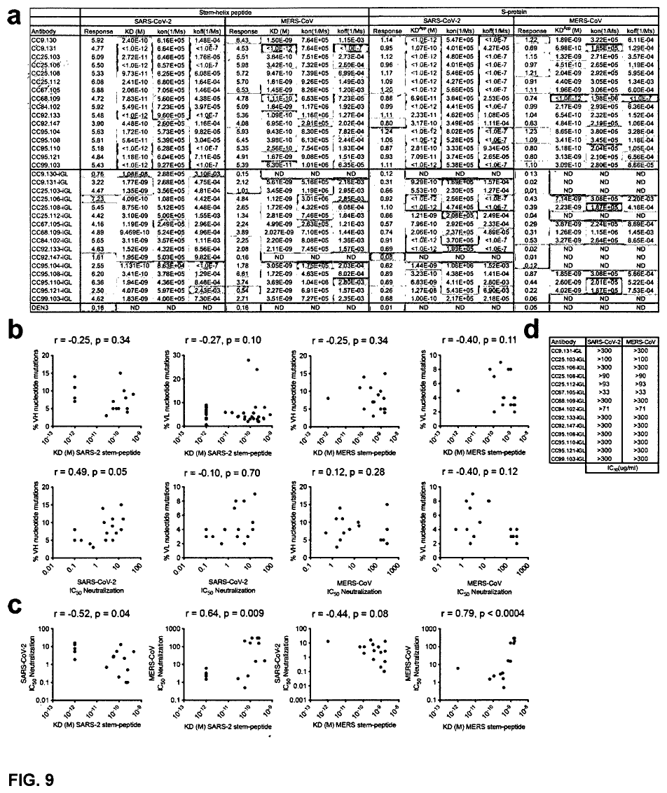

To examine the potential contribution of antibody SHMs to SARS-CoV-2 neutralization efficiency and cross-neutralization with MERS-CoV, we tested the binding of select mAbs (based on a broad range of neutralization potency) to SARS-CoV-2 or MERS-CoV monomeric stem-helix peptides and to their S-proteins by BLI (FIG. 9). The mAbs bind SARS-CoV-2 and MERS-CoV-2 stem-helix peptides with nanomolar (nM) and higher K_{D} affinity (FIG. 9-a) and were generally higher for SARS-CoV-2 compared to MERS-CoV stem-helix peptide. We found no association of heavy or light chain SHMs with binding to SARS-CoV-2 or MERS-CoV-2 stem-helix peptides or with neutralization of the corresponding viruses (FIG. 9-b). We however observed a strong association of binding affinity to stem-helix peptides and neutralization (FIG. 9-c).

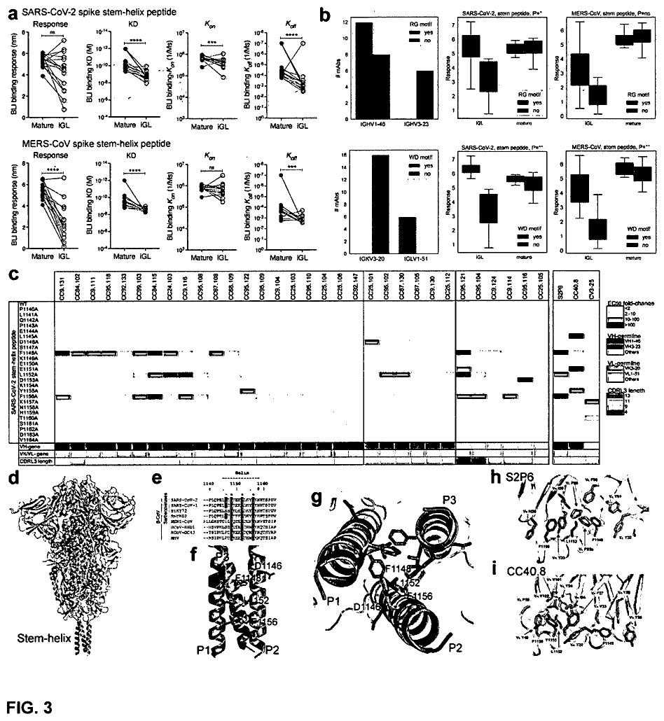

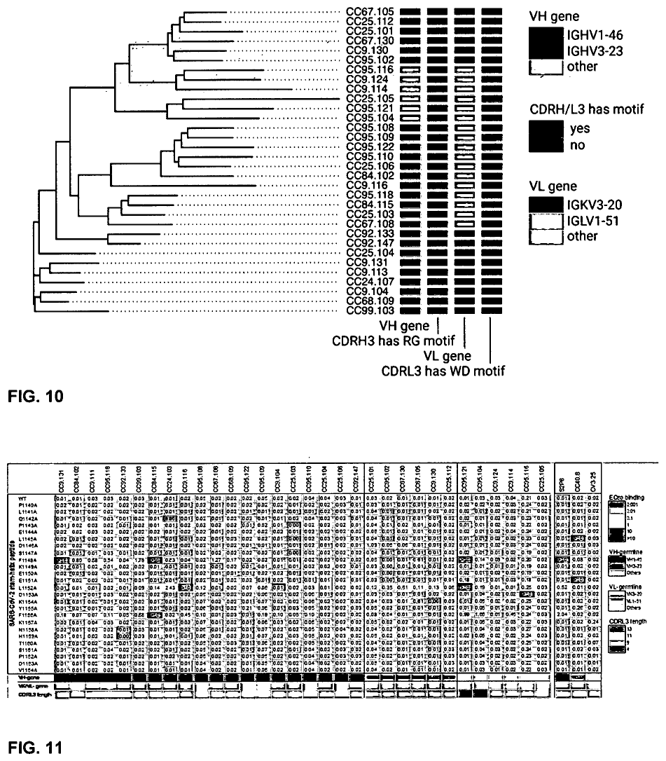

To further investigate the role of SHM in binding and neutralization, we generated inferred germline (iGL) versions of stem-nAbs by reverting their heavy and light chain V, D and J regions to the corresponding germlines (inferred germlines, iGLs) as described previously (64) and assessed both binding and neutralization. The BLI binding responses and the K_{D} values of the bnAb iGLs with SARS-CoV-2 and MERS-CoV stem-helix peptides were substantially reduced compared to mature bnAbs but were still strong and in the lower nM and higher K_{D} affinity range (FIG. 3-a, and FIG. 9-a). We observed higher affinities or CDRH3 “RG” motif-bearing IGHV1-46-encoded and CDRL3 “WD” motif-bearing IGLV1-51-encoded bnAb iGLs for binding to SARS-CoV-2 or MERS-CoV stem-helix peptides (FIG. 3-a, and FIG. 10). Binding of bnAbs and their iGLs to S-proteins were generally of higher affinity than to the corresponding peptides, possibly due to avidity effects (FIG. 9-a). The affinities of iGLs compared to mature bnAbs were notably less for S-proteins compared to the corresponding peptides, particularly for the MERS-CoV S-protein where many of the iGL Abs failed to bind substantially (FIG. 9-a). Overall, these results suggest a significant contribution from germline-encoded residues to epitope binding, in most cases consistent with enrichment of certain antibody germline gene features above (FIGS. 1 and 2).

In contrast to binding, neutralization of SARS-CoV-2 and MERS-CoV by stem-helix bnAb iGLs was absent (FIG. 9-d). The result suggests that, although overall SHM levels do not correlate with binding or neutralization, key antibody mutations are critical for the neutralization phenotype to attain sufficient affinity for neutralization to be observed.

Altogether, we have isolated a large panel of human β-CoV bnAbs that are enriched in certain germline gene features suggesting the potential value of a highly targeted approach (53, 54, 63) to induce pan-betacoronavirus bnAbs by vaccines in which the immunogen and vaccination strategies are appropriately designed.

### Example 5. Spike Stem-Helix bnAbs Recognize a Common Hydrophobic Core Epitope

To determine the epitope specificities of the isolated stem-helix bnAbs and potential association with antibody immunogenetic properties, we performed binding of all 32 stem bnAbs to alanine scanning mutants of the SARS-CoV-2 stem-peptide (FIG. 3-c, and FIG. 11). A dependence on three hydrophobic residues, F¹¹⁴⁸, L¹¹⁵², and F¹¹⁵⁶, by many bnAbs that form a common core epitope was identified but the relative dependence of bnAb lineages on each of the hydrophobic core residues varied. Many of the IGHV1-46-encoded bnAbs were paired with IGVK3-20 or IGLV1-51 light chain and all except two bnAbs possessed a CDRL3 of 11 residues. The IGHV3-23-encoded bnAbs showed dependence on 1 or 2 hydrophobic core epitope residue and some lineages showed dependence on an upstream acidic residue, D¹¹⁴⁶. All of the IGHV3-23 encoded bnAbs were paired with a IGVK3-20 light chain with a 9-residue long CDRL3 loop. The non-IGHV1-46 or -IGHV3-23-encoded stem-helix bnAbs were also dependent on one or more hydrophobic core epitope residues with one exception. Structural analysis of the IGHV1-46-encoded S2P6 or IGHV3-23-encoded CC40.8 stem-helix bnAbs shows that antibody germline gene-encoded residues are involved in recognition of the hydrophobic bnAb epitope (FIG. 3-d to i). Overall, hydrophobic core residues in the spike fusion machinery, which are highly conserved across betacoronaviruses, are important targets for S2 bnAbs. Notably, the hydrophobic core epitope residues on the pre-fusion S-trimer are poorly accessible and partial disruption of the stem-helix region may be needed to favorably expose this bnAb site to engage desired B cell responses (43-45, 48).

### Example 6. Stem-Helix bnAbs Protect Against Challenge with Diverse β-CoVs

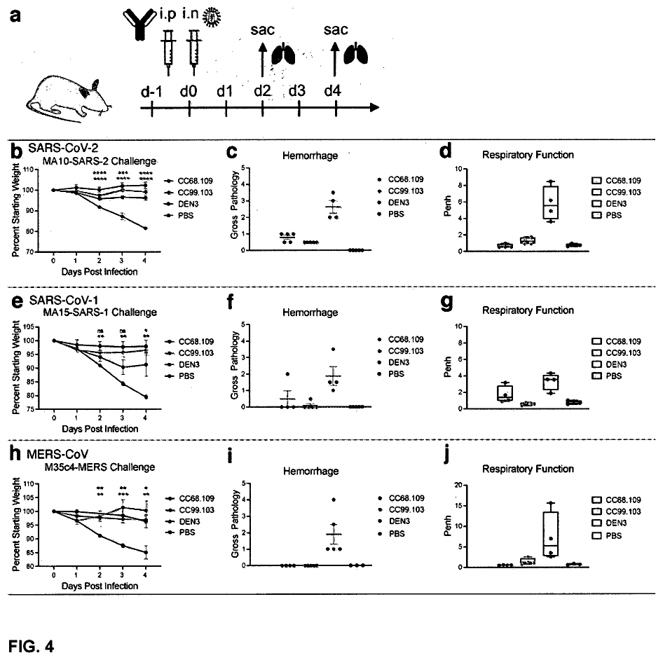

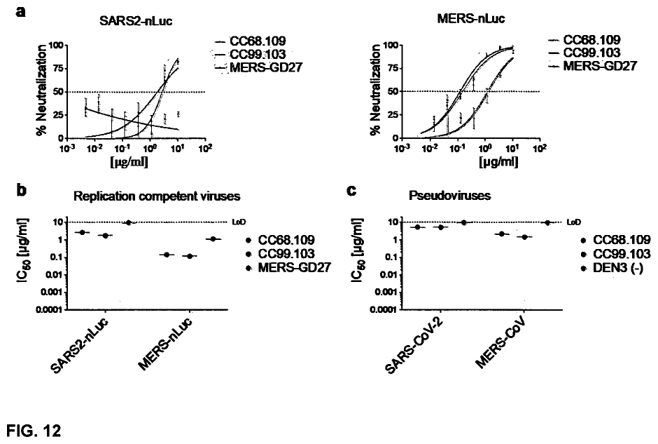

To determine the protective efficacy of the stem-helix bnAbs, we prophylactically treated aged mice (65) with individual antibodies followed by virus challenge. We selected two of the broadest and potent stem-helix bnAbs, CC68.109 and CC99.103, and investigated their in vivo protective efficacy against all three major human disease-causing betacoronaviruses; SARS-CoV-2, SARS-CoV-1 and MERS-CoV. Prior to the challenge experiments, we examined neutralization of SARS-CoV-2 and MERS-CoV replication-competent viruses by the two candidate bnAbs and compared with that of pseudoviruses (FIG. 12). The neutralization IC₅₀ s of the stem-helix bnAbs were comparable for SARS-CoV-2 across the two assay formats while the titers with replication-competent MERS-CoV were more effective (lower IC₅₀ values) compared to the pseudovirus format. The two stem-helix bnAbs, individually, or a DEN3 control antibody were administered intra-peritoneally (i.p.) at 300 μg/animal into 9 groups of 10 animals (3 groups per antibody; FIG. 4-a). 12 h prior to the virus challenge, the test antibody in each animal group was administered followed by intranasal (i.n.) challenge with one of three mouse-adapted (MA) betacoronaviruses, (MA10-SARS-2=SARS-CoV-2; MA15-SARS-1=SARS-CoV-1 or M35c4-MERS=MERS-CoV) (FIG. 4-a) (65-67). Post virus challenge, the animals were monitored for signs of clinical disease due to infection, including daily weight changes, and pulmonary function. Animals were euthanized at day 2 or day 4 post infection and lung tissues were harvested to assess gross pathology. Compared to the control antibody DEN3-treated animal groups, the stem-helix bnAb-treated animals in all three betacoronaviruses challenge experiments showed substantially reduced weight loss (FIG. 4b, e, h), reduced hemorrhage (FIG. 4-c, f and i), and normal pulmonary function (FIG. 4-d, g and j), suggesting a protective role for the bnAbs.

Overall, both stem-helix bnAbs protected against severe betacoronavirus disease, CC99.103 being slightly more protective than CC68.109 bnAb.

### Example 6. Materials and Methods

COVID-19 infected-vaccinated donors: Sera and PBMC samples from convalescent COVID-19 donors, vaccinated donors, and COVID-19-recovered vaccinated donors, were provided through the “Collection of Biospecimens from Persons Under Investigation for 2019-Novel Coronavirus Infection to Understand Viral Shedding and Immune Response Study” UCSD IRB #200236 as reported earlier (35). The protocol was approved by the UCSD Human Research Protection Program. Convalescent donor samples were collected based on COVID-19 diagnosis regardless of gender, race, ethnicity, disease severity, or other medical conditions. All human donors were assessed for medical decision-making capacity using a standardized, approved assessment, and voluntarily gave informed consent prior to being enrolled in the study.

Plasmid construction: To generate soluble S ectodomain proteins from SARS-CoV-1 (residues 1-1190; GenBank: AAP13567), SARS-CoV-2 (residues 1-1208; GenBank: MN908947), HCoV-HKU1 (residue 1-1295; GenBank: YP_173238.1), HCoV-OC43 (residues 1-1300; GenBank: AAX84792.1), MERS-CoV (residues 1-1291; GenBank: APB87319.1), HCoV-229E (residues 1-1110; GenBank: NP_073551.1) and HCoV-NL63 (residues 1-1291; GenBank: YP_003767.1), we synthesized the DNA fragments from GeneArt (Life Technologies) and cloned them into the phCMV3 vector (Genlantis cat. #P003300). In order to produce the stable trimeric prefusion spike proteins, double proline substitutions (2P) were introduced into the S2 subunit: K968/V969 in SARS-CoV-1, K986/V987 in SARS-CoV-2, V1060/L1061 in MERS-CoV, A1071/L1072 in HCoV-HKU1, A1078/L1079 in HCoV-OC43, S1052/I1053 in HCoV-NL63 and T871/I872 in HCoV-229E were replaced by proline. The furin cleavage sites (in SARS-CoV-2 residues 682-685, in SARS-CoV-1 residues 664-667, in HCoV-HKU1 residues 756-760, in HCoV-OC43 residues 762-766, in MERS-CoV residues 748-751, in HCoV-229E residues 564-567 and in HCoV-NL63 residues 745-748) were replaced by a “GSAS” linker; the trimerization T4 fibritin motif was incorporated at the C-terminus of the S proteins. To purify and biotinylate the spike proteins, the HRV-3C protease cleavage site, 6×HisTag, and AviTag spaced by GS-linkers were added to the C-terminus after the trimerization motif. To generate pseudoviruses of MERS-CoV and sarbecoviruses, the DNA fragments encoding the spikes of MERS-CoV and sarbecoviruses without the ER retrieval signal were codon-optimized and synthesized at GeneArt (Life Technologies). The spike encoding genes of Pang17 (residues 1-1249, GenBank: QIA48632.1), WIV1 (residues 1-1238, GenBank: KF367457) and SHC014 (residue 1-1238, GenBank: AGZ48806.1) were constructed into the phCMV3 vector (Genlantis cat. #P003300) using the Gibson assembly (New England Biolabs, cat. #E2621L) according to the manufacturer's instructions.

Cell lines: FreeStyle293-F cells (Thermo Fisher Scientific cat. #R79007) were grown in FreeStyl 293 Expression Medium (Gibco cat. #12338018), and Expi293F cells (Gibco cat. #A14527) were maintained in Expi293 Expression Medium (Gibco cat. #A1435101). Suspension cells were incubated in the shaker at 150 rpm, 37° C., 8% CO₂. Adherent HEK293T cells and HeLa-ACE2 cells were grown in Dulbecco's Modified Eagle Medium (DMEM) with 10% heat-inactivated FBS, 4 mM L-Glutamine and 1% P/S, maintaining in the incubator at 37° C., 5% CO₂. The stable hACE2 or hDPP4-expressing HeLa cell line was generated using an ACE2 lentivirus protocol previously described (7). Briefly, the pBOB-hACE2 or hDPP4 plasmid and lentiviral packaging plasmids (pMDL, pREV, and pVSV-G (Addgene #12251, #12253, #8454)) were co-transfected into HeLa cells using Lipofectamine 2000 reagent (ThermoFisher Scientific cat. #11668019).

Expression and purification of HCoV S-proteins: To express the soluble human coronavirus (HCoV) S ectodomain proteins with His-tag or with both His- and Avi-tag at the C-terminus, 350 g plasmids in 15 ml Opti-MEM™ (Thermo Fisher Scientific cat. #31985070) were filtered and mixed with 1.8 ml 40K PEI (1 mg/ml) in 15 ml Opti-MEM™ then incubated at room temperature for 30 min and transferred into 1 L FreeStyle293-F cells at the density of 1 million cells/ml. Four days after transfection, the cell cultures were centrifuged at 2500×g for 15 min and filtered through 0.22 m membrane. The His-tagged proteins were purified with the HisPur Ni-NTA Resin (Thermo Fisher Scientific cat. #88221). After washing by wash buffer (25 mM Imidazole, pH 7.4) for at least 3 bed volumes, the protein was eluted with 25 ml elution buffer (250 mM Imidazole, pH 7.4) at slow gravity speed (˜4 sec/drop), then was buffer exchanged into PBS and concentrated using 100K Amicon tubes (Millipore cat. #UFC910024). After being further purified by size-exclusion chromatography by Superdex 200 Increase 10/300 GL column (GE Healthcare cat. #GE28-9909-44), the protein was pooled and concentrated again for further use.

Flow cytometry B cell profiling and monoclonal antibody isolation: Flow cytometry of PBMC samples from infected-vaccinated human donors were conducted following methods described previously (7). 10 ml RPMI1640 medium (Thermo Fisher Scientific, cat. #11875085) with 50% FBS was pre-warmed to 37° C. and used to thaw the frozen PBMC samples, followed by centrifugation at 400×g for 5 min. After discarding supernatant, the cells were resuspended in a 5 ml FACS buffer (PBS, 2% FBS, 2 mM EDTA). Fluorescently labeled antibodies specific for cell surface markers were prepared as 1:100 dilution as a master mix in FACS buffer, to stain the PBMC samples for CD3 (APC-Cy7, BD Pharmingen cat. #557757), CD4 (APC-Cy7, Biolegend cat. #317418), CD8 (APC-Cy7, BD Pharmingen cat. #557760), CD14 (APC-H7, BD Pharmingen cat. #561384), CD19 (PerCP-Cy5.5, Fisher Scientific cat. #NC9963455), CD20 (PerCP-Cy5.5, Biolegend, cat. #302326), IgG (BV605, BD Pharmingen cat. #563246) and IgM (PE, Biolegend, cat. #314508). Meanwhile, SARS-CoV-2 S protein with Avi-tag was conjugated to streptavidin-BV421 (BD Pharmingen cat. #563259) and streptavidin-AF488 (Invitrogen cat. #S11223), respectively, and the MERS-CoV S protein with Avi-tag was conjugated to streptavidin-AF647 (Invitrogen cat. #S21374). After incubating the cells with Ab mixture for cell surface markers for 15 min in dark, S protein-probes were added to the samples and incubated on ice in the dark for 30 min. FVS510 Live/Dead stain (Thermo Fisher Scientific cat. #L34966) in FACS buffer (1:300) was then added to the samples and incubated on ice in the dark for 15 min. After washing with FACS buffer, the stained cells were resuspended in 500 μl of FACS buffer per 10-20 million cells, filtered through the 70-μm mesh cap into FACS tubes (Fisher Scientific cat. #08-771-23) and sorted for S protein-specific memory B cells using BD FACSMelody sorter. In brief, after gating of lymphocytes (SSC-A vs. FSC-A) and singlets (SSC-W vs SSC-H and FSC-H vs. FSC-W), live cells were identified by the negative FVS510 live/dead staining phenotype. The CD3⁻CD4⁻CD8⁻CD14⁻CD19⁺CD20⁺ cells were gated as B cells. By selecting the IgG⁺IgM⁻ population, the cells were sequentially gated for SARS-CoV-2-S-BV421⁺ SARS-CoV-2-S-AF488⁺MERS-CoV-S-AF647⁺ reactivity. Triple positive memory B cells was sorted as single cells into 96-well plates on a cooling platform. Superscript IV Reverse Transcriptase (Invitrogen cat. #18090010), 10 mM dNTPs (Invitrogen cat. #18427088), random hexamers (Gene Link cat. #26-4000-03), Ig gene-specific primers, 0.1 M DTT, RNAseOUT (Invitrogen cat. #10777019), and 10% Igepal (Sigma-Aldrich cat. #18896) were used in the reverse transcription PCR reaction to generate cDNA from the sorted cells right after sorting. Hot Start DNA Polymerases (QIAGEN cat. #203643) and specific primer sets described previously (78, 79) were used to perform two rounds of nested PCR reactions to amplify IgG heavy and light chain variable regions using cDNAs as template. After being purified with SPRI beads according to manufacturer's instructions (Beckman Coulter cat. #B23318), PCR products were constructed into expression vectors encoding human IgG1 or Ig kappa/lambda constant domains, respectively, by Gibson assembly (New England Biolabs cat. #E2621L), then transformed into competent E. coli cells. Single colonies were picked for sequencing and analysis on IMGT V-Quest online tool and downstream plasmid production.

Expression and purification of monoclonal antibodies: Plasmids of the paired heavy and light chains generated after sorting were co-transfected into Expi293F cells to produce monoclonal antibodies. Briefly, 12 g heavy chain plasmid and 12 g of light chain plasmid were added into 3 ml of Opti-MEM™ (Thermo Fisher Scientific cat. #31985070), after inverting, 24 μl of FectoPRO (Polyplus cat. ##116-001) reagent was added into the mixture and inverted. Incubation at room temperature for 10 min was done before adding the mixture into 30 ml of Expi293F cells at 2.8 million cells/ml and incubating in the shaker. 24 hours after transfection, 300 μl of 300 mM sodium valproic acid solution and 275 μl of 45% Glucose solution was used to feed each cell culture. Four days post transfection, supernatants of cell cultures were collected by centrifugation at 2500×g for 15 min and filtering through 0.22 m membrane. Protein A Sepharose (GE Healthcare cat. #45002982) and Protein G Sepharose (GE Healthcare cat. #45000118) were mixed at 1:1 ratio before adding into the supernatant and rotating overnight at 4° C. The solution was then loaded into Econo-Pac columns (BioRad cat. #7321010), washed with 1 column volume of PBS, and antibodies were eluted with 10 ml of 0.2 M citric acid (pH 2.67). The elution was collected into a tube containing 1 ml of 2 M Tris Base solution. 30K Amicon centrifugal filters (Millipore cat. #UFC903024) were used for buffer exchange into PBS and further concentrating into smaller volumes.

ELISA using peptides or recombinant proteins: N-terminal biotinylated peptides corresponding to stem helix of SARS-CoV-1/2, MERS-CoV, HCoV-HKU1, HCoV-OC43, HCoV-229E and HCoV-NL63 were synthesized at A&A Labs (Synthetic Biomolecules) (44). For peptide ELISA, streptavidin (Jackson Immuno Research Labs cat. #016-000-084) was coated at 2 μg/ml in PBS onto 96-well half-area high binding plates (Corning, 3690) overnight at 4° C. For recombinant protein ELISA, mouse anti-His antibody (Invitrogen cat. #MA1-21315-1MG) was used at the same concentration to coat the plates. After washing by 0.05% PBST 3 times, 3% BSA was used to block the plates for 2 h at 37° C. Then 1 g/ml of N-terminal biotinylated peptide or 2 g/ml of His-tagged recombinant spike proteins were applied to plates and incubated for 1 h at RT. After washing by 0.05% PBST 3 times, serially diluted serum samples or antibodies were added into plates and incubated for 1 h at RT. After another washing, alkaline phosphatase-conjugated goat anti-human IgG Fc secondary antibody (Jackson ImmunoResearch cat. #109-055-008) was added in 1:1000 dilution and incubated for 1 h at RT. After the final wash, phosphatase substrate (Sigma-Aldrich cat. #50942-200TAB) dissolved in staining buffer was added into each well. Absorption was measured at 405 nm. Fifty percent maximal response concentrations (EC50) were calculated using the Asymmetrical dose-response model of Richard's version in GraphPad Prism 7 (GraphPad Software). To identify critical residues for antibody binding, single alanine mutations were introduced onto the 25-mer stem helix peptide that comprises the linear epitope. These peptides were synthesized at A&A Labs (Synthetic Biomolecules). ELISA as described above was used to test antibody reactivity against peptides with single alanine substitutions.

Pseudovirus production: HIV-based lentivirus backbone plasmid pCMV-dR8.2 dvpr (Addgene #8455), pBOB-Luciferase (Addgene #170674) were co-transfected into HEK293T cells along with full-length or variously truncated SARS-CoV1, WIV1, SHC014, Pang17, SARS-COV2, SARS-CoV-2 variants of concern [(B.1.1.7(alpha), B.1.351 (beta), P.1 (gamma), B.1.617.2 (delta) and B.1.1.529 (Omicron)] and MERS-CoV spike using Lipofectamine 2000 (ThermoFisher Scientific cat. #11668019) to produce single-round infection-competent pseudoviruses (80). The medium was changed 12-16 hours post transfection. Pseudovirus-containing supernatants were collected 48 hours post transfection and the viral titers were measured by luciferase activity in relative light units (RLU) (Bright-Glo Luciferase Assay System, Promega cat. #E2620). The supernatants were aliquoted and stored at −80° C. until further use.

Neutralization assay: Pseudotyped viral neutralization assay was performed as previously reported (7). In brief, neutralization assays were performed by adding 25 μl of pseudovirus into 25 μl serial dilutions of purified antibodies or plasma from human donors, the mixture was then dispensed into a 96-well plate incubated for one hour at 37° C., then 10,000 HeLa-hACE2 or hDPP4 cells/well (in 50 μl of media containing 20 g/ml Dextran) were directly added to the mixture. After incubation at 37° C. for 42-48 h, luciferase activity was measured. Neutralizing activity was measured by reduction in luciferase activity compared to the virus controls. Fifty percent maximal inhibitory concentrations (IC₅₀), the concentrations required to inhibit infection by 50% compared to the controls, were calculated using the dose-response-inhibition model with 5-parameter Hill slope equation in GraphPad Prism 7 (GraphPad Software).

Neutralization Assay of Replication Competent CoVs: Vero E6 cells (ATCC-C1008) were seeded at 2×10⁴ cells/well in a black-well, black-wall, tissue culture treated, 96-well plate (Corning Cat. #3916) 24 h before the assay. Abs were diluted in MEM supplemented with 5% FBS and 1% Pen/Strep media to obtain an 8-point, 3-fold dilution curve with starting concentration at 20 μg/ml. Eight hundred Pfu of SARS2-nLuc and MERS-nLuc replication competent viruses were mixed with Abs at a 1:1 ratio and incubated at 37° C. for 1 h. One-hundred microliters of virus and Ab mix was added to each well and incubated at 37° C.+5% CO₂ for 20 to 22 h. Luciferase activities were measured by the Nano-Glo Luciferase Assay System (Promega Cat. #N1130) following the manufacturer's protocol using a GloMax luminometer (Promega). Percent inhibition and IC₅₀ were calculated as pseudovirus neutralization assay described above. All experiments were performed as duplicate and independent repeated for three times. All the live virus experiments were performed under biosafety level 3 (BSL-3) conditions at negative pressure, by operators in Tyvek suits wearing personal powered-air purifying respirators.

HEp2 epithelial cell polyreactive assay: According to manufacturer's instructions, HEp2 slides (Hemagen cat. #902360) were used to determine the reactivity of monoclonal antibodies to human epithelial type 2 (HEp2) by indirect immunofluorescence. Briefly, monoclonal antibody was diluted into 50 g/ml by PBS and then added onto immobilized HEp2 slides and incubated for 30 min at RT. After washing by PBS for 3 times, one drop of FITC-conjugated goat anti-human IgG was added onto each well and incubated in the dark for 30 min at RT. After washing, the coverslip was added to HEp2 slide with glycerol and the images were photographed on a Nikon fluorescence microscope for FITC detection.

Polyspecificity reagent (PSR) ELISA: Solubilized CHO cell membrane protein (SMP), human insulin (Sigma-Aldrich cat. #I2643), single strand DNA (Sigma-Aldrich cat. #D8899) were coated onto 96-well half-area high-binding plates (Corning cat. #3690) at 5 μg /ml in PBS overnight at 4° C. After washing with PBST, plates were blocked with 3% BSA for 2 h at 37° C. Antibody samples were diluted at 50 g/ml in 1% BSA with 5-fold serial dilution and then added in plates to incubate for 1 h at room temperature (7). The assay was performed as described in section “ELISA using peptides or recombinant proteins”.

CELISA binding: Flow cytometry-based Cell-ELISA (CELISA) binding of mAbs with HCoV spikes was performed as described previously (43, 81). A total of 4×10⁶ HEK293T cells were seeded into 10 cm round cell culture dishes and incubated at 37° C. After 24 h, HEK293T cells were transfected with plasmids encoding full-length HCoV spikes and were incubated for 36-48 h at 37° C. The cells were harvested and distributed into 96-well round-bottom tissue culture plates for individual staining reactions. For each staining reaction, cells were washed three times with 200 μl FACS buffer (1×PBS, 2% FBS, 1 mM EDTA). The cells were stained for 1 h on ice in 50 μl staining buffer with 10 g/ml of primary antibody. After washing three times with 200 μl FACS buffer, the cells were stained with 50 μl/well of 1:200 diluted R-phycoerythrin (PE)-conjugated mouse anti-human IgG Fc antibody (SouthernBiotech cat. #9040-09) and 1:1000 dilution of Zombie-NIR viability dye (BioLegend cat. #423105) on ice in dark for 45 min. Following three washes with FACS buffer, the cells were resuspended and analyzed by flow cytometry (BD Lyrics cytometer), and the binding data were generated by calculating the Mean Fluorescence Intensity using FlowJo 10 software. Mock-transfected 293T cells were used as a negative control.

BioLayer Interferometry binding (BLI): Octet K2 system (ForteBio) was used to determine the monoclonal antibody binding with S-proteins or selected peptides. IgG was first captured for 60 s by anti-human IgG Fc capture (AHC) biosensors (ForteBio cat. #18-5063), then baseline was provided in Octet buffer (PBS with 0.1% Tween) for another 60 s. After that, the sensors were transferred into wells containing diluted HCoV S-proteins for 120 s for association, and into Octet buffer for disassociation for 240 s. Selected peptides that were N-terminal biotinylated were diluted in Octet buffer and first captured for 60 s by the hydrated streptavidin biosensors (ForteBio cat. #18-5020), then unbound peptides were removed by transferring into Octet buffer for 60 s to provide the baseline. Then the sensors were immersed into monoclonal antibodies in Octet buffer for 120 s for association, followed by transferring into Octet buffer for 240 s for dissociation. The data generated were analyzed using the ForteBio Data Analysis software for correction, and the kinetic curves were fit to 1:1 binding mode. Note that the IgG: spike protomer binding can be a mixed population of 2:1 and 1:1, such that the term ‘apparent affinity’ dissociation constants (K_{D}^{App}) are shown to reflect the binding affinity between IgGs and spike trimers tested.

Antibody immunogenetics analysis: Heavy and light chain sequences of mature antibodies were processed using Diversity Analyzer tool (82). For each CDRH3 translated in the amino acid alphabet, all its k-mers were extracted, where k=2, 3, 4. K-mers appearing in at least 20% of HCDR3 s were reported as motifs. In total 10 motifs were reported for CDRH3 s: AR, ARG, AS, DY, FD, FDY, GS, GV, RG, SS. Each heavy chain sequence was labeled by whether its CDRH3 contains a given motif. The same procedure was applied to CDRL3 s and reported 16 motifs: DS, DSS, FT, GS, PP, QQ, QQY, QY, QYG, SP, SPP, SS, SSP, SSPP, WD, YG. For each CDRH3 motif, the linear regression model was applied to estimate the impact of the motif presence (denoted as “yes” or “no”) and the type of antibody (denoted as “iGL” or “mature”) on the responses of 32 mature antibodies to the stem helix peptides of SARS-CoV-2 and MERS-CoV viruses. The same method was applied to estimate the impact of the presence of LCDR3 motifs. Heavy and light chain sequences of the same antibody were concatenated into a single sequence and collected across all 32 antibodies. The phylogenetic tree derived from the concatenated sequences was constructed using ClusterW2 tool (83) and visualized using the Iroki tool (84).

In vivo virus challenge in mouse model: All mouse experiments were performed at the University of North Carolina, NIH/PHS Animal Welfare Assurance Number: D16-00256 (A3410-01), under approved IACUC protocols. The animal manipulation and virus work was performed in a Class 2A biological safety cabinet in a BSL3 approved facility and workers wore PAPRs, tyvek suites and were double gloved. 12-month-old female Balb/c mice (strain 047) were purchased from Envigo for Sarbecovirus challenge experiments (65, 85). C57B1/6 288/330+/+ mice, which encode two human codons in the mouse dipeptidyl peptidase gene, were used for MERS-CoV mouse adapted challenge experiments (66). Mice were housed in individually ventilated Seal-Safe cages, provided food and water ad libitum and allowed to acclimate at least seven days before experimental use. Twelve hours prior to infection, 300 g antibody was injected into mice intraperitoneally. Immediately prior to infection, mice were anesthetized by injection of ketamine and xylazine intraperitoneally and weighed. Virus (SARS-CoV MA15, SARS-CoV2 MA10 and mouse adapted MERS-CoV-M35c4) was diluted in 50 μl sterile PBS and administered intranasally (65-67, 85). Mice were weighed daily and observed for signs of disease. The mice were euthanized via isoflurane overdose at the designated timepoint, followed by assessment of gross lung pathology and collection of the inferior lobe for virus titration. Respiratory function was measured at day2 post infection via Buxco whole body plethysmography, as previously described (86).

Virus titration: SARS-CoV-2-MA10, SARS-CoV-1-MA15 and MERS-CoV-M35c4 were grown and titered using VeroE6 cells as previously described (87). Briefly, lung tissue was homogenized in 1 ml sterile PBS via Magnalyser (Roche), centrifuged to pellet debris, plated in 10-fold serial dilutions on VeroE6 cells on a 6-well plate and covered with a 1:1 mixture of 1.6% agarose and media. At two (SARS-CoV-1) or three (SARS-CoV-2) days post plating, cells were stained with neutral red and plaques counted.

Statistical Analysis: Statistical analysis was performed using Graph Pad Prism 8, Graph Pad Software, San Diego, California, USA. ID₅₀ or IC₅₀ titers were compared using the non-parametric unpaired Mann-Whitney-U test. The correlation between two groups was determined by Spearman rank test. Groups of data were compared using the Kruskal-Wallis non-parametric test. Dunnett's multiple comparisons test were also performed between experimental groups. Data were considered statistically significant at p<0.05.

### Sequences

The heavy chain (HC) and light chain (LC) variable region sequences of the exemplified S2 stem-helix binding bnAbs are listed below. CDR sequences of these antibodies are shown in Table 1.

```
CC99.103:
HC
  (SEQ ID NO: 1)
QVQLVQSGAEMKKPGASVKISCKASGYTFTSDYMHWVRQAPGQGLEWMGIVNPS
GSGTRYAQKFQGRVTMTRDTPTKTFYIELTRLKSDDTAVYYCASGILTGLFDYWGQ
GTLVTVSS
LC
  (SEQ ID NO: 41)
DVVMTQSPGTLSLSAGERATLSCRASQTMTKNYVAWYQQKPGQAPRLLIYGASTR
ATGIPDRFSGSGSGTDFTLTISRLAPEDFAVYYCLQYGSSPPIFTFGPGTKVEIK
CC68.109:
HC
  (SEQ ID NO: 2)
QVQLVQSGAEVKKPGASVKVSCQASGNTFTNYYVHWVRQAPGQGLEWMGIISPSG
DGTRYAQKFQGRVTMTRDTSTTTVYMELSSLRSEDTAVYYCARGSNWGPWDYWG
QGTLVTVSS
LC
  (SEQ ID NO: 42)
EIVLTQSPGTLSLSPGERATLSCRASQSVRRNYLAWYQQKPGQAPRLLIFGASSRAT
GIPDRFSGSGSGTDFTLTISRLEPEDFAVYYCQQYDSSPPIFTFGPGTKVDIK
CC9.107:
HC
  (SEQ ID NO: 3)
QVQLVESGGGLVRPGRSLRLSCTGSGFNFGDYAMSWFRQVPGTGLEWVGFIRNKA
YGGTPEHAASLRGRFTISRDDSRDIAYLQMNSLTREDTGVYFCATTHAGVSWGQGT
LVTVSS
LC
  (SEQ ID NO: 43)
EIVLTQSPDSLAVSLGETASINCKSSLTVLNPSNNKFHLSWYQQKPGQPPKLLIYWAS
TRESGVPDRFSGSGSGTDFTLTITSLQAEDVAVYYCQQYYSLPITFGQGTRLEIK
CC9.111:
HC
  (SEQ ID NO: 4)
QVQLVQSGAEVKQPGASVKISCKSSEYAFTTYYVHWVRQAPGQGPEWMGLINPSG
GGTSYEQKFRGRVTMTRDTSTGTVYMDLTSLRSEDTAVYYCASGFRGPLFDYWGQ
GTLVTVSS
LC
  (SEQ ID NO: 44)
DVVMTQSPGTLSLSPGERATLSCRASQSVTSNYLAWYQQKPGQAPRLLIYGASTRA
TGIPDRFTGSGSGTDFTLTIRRLEPEDFAVYYCQQYSSSPPRLTFGPGTKVEIK
CC9.113:
HC
  (SEQ ID NO: 5)
QVQLVQSETEVKKPGASVKVSCKASGNTFTSYYFHWVRQAPGQGLEWMGIINPSG
DGTSYAPKFQGRITMTRDTSTNTVYMELSSLRSEDTAVYYCASGFRGPLFDYWGQG
TLVTISS
LC
  (SEQ ID NO: 45)
DVVMTQSPGTLSLSPGERATLSCRASQSVRKNYLAWFQQKPGQAPRLLIYGASSRA
TGIPDRFSGSGSGTDFTLIISRLEPEDFAVYYCQQYDSSPPRLTFGPGTK VEIK
CC9.114:
HC
  (SEQ ID NO: 6)
QVQLVESGGGLVKPGRSLRLSCTGSGFNFGDYAMSWFRQVPGTGLQWVGFIRNKA
YGGTPEHAASLRGRFTISRDDSRDIAYLQMNSLTSEDTGIYFCATTHAGVSWGQGTL
VTVSS
LC
  (SEQ ID NO: 46)
EIVLTQSPDSLAVSLGETASINCKSSLTVLNPSNNKFHLSWYQQKPGQPPKLLIYWAS
TRESGVPDRFSGSGSGTDFTLTITSLQAEDVAVYYCQQYYSLPITFGQGTRLEIK
CC9.116:
HC
  (SEQ ID NO: 7)
QVQLVQSGAEVKKPGASVKVSCKASGYIFSNYYMHWVRQAPGQGLEWMGLINPT
GVNTSYAQKFQGRLTMTRDTSTSTLSMELSNLTSEDTAMYYCSRGSSPWDWGQGT
LVTISS
LC
  (SEQ ID NO: 47)
QPVLTQEPSLTVSPGETVTLTCASSTGAVTSGFYANWFQQKPGQPPRSLIYSSYNKH
SWTPARFSGSLLGGKAALTLSGVQPEDEAEYYCLLYFGAAQLLFGGGTKLTVL
CC9.124:
HC
  (SEQ ID NO: 8)
QVQLVESGGGLVQPGGSLRLSCAASGFPFSNYWMTWVRQAPGKGLEWVANIKRD
GSEKYYVDSVKGRFTISRDNAKNSLDLQMISLRADDTAVYYCATWFGIHWGKGTL
VTVSS
LC
  (SEQ ID NO: 48)
DIVMTQSPDSLAVSLGERATINCKSSQSLLYSSSNKNYLAWFQQKPGQPPKLLIYYA
STRESGVPDRFSGSGSGTDFTLTISSLQAEDVAVYYCQQHYSPPHTFGPGTKVEIK
CC9.130:
HC
  (SEQ ID NO: 9)
EVQLLESGGGMVQPGGSLRLSCAASGFTFSNYAMIWVRQAPGKGLEWVSVISGGG
GGTYYADSVKGRFTISRDNSKNTVFLQMNSLRAEDSAVYYCAKFLQPQHLVFDYW
GRGTLVTVSS
LC
(SEQ ID NO: 49)
DIVMTQSPGTLSLSPGERATLSCRASQRVSNSYLAWYQHKPGQGPRLLMYGASTRA
TGIPDRFSGSGSGTDFTLTISRLEPEDFAVYYCQQYGRSPFTFGPGTKVEIK
CC9.131:
HC
  (SEQ ID NO: 10)
QVQLEQSGAEVKKPGASVKVSCKASGNDFATYYVHWVRQAPGQGLEWMGIINPSG
DGTSYAQKFQGRVTMTRDTSTNRVFMELRSLRSEDTAVYYCASGFTGPLFDYWGQ
GTLVTISS
LC
  (SEQ ID NO: 50)
DIVMTQSPGTLSLSPGERATLSCRASQRVINRYLAWYQQKPGQAPRLLIYGASSRAT
GIPDRFTGSGSETDFTLTIRRLEPEDFAVYYCQHYASSPPRLTFGPGTKLEIK
CC24.105:
HC
(SEQ ID NO: 11)
EVQLVQSGAEVKKPGASVKVSCKVSGNTFTNYYIHWVRQAPGQGLEWQGTIDPSC
GGTRYAQKLQGRVTMTRDTSTKTVYMYVSTLRSEDTAVYYCAMTLYGVFDYWG
QGTLVTVSS
LC
  (SEQ ID NO: 51)
DVVMTQSPGTLSLSPGESATLSCRASQSVRNKYLAWYQQKPGQAPRLLIYGASTRA
TGIPDRFRGSGSETDFTLTISRLEPDDFAVYYCQQYDSSPPSFTFGPGTKLEIK
CC24.107:
HC
  (SEQ ID NO: 12)
QVQLVQSGAEVKKPGASVKVSCKASGNIFTSYYMHWVRQAPGQGLEWLGIINPSG
DGTSYAQKFQGRVTMTKDTSTNTVYMYVSSLRSEDTAVYYCAMTLYGVFDYWGQ
GTLVTVSS
LC
  (SEQ ID NO: 52)
EIVMTQSPGTLSLSPGESATLSCRASQSVRNNYLAWYQQKPGQAPRLLIYGASTRAT
GIPDKFRGSGSGTDFTLTISRLEPDDFAVYYCQQYDSSPPSFTFGPGTKLEIK
CC25.101:
HC
(SEQ ID NO: 13)
EVQLVESGGGLVHPGGSLRLPCAVSGFTFSNYAMSWVRQAPGKGLQWVSVISGGS
GARYYADSVKGRFTISRDNSKNTLYLQMQSLRAEDTAVYYCAKWTHYGDFGVDL
WGRGTLVTVSS
LC
  (SEQ ID NO: 53)
EIVLTQSPGTLSLSPGERATLSCRASHGVSSSSVAWYQQKPGQAPRLLIYDASSRATG
IPDRISGSGSGTDFTLTISRLEAEDFAVYYCQQYGSPPYTFGQGTKLEIK
CC25.103:
HC
  (SEQ ID NO: 14)
QVQLVQSGAEVKKPGASVKVSCKASGYTFTSYYMHWVRQAPGQGLEWMGIITPSG
DNTRYAQKFQGRVTMTRDTSTSTLFMELSSLRSDDTAVYYCASGDSSDSSSYHYW
GQGTLVTVSS
LC
  (SEQ ID NO: 54)
DIVMTQSPSSVSASVGDRVTITCRASQSISRWLAWYQQKPGKAPKLLIYAVSTLQSG
VPSRFSGSRSGTDFTLTISSLQPEDFATYYCQQANSSPPSFTFGPGTKLEIK
CC25.104:
HC
  (SEQ ID NO: 15)
QVQLVQSGAEVKKPGASVKVSCKASGYNFIDHYMQWVRQAPGQGLQWMGIISPSA
GYTAYAQRFQGRVTLTGDTSTSTVYMELRSLRSEDTAIYYCARLRFGVNDHWGQG
TLVTVSS
LC
  (SEQ ID NO: 55)
DVVMTQSPGTLSLSPGERATLSCRASQSVRSNYLAWYQQKPGQAPRLLIYGVSSRA
TGIPDRFSGSGSGTDFTLTISRLEPEDFAVYYCQQYDSSPPIFTFGPGTKLEIK
CC25.105:
HC
  (SEQ ID NO: 16)
QVQLQESGPGLVKPSQTLSLTCTVSGGSISSGGYYWTWIRQHPGKGLEWIGYISYRG
NTFYNPSLKSRAAMSIDTSKNQFSLNLSSVTAADTAVYHCAGTSAGGMGSHAMDV
WGQGTTVTVSS
LC
  (SEQ ID NO: 56)
QSVLTQPPSVSAAPGQKVTISCSGSSSNIGINFVSWYQQLPGTAPKLLIYDNNKRPSGI
PDRFSGSKSGTSATLDITGLQTGDDADYYCGAWDSSLGWVFGGGTKLTVL
CC25.106:
HC
  (SEQ ID NO: 17)
EVQLVQSGAEVKKPGASLKVSCKASGYTFTDYYMHWVRQAPGQGLEWMGIIKPSA
GNTRNAQKFQGRVTMTRDTSTSTVYMELSALRFEDTAVYYCARGGVHGLDYWGQ
GTLVTVSS
LC
  (SEQ ID NO: 57)
QSVLTQPPSVSAPPGQKVTISCSGSSSNIGNNYVSWYQQLPGTAPKLLIHENNQRPSG
IPDRFSGSKSGTSATLGITGLQTGDEADYYCGTWDTNLGAFVFGAATRVTVL
CC25.108:
HC
  (SEQ ID NO: 18)
QVQLVQSGAEVRKPGASVKVSCKASGDTFASNYMLWVRQAPGQGLEWMGIINPSG
DRTSYAQKFQGRVTMTRDTSTSTVYMELRSLRSEDTAVYFCARLRFGVNDYWGQG
TLVTVSS
LC
  (SEQ ID NO: 58)
EIVMTQSPGTLSLSPGDRATLSCRASQSVTSNYLAWYQQKPGQAPRLLIYGASTRAT
GIPDRFSGSGSGTDFTLTISRLEPEDFAVYYCEQYGSSPPIFTFGPGTKVDIK
CC25.112:
HC
  (SEQ ID NO: 19)
QVQLLESGGGLAQPGGSLRLSCAASGFTFSRYAMSWVRQAPGKGLEWVSVTSGGG
GSSYYADSVKGRFTISRDNSKNTLYLQMISLRAEDTAVYYCAKVGTTMVYFDYWG
QGTLVTVSS
LC
  (SEQ ID NO: 59)
EIVLTQSPGTLSLSPGERATLSCRASQSVSSSHLAWYQQKPGQAPRLLIHGASSRATG
IPDRFSGSGSGTDFTLTISRLEPEDFAVYHCQQYGDSPPTFGQGTKVDIK
CC67.105:
HC
(SEQ ID NO: 20)
EVQLLESGGGLVQPGESLRLSCAVSGFTFSSYAMAWVRQAPGKGPEWVSVISGASG
SSYYTDSVKGRFTISRDNSKNTLYLQMNSLRAEDTAVYYCAKVKQVLYFDYWGQG
TLVTVSS
LC
(SEQ ID NO: 60)
EIVLTQSPGTLSLSPGERATLSCRARQSVSSSLAWYQQRPGQAPRLLIYDASTRATGF
PDRFSGSGSGTDFTLTISRLEPEDFAVYYCQQYGSSPPTFGQGTKLEIK
CC67.108:
HC
  (SEQ ID NO: 21)
EVQLVQSGAEVKKPGASVKVSCQASGFTFSNYYMNWVRQAPGRGLEWMGIINPSG
QSTSYPQKFQGRVTMTRDTSTSTVNMELSSLKSEDTAVYYCVRVARGGFDIWGQG
TVVTVSS
LC
(SEQ ID NO: 61)
DVVMTQSPTSLSASVGDRVTITCRASQSLSTYLNWYQQKPGKAPKLLIYEASTLQTG
VPSRFSGSRSGTDFTLTISSLQPEDFATYYCQQSYSSPPMFIFGPGTKLEIK
CC67.130:
HC
(SEQ ID NO: 22)
EVOLVESGGGLVQPGGSLRLSCAVSGLTFSRHAMSWVRQTPGKGLGWVSVISGGG
GGTYYADSVKGRFTISRDNSKNTLYLLMNSLRVEDTAIYYCAKVAGGTVFFDYWG
QGALVTVSS
LC
(SEQ ID NO: 62)
EIVMTQSPGTLSLSPGERATLSCRASQSVNSNYLAWYQQRPGQAPRLLISGASSRAT
DIPDRFSGSGSGTDFALIISRLETEDFAVYYCQQYGGSPPTFGGGTKVDIK
CC68.104:
HC
(SEQ ID NO: 23)
QVQLVQSGAEVKTPGASVKVSCKASGDTFTNYYVHWVRQAPGQGLEWMGIINPSG
YGTTYAQKFQGRVTMTRDTSTGTVYMELSSLKSEDTAVYYCARGSNWGPWDYWG
QGTLVTVSS
LC
(SEQ ID NO: 63)
DIVMTQSPGTLSLSPGERATLSCRASQSVRRNYLAWYQQKPGQAPRLLIFGASSRAT
GIPDRFSGSGSGTDFTLTISRLEPEDFAVYYCQQYDSSPPIFTFGPGTKVEIK
CC9.104:
HC
(SEQ ID NO: 24)
QVQLVQSGAEVKKPGASVKVSCKASGTTFTSDYMHWVRQAPGQGLEWMGIIDPSG
GGTSYARKFQGRVTMTRDTSTSTVYMELGTLRSEDTAVYFCARGSSGWYFWGQGT
LVTVSS
LC
(SEQ ID NO: 64)
EIVMTQSPGTLSLSPGERATLSCRASQNVRRNYLAWYQQKPGQAPRLLIYGASSRAT
GIPDRFSGSGSGTDFTLTISRLEPEDFVVYYCQQYDSSPPIFTFGPGTKVEIK
CC84.102:
HC
  (SEQ ID NO: 25)
QVQLVQSGAEVTKPGASVTVSCKASGYTFTNYYMHWVRQAPGQGLEWMGIIKPSG
GNTIYAQKFQGRVTMTRDTSTSTVYLELSSLRSEDTAVYYCANTMIRGIIITHWGQG
TLVTVSS
LC
  (SEQ ID NO: 65)
QSALTQPPSASGTPGQRVTISCSGSTSNIGRNSVNWYQQLPGTAPKLLMYSNNQRPS
GVPDRFSGSKSGTSASLAISGLQSEDEADYYCAAWDDSLNGSVVFGGGTKLTVL
CC84.115:
HC
(SEQ ID NO: 26)
EVQLVQSGAEVKKPGASVKLSCKASGYTFTTYYMHWVRQAPGQGLEWIGIINPSGA
GTSYAQQFQGRITMTRDTSTSTLYMELSSLRSEDTAVYYCASPPRGSSSALGVWGQ
GTLVTVSS
LC
  (SEQ ID NO: 66)
DIQMTQSPSSVSASVGDRVIITCRASQGISSWLAWYQQKPGKAPKLLIYAASILQSGV
PSRFSGSGSGTDFTLTISSLQPEDFATYYCQQANSFPPIFTFGPGTKLEIK
CC92.133:
HC
  (SEQ ID NO: 27)
QVQLLQSGPEVKRPGASVKVSCKGSADTLSGYYMHWVRQAPGQGLEWMGLITPN
GAGTRYPQNFQGRVTMTRDTSTSAIYVELRSLTFEDTAVYYCARGEDNGSFFLWGR
GTLVTVSS
LC
  (SEQ ID NO: 67)
EIVMTQSPGSLSLSPGESVTLSCRASQSVNSRFFAWYQQKPGQAPRLLMFGPSRRAA
GIPDRFSGSGSGTDFTLTISRLEPEDFAVYYCQQYAASPPMYTFGQGTKVEIK
CC92.147:
HC
  (SEQ ID NO: 28)
QVQLVQSGDEVKRPGASVKVSCKASGSTLSGYYMHWVRQAPGQGLEWVGLINPS
GTGTAYAQKLQGRVTMTRDTSTSTVYMHLSRLRSEDTALYYCARMEDNGSYFLW
GQGTLVTVSS
LC
  (SEQ ID NO: 68)
EIVLTQSPGSLSLSPGESVTLSCRASQSVNSRFFAWYQQKPGQAPRLLMFGPSSRAA
GIPDRFSGSGSGTDFTLTISRLEPEDFAVYYCQQYAASPPMYTFGQGTKLEIK
CC95.102:
HC
  (SEQ ID NO: 29)
QVQLLDSGGTLVQPGGSLRLSCVASGFTFSNYAMSWVRQTPGKGLEWVSVISGRG
GATYYADSVKGRFSISRDNSKNTLFLQMNSLRAEDTAVYYCAKLLMVMVFDHWG
QGTVVTVSS
LC
  (SEQ ID NO: 69)
EIVLTQSPGTLSLSPGERATLSCRASQTISDNSLAWYQQKPGQAPRLLIYGASNRVTG
IPDRFSGFGSGTDFTLTISRLEPEDFAVYYCQQYGSSPPTFGQGTKLEIK
CC95.104:
HC
  (SEQ ID NO: 30)
QVQLHESGPGQVKPSQTLSLTCTVSGASINSGRYYWSWLRQRPGKGLEWIGYIHYT
GSTYYNPSLRSRVVFSIDTSSSQFSLKLTAVTAADTAMYYCASLWRESCTTAGCYPK
DTSLYYFDYWGQGILVTVSS
LC
  (SEQ ID NO: 70)
EIVMTQSPPTLSLSPGERATLSCRASQSVSTNLAWYQQKPGQAPRLLIYGASTRATGI
PARFSGGGSGTDFTLTISSLQSEDFAVYYCGAVTFGQGTKLEIK
CC95.108:
HC
  (SEQ ID NO: 31)
EVQLVQSGTEVRQPGASVRVSCKASGYTFTDSYIHWVRQAPGQGLEWMGIIKPSGG
NTRYAQRFQGRVTMTRDTSTSTVYMELSSLRSEDTAVYYCARDSRGPGIFWGQGTL
VTVSS
LC
  (SEQ ID NO: 71)
QSVLTQPPSVSAAPERKVTISCSGSSSNIGTNFVSWYQQLPGTAPKLLIYENNKRPSGI
PDRFSGSKSGTSATLGITGLQTGDEADYYCGAWDSTPGTWVFGGGTRLTVL
CC95.109:
HC
  (SEQ ID NO: 32)
EVQLVQSGAEVKKPGASVKISCKASGYTFSSSYMYWVRQAPGQGLEWMGIIKPSGG
NTRYAQKFQGRVTMTWDTSTSTVYMELSSLTSDDTAVYYCAGDLRGVGGSWGQG
TLVTVSS
LC
  (SEQ ID NO: 72)
QSVLTQPPSVSAAPGQRVTISCSGSSSNIGNNFVSWYQQFPGTAPKLLIYENNKRPSQ
IPDRFSGSKSGTSATLGITGLQTGDEADYYCGTWDTSVGAWVFGGGTKLTVL
CC95.110:
HC
  (SEQ ID NO: 33)
EVQLVQSGAEVKKPGASVKVSCEASGYTLTDYYLHWVRQAPGQGLEWMGIIKPSG
GNTIYAQTLRGRVTMTRDTSASKVYMELRSLRHDDTAVYFCARGGRHAHDIWGQG
TMVTVSS
LC
  (SEQ ID NO: 73)
QSVLTQPPSASAAPGQRVAISCSGSSSNIGSNFVSWYQHLPGTAPKLLISENDQRPSG
VPDRFSGSRSGTSATLGIAGLQAGDEADYYCGTWDTSLGEWVFGGGTKLTVL
CC95.116:
HC
  (SEQ ID NO: 34)
EVOLVESGGGLVKPGGSLRLSCAASGLPFSGAWMTWIRQAPGKAPEWVGRIKSKSD
GGTIDYAAPVKGRFTISRDDSRNTVFLHMDSLKVEDTAVYYCNWNLDYWGQGTLV
TVSS
LC
  (SEQ ID NO: 74)
DIVMTQSPDSLAVSLGERATINCKSSQSILHSSNKKNYLAWYQQKPGQPPKLIIYWA
STRESGVPDRFSGSGSGTDFTLTINSLQAEDVAVYYCHQYYSSRTFGQGTKLEIK
CC95.118:
HC
  (SEQ ID NO: 35)
QVQLVQSGAEVKKPGASVKVSCKASGFTFSDYYMNWVRQAPGQGPEWMGIINPSG
TGTVYAQKFQGRVTMTRDTSTSTVYMELSSLTSEDTAVYYCARVVRGASSLWGQG
TLVTVSS
LC
  (SEQ ID NO: 75)
DIQMTQSPSSVSASVGDRVIITCRASQVISNWLAWYQQKPGKAPNLLIYAVSNLQSG
VPSRFSGSGSGTNFTLTISSLQPEDFATYFCQQASGFPPLFTFGPGTKVDIK
CC95.121:
HC
(SEQ ID NO: 36)
QVQLQESGPGLVKPSQTLSLTCTVSGSGGSISSGGYYWNWIRQHPGKGLEWIGYIHY
SGSTYYHPSLKSRITISVDTSKNQFSLKLSSVTAADTAVYYCARGFRENYDNSGYSS
YYFDYWGQGTLVTVSS
LC
  (SEQ ID NO: 76)
DIQMTQSPSFLSASVGDRVTITCRASQGISSYLAWYQQKPGKTPKLLIYAASTLQSGV
PSRFSGSGSGTEFTLTISSLQPEDFATYFCQQLVAFGQGTKLEIK
CC95.122:
HC
  (SEQ ID NO: 37)
EVQLVQSGAEVRKPGASVKLSCKTSGYTFTNFYIHWVRQAPGQGLEWMGIISTSAG
STRYTQKFQGRVTMTRDTSTSTVYMELSSLRYDDTALYFCARDLYGSRNFHYWGQ
GTLVTVSS
LC
  (SEQ ID NO: 77)
QSVLTQPPSVSAAPGQKVTISCSGSSSNIGENYVSWFLQLPGTAPKVVIYENNNRPSE
IPDRFSGSKSGTSATLGITGLQPGDEADYYCGSWDSSLSVWVFGGGTKLTVL
CC9.106:
HC
  (SEQ ID NO: 38)
QVQLVQSGAEVKKPGASVKVSCKASGTTFTSDYMHWVRQAPGQGLEWMGIIDPSG
GGTSYAQKFQGRVTMTRDTSTSTVYLELSTLRSEDTAVYFCARGSSGWYFWGQGT
LVSVSS
LC
  (SEQ ID NO: 78)
DVVMTQSPGTLSLSPGERATLSCRASQSVRRNYLAWYQQKPGQAPRLLIYGASSRA
TGIPDRFSGSGSGTDFTLTISRLEPEDFAVYYCQQYDSSPPIFTFGPGTKVEIK
CC99.104:
HC
(SEQ ID NO: 39)
EVQLVQSGAEMKKPGASVKISCKASGYTFTSDYMHWVRQAPGQGLEWMGIINPSG
SGTRYAQKFQGRVTMTRDTPTTTFYMELTRLRSEDTAVYYCASGILTGLFDYWGQ
GTLVTVSS
LC
(SEQ ID NO: 79)
DVVMTQSPGTLSLSAGERATLSCRASQSITNNYLAWFQQKPGQAPRLLIYGASRRAT
GIPDRFSGSGSGTDFTLTISRLAPEDFAVYYCLQYGSSPPIFTFGPGTKVEIK
CC99.105:
HC
(SEQ ID NO: 40)
EVQLVQSGAEMKKPGASVKISCKASEYTFTNDYMHWVRQAPGQGLEWMGIINPSG
SGTRYAQKFQGRVTMTRDTPTNTFYIELTRLRSEDTAVYYCASGILTGLFDYWGQG
TLVTVSS
LC
  (SEQ ID NO: 80)
DIVMTQSPGTLSLSPGERATLSCRASQTITNNYLAWYQQKPGQAPRLLIYGASRRAT
GIPDRFSGSGSGTDFTLTISRLAPEDFAVYYCLQYGSSPPIFTFGPGTKVEIK
```

TABLE 1
CDR sequences of 40 S2 binding bnAbs

| | HCDR1 | HCDR2 | HCDR3 | | LCDR2 | LCDR3 |
|---|---|---|---|---|---|---|
| | (SEQ | (SEQ ID | (SEQ ID | LCDR1 | (SEQ | (SEQ |
| Antibody | ID NO:) | NO:) | NO:) | (SEQ ID NO:) | ID NO:) | ID NO:) |
| CC99.103 | GYTFTSDY | VNPSGSGT | ASGILTGL | QTMTKNY (84) | GAS | LQYGSSPPIF |
| | (81) | (82) | FDY (83) | | | T (85) |
| CC68.109 | GNTFTNYY | ISPSGDGT | ARGSNWG | QSVRRNY (89) | GAS | QQYDSSPPIF |
| | (86) | (87) | PWDY (88) | | | T (90) |
| CC9.107 | GFNFGDYA | IRNKAYGG | ATTHAGV | LTVLNPSNNKFH | WAS | QQYYSLPIT |
| | (91) | TP (92) | S (93) | (94) | | (95) |
| CC9.111 | EYAFTTYY | INPSGGGT | ASGFRGPL | QSVTSNY (99) | GAS | QQYSSSPPR |
| | (96) | (97) | FDY (98) | | | LT (100) |
| CC9.113 | GNTFTSYY | INPSGDGT | ASGFRGPL | QSVRKNY (104) | GAS | QQYDSSPPR |
| | (101) | (102) | FDY (103) | | | LT (105) |
| CC9.114 | GFNFGDYA | IRNKAYGG | ATTHAGV | LTVLNPSNNKFH | WAS | QQYYSLPIT |
| | (106) | TP (107) | S (108) | (109) | | (110) |
| CC9.116 | GYIFSNYY | INPTGVNT | SRGSSPW | TGAVTSGFY (114) | SSY | LLYFGAAQL |
| | (111) | (112) | D (113) | | | L (115) |
| CC9.124 | GFPFSNYW | IKRDGSEK | ATWFGIH | QSLLYSSSNKNY | YAS | QQHYSPPHT |
| | (116) | (117) | (118) | (119) | | (120) |
| CC9.130 | GFTFSNYA | ISGGGGGT | AKFLQPQ | QRVSNSY (124) | GAS | QQYGRSPFT |
| | (121) | (122) | HLVFDY | | | (125) |
| | | | (123) | | | |
| CC9.131 | GNDFATYY | INPSGDGT | ASGFTGPL | QRVINRY (129) | GAS | QHYASSPPR |
| | (126) | (127) | FDY (128) | | | LT (130) |
| CC24.105 | GNTFTNYY | IDPSCGGT | AMTLYGV | QSVRNKY (134) | GAS | QQYDSSPPS |
| | (131) | (132) | FDY (133) | | | FT (135) |
| CC24.107 | GNIFTSYY | INPSGDGT | AMTLYGV | QSVRNNY (139) | GAS | QQYDSSPPS |
| | (136) | (137) | FDY (138) | | | FT (140) |
| CC25.101 | GFTFSNYA | ISGGSGAR | AKWTHYG | HGVSSSS (144) | DAS | QQYGSPPYT |
| | (141) | (142) | DFGVDL | | | (145) |
| | | | (143) | | | |
| CC25.103 | GYTFTSYY | ITPSGDNT | ASGDSSDS | QSISRW (149) | AVS | QQANSSPPS |
| | (146) | (147) | SSYHY | | | FT (150) |
| | | | (148) | | | |
| CC25.104 | GYNFIDHY | ISPSAGYT | ARLRFGV | QSVRSNY (154) | GVS | QQYDSSPPIF |
| | (151) | (152) | NDH (153) | | | T (155) |
| CC25.105 | GGSISSGGY | ISYRGNT | AGTSAGG | SSNIGINF (159) | DNN | GAWDSSLG |
| | Y (156) | (157) | MGSHAM | | | WV (160) |
| | | | DV (158) | | | |
| CC25.106 | GYTFTDYY | IKPSAGNT | ARGGVHG | SSNIGNNY (164) | ENN | GTWDTNLG |
| | (161) | (162) | LDY (163) | | | AFV (165) |
| CC25.108 | GDTFASNY | INPSGDRT | ARLRFGV | QSVTSNY (169) | GAS | EQYGSSPPIF |
| | (166) | (167) | NDY (168) | | | T (170) |
| CC25.112 | GFTFSRYA | TSGGGGSS | AKVGTTM | QSVSSSH (174) | GAS | QQYGDSPPT |
| | (171) | (172) | VYFDY | | | (175) |
| | | | (173) | | | |
| CC67.105 | GFTFSSYA | ISGASGSS | AKVKQVL | QSVSSS (179) | DAS | QQYGSSPPT |
| | (176) | (177) | YFDY (178) | | | (180) |
| CC67.108 | GFTFSNYY | INPSGQST | VRVARGG | QSLSTY (184) | EAS | QQSYSSPPM |
| | (181) | (182) | FDI (183) | | | FI (185) |
| CC67.130 | GLTFSRHA | ISGGGGGT | AKVAGGT | QSVNSNY (189) | GAS | QQYGGSPPT |
| | (186) | (187) | VFFDY | | | (190) |
| | | | (188) | | | |
| CC68.104 | GDTFTNYY | INPSGYGT | ARGSNWG | QSVRRNY (194) | GAS | QQYDSSPPIF |
| | (191) | (192) | PWDY | | | T (195) |
| | | | (193) | | | |
| CC9.104 | GTTFTSDY | IDPSGGGT | ARGSSGW | QNVRRNY (199) | GAS | QQYDSSPPIF |
| | (196) | (197) | YF (198) | | | T (200) |
| CC84.102 | GYTFTNYY | IKPSGGNT | ANTMIRGI | TSNIGRNS (204) | SNN | AAWDDSLN |
| | (201) | (202) | IITH (203) | | | GSVV (205) |
| CC84.115 | GYTFTTYY | INPSGAGT | ASPPRGSS | QGISSW (209) | AAS | QQANSFPPIF |
| | (206) | (207) | SALGV | | | T (210) |
| | | | (208) | | | |
| CC92.133 | ADTLSGYY | ITPNGAGT | ARGEDNG | QSVNSRF (214) | GPS | QQYAASPP |
| | (211) | (212) | SFFL (213) | | | MYT (215) |
| CC92.147 | GSTLSGYY | INPSGTGT | ARMEDNG | QSVNSRF (219) | GPS | QQYAASPP |
| | (216) | (217) | SYFL (218) | | | MYT (220) |
| CC95.102 | GFTFSNYA | ISGRGGAT | AKLLMVM | QTISDNS (224) | GAS | QQYGSSPPT |
| | (221) | (222) | VFDH (223) | | | (225) |
| CC95.104 | GASINSGRY | IHYTGST | ASLWRES | QSVSTN (229) | GAS | GAVT (230) |
| | Y (226) | (227) | CTTAGCY | | | |
| | | | PKDTSLY | | | |
| | | | YFDY (228) | | | |
| CC95.108 | GYTFTDSY | IKPSGGNT | ARDSRGP | SSNIGTNF (234) | ENN | GAWDSTPG |
| | (231) | (232) | GIF (233) | | | TWV (235) |
| CC95.109 | GYTFSSSY | IKPSGGNT | AGDLRGV | SSNIGNNF (239) | ENN | GTWDTSVG |
| | (236) | (237) | GGS (238) | | | AWV (240) |
| CC95.110 | GYTLTDYY | IKPSGGNT | ARGGRHA | SSNIGSNF (244) | END | GTWDTSLG |
| | (241) | (242) | HDI (243) | | | EWV (245) |
| CC95.116 | GLPFSGAW | IKSKSDGGT | NWNLDY | QSILHSSNKKNY | WAS | HQYYSSRT |
| | (246) | I (247) | (248) | (249) | | (250) |
| CC95.118 | GFTFSDYY | INPSGTGT | ARVVRGA | QVISNW (254) | AVS | QQASGFPPL |
| | (251) | (252) | SSL (253) | | | FT (255) |
| CC95.121 | GGSISSGGY | IHYSGST | ARGFREN | QGISSY (259) | AAS | QQLVA (260) |
| | Y (256) | (257) | YDNSGYS | | | |
| | | | SYYFDY | | | |
| | | | (258) | | | |
| CC95.122 | GYTFTNFY | ISTSAGST | ARDLYGS | SSNIGENY (264) | ENN | GSWDSSLSV |
| | (261) | (262) | RNFHY | | | WV (265) |
| | | | (263) | | | |
| CC9.106 | GTTFTSDY | IDPSGGGT | ARGSSGW | QSVRRNY (269) | GAS | QQYDSSPPIF |
| | (266) | (267) | YF (268) | | | T (270) |
| CC99.104 | GYTFTSDY | INPSGSGT | ASGILTGL | QSITNNY (274) | GAS | LQYGSSPPIF |
| | (271) | (272) | FDY (273) | | | T (275) |
| CC99.105 | EYTFTNDY | INPSGSGT | ASGILTGL | QTITNNY (279) | GAS | LQYGSSPPIF |
| | (276) | (277) | FDY (278) | | | T (280) |

### REFERENCES

1. L. R. Baden et al., Efficacy and Safety of the mRNA-1273 SARS-CoV-2 Vaccine. The New England journal of medicine 384, 403-416 (2020).
2. F. P. Polack et al., Safety and Efficacy of the BNT162b2 mRNA Covid-19 Vaccine. The New England journal of medicine 383, 2603-2615 (2020).
3. Z. Wang et al., mRNA vaccine-elicited antibodies to SARS-CoV-2 and circulating variants. Nature 592, 616-622 (2021).
4. P. B. Gilbert et al., Title: Immune Correlates Analysis of the mRNA-1273 COVID-19 Vaccine Efficacy Trial. Science 375, 43-50 (2021).
5. A. J. Greaney et al., Antibodies elicited by mRNA-1273 vaccination bind more broadly to the receptor binding domain than do those from SARS-CoV-2 infection. Science translational medicine 13, eabi9915 (2021).
6. C. O. Barnes et al., Structures of Human Antibodies Bound to SARS-CoV-2 Spike Reveal Common Epitopes and Recurrent Features of Antibodies. Cell 182, 828-842 e816 (2020).
7. T. F. Rogers et al., Isolation of potent SARS-CoV-2 neutralizing antibodies and protection from disease in a small animal model. Science 369, 956-963 (2020).
8. M. Yuan et al., Structural basis of a shared antibody response to SARS-CoV-2. Science 369, 1119-1123 (2020).
9. D. F. Robbiani et al., Convergent antibody responses to SARS-CoV-2 in convalescent individuals. Nature 584, 437-442 (2020).
10. P. J. M. Brouwer et al., Potent neutralizing antibodies from COVID-19 patients define multiple targets of vulnerability. Science 369, 643-650 (2020).
11. S. J. Zost et al., Potently neutralizing and protective human antibodies against SARS-CoV-2. Nature 584, 443-449 (2020).
12. C. O. Barnes et al., SARS-CoV-2 neutralizing antibody structures inform therapeutic strategies. Nature 588, 682-687 (2020).
13. Z. Wang et al., Naturally enhanced neutralizing breadth against SARS-CoV-2 one year after infection. Nature 595, 426-431 (2021).
14. M. Yuan et al., Structural and functional ramifications of antigenic drift in recent SARS-CoV-2 variants. Science 373, 818-823 (2021).
15. P. Wang et al., Antibody Resistance of SARS-CoV-2 Variants B.1.351 and B.1.1.7. Nature 593, 130-135 (2021).
16. J. R. Mascola, B. S. Graham, A. S. Fauci, SARS-CoV-2 Viral Variants-Tackling a Moving Target. JAMA 325, 1261-1262 (2021).
17. W. T. Harvey et al., SARS-CoV-2 variants, spike mutations and immune escape. Nat Rev Microbiol 19, 409-424 (2021).
18. E. Cameroni et al., Broadly neutralizing antibodies overcome SARS-CoV-2 Omicron antigenic shift. Nature, (2021).
19. L. Liu et al., Striking Antibody Evasion Manifested by the Omicron Variant of SARS-CoV-2. Nature, (2021).
20. S. Cele et al., Omicron extensively but incompletely escapes Pfizer BNT162b2 neutralization. Nature, (2021).
21. D. Planas et al., Considerable escape of SARS-CoV-2 Omicron to antibody neutralization. Nature, (2021).
22. J. M. Carreno et al., Activity of convalescent and vaccine serum against SARS-CoV-2 Omicron. Nature, (2021).
23. W. F. Garcia-Beltran et al., mRNA-based COVID-19 vaccine boosters induce neutralizing immunity against SARS-CoV-2 Omicron variant. Cell, (2022).
24. C. K. Wibmer et al., SARS-CoV-2 501Y.V2 escapes neutralization by South African COVID-19 donor plasma. Nature medicine 27, 622-625 (2021).
25. Y. Cao et al., Omicron escapes the majority of existing SARS-CoV-2 neutralizing antibodies. Nature, (2021).
26. M. Letko, A. Marzi, V. Munster, Functional assessment of cell entry and receptor usage for SARS-CoV-2 and other lineage B betacoronaviruses. Nat Microbiol 5, 562-569 (2020).
27. V. D. Menachery et al., SARS-like WIV1-CoV poised for human emergence. Proceedings of the National Academy of Sciences of the United States of America 113, 3048-3053 (2016).
28. A. M. Zaki, S. van Boheemen, T. M. Bestebroer, A. D. Osterhaus, R. A. Fouchier, Isolation of a novel coronavirus from a man with pneumonia in Saudi Arabia. The New England journal of medicine 367, 1814-1820 (2012).
29. D. R. Burton, E. J. Topol, Variant-proof vaccines—invest now for the next pandemic. Nature 590, 386-388 (2021).
30. W. C. Koff, S. F. Berkley, A universal coronavirus vaccine. Science 371, 759 (2021).
31. D. M. Morens, J. K. Taubenberger, A. S. Fauci, Universal Coronavirus Vaccines—An Urgent Need. The New England journal of medicine, (2021).
32. T. N. Starr et al., SARS-CoV-2 RBD antibodies that maximize breadth and resistance to escape. Nature 597, 97-102 (2021).
33. M. A. Tortorici et al., Broad sarbecovirus neutralization by a human monoclonal antibody. Nature 597, 103-108 (2021).
34. D. Pinto et al., Cross-neutralization of SARS-CoV-2 by a human monoclonal SARS-CoV antibody. Nature 583, 290-295 (2020).
35. W.-t. He et al., Targeted isolation of panels of diverse human broadly neutralizing antibodies against Betacoronaviruses. bioRxiv, 2021.2009.2008.459480 (2021).
36. D. R. Martinez et al., A broadly cross-reactive antibody neutralizes and protects against sarbecovirus challenge in mice. Science translational medicine, eabj7125 (2021).
37. M. Yuan et al., A highly conserved cryptic epitope in the receptor binding domains of SARS-CoV-2 and SARS-CoV. Science 368, 630-633 (2020).
38. C. A. Jette et al., Broad cross-reactivity across sarbecoviruses exhibited by a subset of COVID-19 donor-derived neutralizing antibodies. bioRxiv, (2021).
39. W.-t. He et al., Broadly neutralizing antibodies to SARS-related viruses can be readily induced in rhesus macaques. bioRxiv, 2021.2007.2005.451222 (2021).
40. D. Li et al., In vitro and in vivo functions of SARS-CoV-2 infection-enhancing and neutralizing antibodies. Cell 184, 4203-4219 e4232 (2021).
41. C. G. Rappazzo et al., Broad and potent activity against Betacoronaviruses by an engineered human monoclonal antibody. Science 371, 823-829 (2021).
42. L. Dai, G. F. Gao, Viral targets for vaccines against COVID-19. Nature reviews. Immunology 21, 73-82 (2020).
43. G. Song et al., Cross-reactive serum and memory B-cell responses to spike protein in SARS-CoV-2 and endemic coronavirus infection. Nature communications 12, 2938 (2021).
44. P. Zhou et al., A protective broadly cross-reactive human antibody defines a conserved site of vulnerability on beta-coronavirus spikes. bioRxiv, (2021).
45. D. Pinto et al., Broad betacoronavirus neutralization by a stem helix-specific human antibody. Science 373, 1109-1116 (2021).
46. C. L. Hsieh et al., Stabilized coronavirus spike stem elicits a broadly protective antibody. Cell reports 37, 109929 (2021).
47. N. K. Hurlburt et al., Structural definition of a pan-sarbecovirus neutralizing epitope on the spike S2 subunit. bioRxiv, 2021.2008.2002.454829 (2021).
48. M. M. Sauer et al., Structural basis for broad coronavirus neutralization. Nature structural & molecular biology 28, 478-486 (2021).
49. W. Li et al., Structural basis and mode of action for two broadly neutralizing antibodies against SARS-CoV-2 emerging variants of concern. Cell reports 38, 110210 (2022).
50. C. Wang et al., A conserved immunogenic and vulnerable site on the coronavirus spike protein delineated by cross-reactive monoclonal antibodies. Nature communications 12, 1715 (2021).
51. M. F. Jennewein et al., Isolation and characterization of cross-neutralizing coronavirus antibodies from COVID-19+ subjects. Cell reports 36, 109353 (2021).
52. R. Andrabi, J. N. Bhiman, D. R. Burton, Strategies for a multi-stage neutralizing antibody-based HIV vaccine. Curr Opin Immunol 53, 143-151 (2018).
53. J. M. Steichen et al., A generalized HIV vaccine design strategy for priming of broadly neutralizing antibody responses. Science 366, (2019).
54. J. Jardine et al., Rational HIV immunogen design to target specific germline B cell receptors. Science 340, 711-716 (2013).
55. E. J. Erbelding et al., A Universal Influenza Vaccine: The Strategic Plan for the National Institute of Allergy and Infectious Diseases. J Infect Dis 218, 347-354 (2018).
56. D. R. Burton, L. M. Walker, Rational Vaccine Design in the Time of COVID-19. Cell host & microbe 27, 695-698 (2020).
57. A. C. Hurt, A. K. Wheatley, Neutralizing Antibody Therapeutics for COVID-19. Viruses 13, 628 (2021).
58. L. A. Jackson et al., An mRNA Vaccine against SARS-CoV-2—Preliminary Report. The New England journal of medicine 383, 1920-1931 (2020).
59. J. Sadoff et al., Safety and Efficacy of Single-Dose Ad26.COV2.S Vaccine against Covid-19. The New England journal of medicine 384, 2187-2201 (2021).
60. C. Soto et al., High frequency of shared clonotypes in human B cell receptor repertoires. Nature 566, 398-402 (2019).
61. B. Briney, A. Inderbitzin, C. Joyce, D. R. Burton, Commonality despite exceptional diversity in the baseline human antibody repertoire. Nature 566, 393-397 (2019).
62. M. Gidoni et al., Mosaic deletion patterns of the human antibody heavy chain gene locus shown by Bayesian haplotyping. Nature communications 10, 628 (2019).
63. R. Andrabi et al., Identification of Common Features in Prototype Broadly Neutralizing Antibodies to HIV Envelope V2 Apex to Facilitate Vaccine Design. Immunity 43, 959-973 (2015).
64. R. Andrabi et al., The Chimpanzee SIV Envelope Trimer: Structure and Deployment as an HIV Vaccine Template. Cell reports 27, 2426-2441 e2426 (2019).
65. S. R. Leist et al., A Mouse-Adapted SARS-CoV-2 Induces Acute Lung Injury and Mortality in Standard Laboratory Mice. Cell 183, 1070-1085 e1012 (2020).
66. A. S. Cockrell et al., A mouse model for MERS coronavirus-induced acute respiratory distress syndrome. Nat Microbiol 2, 16226 (2016).
67. M. G. Douglas, J. F. Kocher, T. Scobey, R. S. Baric, A. S. Cockrell, Adaptive evolution influences the infectious dose of MERS-CoV necessary to achieve severe respiratory disease. Virology 517, 98-107 (2018).
68. M. S. Cohen, Monoclonal Antibodies to Disrupt Progression of Early Covid-19 Infection. The New England journal of medicine 384, 289-291 (2021).
69. W. A. Fischer, 2nd et al., A Phase 2a clinical trial of Molnupiravir in patients with COVID-19 shows accelerated SARS-CoV-2 RNA clearance and elimination of infectious virus. Science translational medicine, eab17430 (2021).
70. A. Schafer et al., Antibody potency, effector function, and combinations in protection and therapy for SARS-CoV-2 infection in vivo. The Journal of experimental medicine 218, (2021).
71. U. Greenbaum et al., High Levels of Common Cold Coronavirus Antibodies in Convalescent Plasma Are Associated With Improved Survival in COVID-19 Patients. Frontiers in immunology 12, 675679 (2021).
72. P. Kaplonek et al., Early cross-coronavirus reactive signatures of humoral immunity against COVID-19. Sci Immunol 6, eabj2901 (2021).
73. B. F. Haynes, J. R. Mascola, The quest for an antibody-based HIV vaccine. Immunological reviews 275, 5-10 (2017).
74. P. D. Kwong, J. R. Mascola, G. J. Nabel, Broadly neutralizing antibodies and the search for an HIV-1 vaccine: the end of the beginning. Nature reviews. Immunology 13, 693-701 (2013).
75. K. O. Saunders et al., Neutralizing antibody vaccine for pandemic and pre-emergent coronaviruses. Nature 594, 553-559 (2021).
76. A. C. Walls et al., Elicitation of Potent Neutralizing Antibody Responses by Designed Protein Nanoparticle Vaccines for SARS-CoV-2. Cell 183, 1367-1382 e1317 (2020).
77. M. G. Joyce et al., A SARS-CoV-2 ferritin nanoparticle vaccine elicits protective immune responses in nonhuman primates. Science translational medicine, eabi5735 (2021).
78. T. Tiller et al., Efficient generation of monoclonal antibodies from single human B cells by single cell RT-PCR and expression vector cloning. Journal of immunological methods 329, 112-124 (2008).
79. N. A. Doria-Rose et al., New Member of the V1V2-Directed CAP256-VRC26 Lineage That Shows Increased Breadth and Exceptional Potency. Journal of virology 90, 76-91 (2016).
80. X. Zhou et al., Diverse immunoglobulin gene usage and convergent epitope targeting in neutralizing antibody responses to SARS-CoV-2. Cell reports 35, 109109 (2021).
81. P. Zhou et al., Broadly resistant HIV-1 against CD4-binding site neutralizing antibodies. PLoS pathogens 15, e1007819 (2019).
82. A. Shlemov et al., Reconstructing Antibody Repertoires from Error-Prone Immunosequencing Reads. Journal of immunology 199, 3369-3380 (2017).
83. M. A. Larkin et al., Clustal W and Clustal X version 2.0. Bioinformatics 23, 2947-2948 (2007).
84. R. M. Moore, A. O. Harrison, S. M. McAllister, S. W. Polson, K. E. Wommack, Iroki: automatic customization and visualization of phylogenetic trees. PeerJ 8, e8584 (2020).
85. M. Frieman et al., Molecular determinants of severe acute respiratory syndrome coronavirus pathogenesis and virulence in young and aged mouse models of human disease. Journal of virology 86, 884-897 (2012).
86. V. D. Menachery, L. E. Gralinski, R. S. Baric, M. T. Ferris, New Metrics for Evaluating Viral Respiratory Pathogenesis. PloS one 10, e0131451 (2015).
87. B. Yount et al., Severe acute respiratory syndrome coronavirus group-specific open reading frames encode nonessential functions for replication in cell cultures and mice. Journal of virology 79, 14909-14922 (2005).
88. E. Q. Wang et al., Assessing the Potential Risk of Cross-Reactivity Between Anti-Bococizumab Antibodies and Other Anti-PCSK9 Monoclonal Antibodies. BioDrugs 33, 571-579 (2019).
89. P. Niu et al., Ultrapotent Human Neutralizing Antibody Repertoires Against Middle East Respiratory Syndrome Coronavirus From a Recovered Patient. J Infect Dis 218, 1249-1260 (2018).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

1. A pharmaceutical composition comprising (a) a therapeutically effective amount of an antibody or antigen-binding fragment thereof that specifically binds to the S2 stem helix of a betacoronavirus, and (b) a second moiety fused to the antibody or antigen-binding fragment; or a pharmaceutically acceptable carrier; wherein the antibody or antigen-binding fragment comprises: (i) heavy chain CDR sequences (HCDR1-3) as set forth respectively in (1) SEQ ID NOs:81-83, (2) SEQ ID NOs:86-88, (3) SEQ ID NOs:91-93, (4) SEQ ID NOs:96-98, (5) SEQ ID NOs:101-103, (6) SEQ ID NOs:106-108, (7) SEQ ID NOs:111-113, (8) SEQ ID NOs:116-118, (9) SEQ ID NOs:121-123, (10) SEQ ID NOs:126-128, (11) SEQ ID NOs:131-133, (12) SEQ ID NOs:136-138, (13) SEQ ID NOs:141-143, (14) SEQ ID NOs:146-148, (15) SEQ ID NOs:151-153, (16) SEQ ID NOs:156-158, (17) SEQ ID NOs:161-163, (18) SEQ ID NOs:166-168, (19) SEQ ID NOs:171-173, (20) SEQ ID NOs:176-178, (21) SEQ ID NOs:181-183, (22) SEQ ID NOs:186-188, (23) SEQ ID NOs:191-193, (24) SEQ ID NOs:196-198, (25) SEQ ID NOs:201-203, (26) SEQ ID NOs:206-208, (27) SEQ ID NOs:211-213, (28) SEQ ID NOs:216-218, (29) SEQ ID NOs:221-223, (30) SEQ ID NOs:226-228, (31) SEQ ID NOs:231-233, (32) SEQ ID NOs:236-238, (33) SEQ ID NOs:241-243, (34) SEQ ID NOs:246-248, (35) SEQ ID NOs:251-253, (36) SEQ ID NOs:256-258, (37) SEQ ID NOs:261-263, (38) SEQ ID NOs:266-268, (39) SEQ ID NOs:271-273, or (40) SEQ ID NOs:276-278; and (ii) light chain CDR sequences (LCDR1-3) as set forth respectively in (1) SEQ ID NO:84, GAS, and SEQ ID NO:85, (2) SEQ ID NO:89, GAS, and SEQ ID NO:90, (3) SEQ ID NO:94, WAS, and SEQ ID NO:95, (4) SEQ ID NO:99, GAS, and SEQ ID NO:100, (5) SEQ ID NO:104, GAS, and SEQ ID NO:105, (6) SEQ ID NO:109, WAS, and SEQ ID NO: 110, (7) SEQ ID NO: 114, SSY, and SEQ ID NO: 115, (8) SEQ ID NO:119, YAS, and SEQ ID NO:120, (9) SEQ ID NO:124, GAS, and SEQ ID NO:125, (10) SEQ ID NO:129, GAS, and SEQ ID NO:130, (11) SEQ ID NO:134, GAS, and SEQ ID NO:135, (12) SEQ ID NO:139, GAS, and SEQ ID NO:140, (13) SEQ ID NO:144, DAS, and SEQ ID NO:145, (14) SEQ ID NO:149, AVS, and SEQ ID NO:150, (15) SEQ ID NO:154, GVS, and SEQ ID NO:155, (16) SEQ ID NO:159, DNN, and SEQ ID NO:160, (17) SEQ ID NO:164, ENN, and SEQ ID NO:165, (18) SEQ ID NO:169, GAS, and SEQ ID NO:170, (19) SEQ ID NO:174, GAS, and SEQ ID NO:175, (20) SEQ ID NO:179, DAS, and SEQ ID NO:180, (21) SEQ ID NO:184, EAS, and SEQ ID NO:185, (22) SEQ ID NO:189, GAS, and SEQ ID NO:190, (23) SEQ ID NO:194, GAS, and SEQ ID NO:195, (24) SEQ ID NO:199, GAS, and SEQ ID NO:200, (25) SEQ ID NO:204, SNN, and SEQ ID NO:205, (26) SEQ ID NO:209, AAS, and SEQ ID NO:210, (27) SEQ ID NO:214, GPS, and SEQ ID NO:215, (28) SEQ ID NO:219, GPS, and SEQ ID NO:220, (29) SEQ ID NO:224, GAS, and SEQ ID NO:225, (30) SEQ ID NO:229, GAS, and SEQ ID NO:230, (31) SEQ ID NO:234, ENN, and SEQ ID NO:235, (32) SEQ ID NO:239, ENN, and SEQ ID NO:240, (33) SEQ ID NO:244, END, and SEQ ID NO:245, (34) SEQ ID NO:249, WAS, and SEQ ID NO:250, (35) SEQ ID NO:254, AVS, and SEQ ID NO:255, (36) SEQ ID NO:259, AAS, and SEQ ID NO:260, (37) SEQ ID NO:264, ENN, and SEQ ID NO:265, (38) SEQ ID NO:269, GAS, and SEQ ID NO:270, (39) SEQ ID NO:274, GAS, and SEQ ID NO:275, or (40) SEQ ID NO:279, GAS, and SEQ ID NO:280.

2. The pharmaceutical composition of claim 1, wherein the antibody or antigen-binding fragment comprises heavy chain CDRs (HCDR1-3) and light chain CDRs (LCDR1-3) sequences set forth respectively in (1) SEQ ID NOs:81-84, GAS, and SEQ ID NO:85; (2) SEQ ID NOs:86-89, GAS, and SEQ ID NO:90; (3) SEQ ID NOs:91-94, WAS, and SEQ ID NO:95; (4) SEQ ID NOs:96-99, GAS, and SEQ ID NO: 100; (5) SEQ ID NOs:101-104, GAS, and SEQ ID NO:105; (6) SEQ ID NOs:106-109, WAS, and SEQ ID NO:110; (7) SEQ ID NOs:111-114, SSY, and SEQ ID NO:115; (8) SEQ ID NOs:116-119, YAS, and SEQ ID NO:120; (9) SEQ ID NOs:121-124, GAS, and SEQ ID NO:125; (10) SEQ ID NOs:126-129, GAS, and SEQ ID NO:130; (11) SEQ ID NOs:131-134, GAS, and SEQ ID NO:135; (12) SEQ ID NOs:136-139, GAS, and SEQ ID NO:140; (13) SEQ ID NOs:141-144, DAS, and SEQ ID NO:145; (14) SEQ ID NOs:146-149, AVS, and SEQ ID NO:150; (15) SEQ ID NOs:151-154, GVS, and SEQ ID NO:155; (16) SEQ ID NOs:156-159, DNN, and SEQ ID NO:160; (17) SEQ ID NOs:161-164, ENN, and SEQ ID NO:165; (18) SEQ ID NOs:166-169, GAS, and SEQ ID NO:170; (19) SEQ ID NOs:171-174, GAS, and SEQ ID NO:175; (20) SEQ ID NOs:176-179, DAS, and SEQ ID NO:180; (21) SEQ ID NOs:181-184, EAS, and SEQ ID NO:185; (22) SEQ ID NOs:186-189, GAS, and SEQ ID NO:190; (23) SEQ ID NOs:191-194, GAS, and SEQ ID NO:195; (24) SEQ ID NOs:196-199, GAS, and SEQ ID NO:200; (25) SEQ ID NOs:201-204, SNN, and SEQ ID NO:205; (26) SEQ ID NOs:206-209, AAS, and SEQ ID NO:210; (27) SEQ ID NOs:211-214, GPS, and SEQ ID NO:215; (28) SEQ ID NOs:216-219, GPS, and SEQ ID NO:220; (29) SEQ ID NOs:221-224, GAS, and SEQ ID NO:225; (30) SEQ ID NOs:226-229, GAS, and SEQ ID NO:230; (31) SEQ ID NOs:231-234, ENN, and SEQ ID NO:235; (32) SEQ ID NOs:236-239, ENN, and SEQ ID NO:240; (33) SEQ ID NOs:241-244, END, and SEQ ID NO:245; (34) SEQ ID NOs:246-249, WAS, and SEQ ID NO:250; (35) SEQ ID NOs:251-254, AVS, and SEQ ID NO:255; (36) SEQ ID NOs:256-259, AAS, and SEQ ID NO:260; (37) SEQ ID NOs:261-264, ENN, and SEQ ID NO:265; (38) SEQ ID NOs:266-269, GAS, and SEQ ID NO:270; (39) SEQ ID NOs:271-274, GAS, and SEQ ID NO:275; or (40) SEQ ID NOs:276-279, GAS, and SEQ ID NO:280.

3. The pharmaceutical composition of claim 1-er-2, wherein the antibody or antigen-binding fragment comprises heavy chain variable region and light chain variable region sequences that are at least 95% identical, respectively, to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

4. The pharmaceutical composition of claim 1, wherein the antibody or antigen-binding fragment comprises heavy chain variable region and the light chain variable region sequences that are respectively identical to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

5. An antibody or antigen-binding fragment thereof that specifically binds to the S2 stem helix of a betacoronavirus, comprising heavy chain and light chain variable region sequences that, except for substitutions of one or more amino acid residues in the heavy chain framework region and/or the light chain framework region, are respectively identical to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

6. An antibody or antigen-binding fragment thereof that specifically binds to the S2 stem helix of a betacoronavirus, comprising (a) one or more non-natural amino acid residues in the Fc domain, and (b) heavy chain and light chain variable regions comprising: (i) heavy chain CDR sequences (HCDR1-3) as set forth respectively in (1) SEQ ID NOs:81-83, (2) SEQ ID NOs:86-88, (3) SEQ ID NOs:91-93, (4) SEQ ID NOs:96-98, (5) SEQ ID NOs:101-103, (6) SEQ ID NOs:106-108, (7) SEQ ID NOs:111-113, (8) SEQ ID NOs:116-118, (9) SEQ ID NOs:121-123, (10) SEQ ID NOs:126-128, (11) SEQ ID NOs:131-133, (12) SEQ ID NOs:136-138, (13) SEQ ID NOs:141-143, (14) SEQ ID NOs:146-148, (15) SEQ ID NOs:151-153, (16) SEQ ID NOs:156-158, (17) SEQ ID NOs:161-163, (18) SEQ ID NOs:166-168, (19) SEQ ID NOs:171-173, (20) SEQ ID NOs:176-178, (21) SEQ ID NOs:181-183, (22) SEQ ID NOs:186-188, (23) SEQ ID NOs:191-193, (24) SEQ ID NOs:196-198, (25) SEQ ID NOs:201-203, (26) SEQ ID NOs:206-208, (27) SEQ ID NOs:211-213, (28) SEQ ID NOs:216-218, (29) SEQ ID NOs:221-223, (30) SEQ ID NOs:226-228, (31) SEQ ID NOs:231-233, (32) SEQ ID NOs:236-238, (33) SEQ ID NOs:241-243, (34) SEQ ID NOs:246-248, (35) SEQ ID NOs:251-253, (36) SEQ ID NOs:256-258, (37) SEQ ID NOs:261-263, (38) SEQ ID NOs:266-268, (39) SEQ ID NOs:271-273, or SEQ ID NOs:276-278; and/or (ii) light chain CDR sequences (LCDR1-3) as set forth respectively in (1) SEQ ID NO:84, GAS, and SEQ ID NO:85, (2) SEQ ID NO:89, GAS, and SEQ ID NO:90, (3) SEQ ID NO:94, WAS, and SEQ ID NO:95, (4) SEQ ID NO:99, GAS, and SEQ ID NO:100, (5) SEQ ID NO:104, GAS, and SEQ ID NO:105, (6) SEQ ID NO:109, WAS, and SEQ ID NO:110, (7) SEQ ID NO: 114, SSY, and SEQ ID NO: 115, (8) SEQ ID NO: 119, YAS, and SEQ ID NO:120, (9) SEQ ID NO:124, GAS, and SEQ ID NO:125, (10) SEQ ID NO:129, GAS, and SEQ ID NO:130, (11) SEQ ID NO:134, GAS, and SEQ ID NO:135, (12) SEQ ID NO:139, GAS, and SEQ ID NO:140, (13) SEQ ID NO:144, DAS, and SEQ ID NO:145, (14) SEQ ID NO:149, AVS, and SEQ ID NO:150, (15) SEQ ID NO:154, GVS, and SEQ ID NO:155, (16) SEQ ID NO:159, DNN, and SEQ ID NO:160, (17) SEQ ID NO:164, ENN, and SEQ ID NO:165, (18) SEQ ID NO:169, GAS, and SEQ ID NO:170, (19) SEQ ID NO:174, GAS, and SEQ ID NO:175, (20) SEQ ID NO:179, DAS, and SEQ ID NO:180, (21) SEQ ID NO:184, EAS, and SEQ ID NO:185, (22) SEQ ID NO:189, GAS, and SEQ ID NO:190, (23) SEQ ID NO:194, GAS, and SEQ ID NO:195, (24) SEQ ID NO:199, GAS, and SEQ ID NO:200, (25) SEQ ID NO:204, SNN, and SEQ ID NO:205, (26) SEQ ID NO:209, AAS, and SEQ ID NO:210, (27) SEQ ID NO:214, GPS, and SEQ ID NO:215, (28) SEQ ID NO:219, GPS, and SEQ ID NO:220, (29) SEQ ID NO:224, GAS, and SEQ ID NO:225, (30) SEQ ID NO:229, GAS, and SEQ ID NO:230, (31) SEQ ID NO:234, ENN, and SEQ ID NO:235, (32) SEQ ID NO:239, ENN, and SEQ ID NO:240, (33) SEQ ID NO:244, END, and SEQ ID NO:245, (34) SEQ ID NO:249, WAS, and SEQ ID NO:250, (35) SEQ ID NO:254, AVS, and SEQ ID NO:255, (36) SEQ ID NO:259, AAS, and SEQ ID NO:260, (37) SEQ ID NO:264, ENN, and SEQ ID NO:265, (38) SEQ ID NO:269, GAS, and SEQ ID NO:270, (39) SEQ ID NO:274, GAS, and SEQ ID NO:275, or (40) SEQ ID NO:279, GAS, and SEQ ID NO:280.

7. A pharmaceutical composition or kit, comprising the antibody or antigen-binding fragment of claim 5.

8. A pharmaceutical composition or kit, comprising the antibody or antigen-binding fragment of claim 6.

9. The pharmaceutical composition of claim 1, wherein the second moiety is a polypeptide or a small organic molecule.

10. The pharmaceutical composition of claim 1, wherein the second moiety is a drug.

11. A polynucleotide, encoding the antibody or antigen-binding fragment thereof as recited in claim 1.

12. The polynucleotide of claim 11, encoding a heavy chain variable region and/or a light chain variable region sequences that are at least 95% identical, respectively, to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

13. The polynucleotide of claim 11, encoding a heavy chain variable region and/or a light chain variable region sequences that are respectively identical to (1) SEQ ID NOs:1 and 41, (2) SEQ ID NOs:2 and 42, (3) SEQ ID NOs:3 and 43, (4) SEQ ID NOs:4 and 44, (5) SEQ ID NOs:5 and 45, (6) SEQ ID NOs:6 and 46, (7) SEQ ID NOs:7 and 47, (8) SEQ ID NOs:8 and 48, (9) SEQ ID NOs:9 and 49, (10) SEQ ID NOs:10 and 50, (11) SEQ ID NOs:11 and 51, (12) SEQ ID NOs:12 and 52, (13) SEQ ID NOs:13 and 53, (14) SEQ ID NOs:14 and 54, (15) SEQ ID NOs:15 and 55, (16) SEQ ID NOs:16 and 56, (17) SEQ ID NOs:17 and 57, (18) SEQ ID NOs:18 and 58, (19) SEQ ID NOs:19 and 59, (20) SEQ ID NOs:20 and 60, (21) SEQ ID NOs:21 and 61, (22) SEQ ID NOs:22 and 62, (23) SEQ ID NOs:23 and 63, (24) SEQ ID NOs:24 and 64, (25) SEQ ID NOs:25 and 65, (26) SEQ ID NOs:26 and 66, (27) SEQ ID NOs:27 and 67, (28) SEQ ID NOs:28 and 68, (29) SEQ ID NOs:29 and 69, (30) SEQ ID NOs:30 and 70, (31) SEQ ID NOs:31 and 71, (32) SEQ ID NOs:32 and 72, (33) SEQ ID NOs:33 and 73, (34) SEQ ID NOs:34 and 74, (35) SEQ ID NOs:35 and 75, (36) SEQ ID NOs:36 and 76, (37) SEQ ID NOs:37 and 77, (38) SEQ ID NOs:38 and 78, (39) SEQ ID NOs:39 and 79, or (40) SEQ ID NOs:40 and 80.

14. The polynucleotide of claim 11, which is a cDNA.

15. A vector comprising the polynucleotide of claim 11.

16. A host cell harboring the vector of claim 15.

17. A method of producing an antibody or antigen-binding fragment thereof that specifically binds to the S2 stem helix of a betacoronavirus, comprising expressing the vector of claim 15 in a suitable host cell.

18. A method of treating or ameliorating symptoms associated with betacoronavirus infections in a subject, comprising administering to the subject afflicted with infection by one or more betacoronaviruses a pharmaceutical composition of claim 1.

19. The method of claim 18, wherein the betacoronavirus is SARS-CoV-2.

20. A method of diagnosing a betacoronavirus infection in a subject, comprising (a) obtaining a biological sample from the subject, and (b) contacting the sample with a S2 stem helix binding antibody or antigen-binding fragment as recited in claim 1 to detect a specific binding between an antigen in the sample and the antibody or antigen-binding fragment, thereby diagnosing a betacoronavirus infection in the subject.

21. The method of claim 20, wherein the biological sample is a blood sample or a saliva sample.
